(19) United States
(12) Patent Application Publication
(10) Pub. No.: US 2026/0157630 A1
(43) Pub. Date: Jun. 11, 2026

(54) SYSTEM AND METHOD FOR ANALYTE MONITORING

(71) Applicant: Senseonics, Incorporated, Germantown, MD (US)

(72) Inventors: Abhi Chavan, Germantown, MD (US); James Masciotti, Germantown, MD (US); Andrew DeHennis, Germantown, MD (US)

(73) Assignee: Senseonics, Incorporated, Germantown, MD (US)

(21) Appl. No.: 19/181,216

(22) Filed: Apr. 16, 2025

Related U.S. Application Data
(60) Provisional application No. 63/635,149, filed on Apr. 17, 2024.

(51) Int. Cl. A61B 5/00; A61B 5/145; A61B 5/1459

(52) U.S. Cl. CPC A61B 5/0022; A61B 5/14503; A61B 5/14532; A61B 5/1459

(57) ABSTRACT

Apparatuses, systems, and methods for autonomously-initiated analyte measurements and/or calculation of analyte concentrations based on the analyte measurements. An apparatus (e.g., an analyte sensor or a sensing device of the analyte sensor) may be configured to take and sets of sensor measurements at a first frequency having a period equal to a threshold number of cycles of the clock. The stored sets of sensor measurements may include first sets of sensor measurements at the first frequency and second sets of sensor measurements at a second frequency, and the first frequency is greater than the second frequency. Another apparatus (e.g., a transceiver, display device, and/or data management system) may be configured to receive sets of sensor measurements conveyed by the analyte sensor, calculate time stamps for the sets of sensor measurements, and calculate analyte concentrations based on the sets of sensor measurements and the calculated time stamps.

### CROSS-REFERENCE TO RELATED APPLICATION

The present application claims the benefit of priority to U.S. Provisional Application Ser. No. 63/635,149, filed on Apr. 17, 2024, which is incorporated herein by reference in its entirety.

### BACKGROUND

### Field of Invention

The present disclosure relates to an analyte monitoring system and method. More specifically, aspects of the present disclosure relate to an analyte monitoring system, which may include an analyte sensor that autonomously initiates and stores analyte measurements and a transceiver and/or display device that uses the analyte measurements to calculate analyte concentrations.

### Discussion of the Background

The prevalence of diabetes mellitus continues to increase in industrialized countries, and projections suggest that this figure will rise to 4.4% of the global population (366 million individuals) by the year 2030. Glycemic control is a key determinant of long-term outcomes in patients with diabetes, and poor glycemic control is associated with retinopathy, nephropathy and an increased risk of myocardial infarction, cerebrovascular accident, and peripheral vascular disease requiring limb amputation. Despite the development of new insulins and other classes of antidiabetic therapy, roughly half of all patients with diabetes do not achieve recommended target hemoglobin A1c (HbA1c) levels <7.0%.

Frequent self-monitoring of blood glucose (SMBG) is necessary to achieve tight glycemic control in patients with diabetes mellitus, particularly for those requiring insulin therapy. However, current blood (finger-stick) glucose tests are burdensome, and, even in structured clinical studies, patient adherence to the recommended frequency of SMBG decreases substantially over time. Moreover, finger-stick measurements only provide information about a single point in time and do not yield information regarding intraday fluctuations in blood glucose levels that may more closely correlate with some clinical outcomes.

Analyte monitoring systems (e.g., continuous glucose monitors (CGMs)) have been developed in an effort to overcome the limitations of finger-stick SMBG and thereby help improve patient outcomes. These systems enable increased frequency of glucose measurements and a better characterization of dynamic glucose fluctuations, including episodes of unrealized hypoglycemia. Furthermore, integration of CGMs with automated insulin pumps allows for establishment of a closed-loop “artificial pancreas” system to more closely approximate physiologic insulin delivery and to improve adherence.

Monitoring analyte measurements from a living body via wireless analyte monitoring sensor(s) may provide numerous health and research benefits. Improved analyte monitoring systems and methods are needed.

### SUMMARY

One aspect of the invention may provide an apparatus including a clock, one or more sensor elements, and a memory. The apparatus may be configured to cause the one or more sensor elements to take sets of sensor measurements at a first frequency. The first frequency may have a period equal to a threshold number of cycles of the clock. The apparatus may be configured to store the sets of sensor measurements in the memory. The stored sets of sensor measurements may include first sets of sensor measurements at the first frequency and second sets of sensor measurements at a second frequency. The first frequency may be greater than the second frequency.

In some aspects, the apparatus may further include a measurement scheduler and a measurement controller. The measurement scheduler may be configured to count the cycles of the clock and initiate measurement sequences at the first frequency. The measurement controller may be configured to, each time the measurement scheduler initiates a measurement sequence, cause the one or more sensor elements to take a set of sensor measurements and store the set of sensor measurements in the memory. In some aspects, the first sets of sensor measurements at the first frequency may be more recent sets of sensor measurements than the second sets of sensor measurements at the second frequency.

In some aspects, storing the sets of sensor measurements in the memory may include down-sampling previously-stored sets of sensor measurements. In some aspects, down-sampling previously-stored sets of sensor measurements may include discarding a previously-stored set of sensor measurements that is not an oldest set of sensor measurements. In some aspects, down-sampling previously-stored sets of sensor measurements may include discarding a previously-stored set of sensor measurements that is an oldest set of sensor measurements.

In some aspects, the apparatus may further include an indicator element including an analyte indicator having a detectable property that varies in accordance with at least a concentration of an analyte in proximity to the indicator element. In some aspects, the sets of sensor measurements may each include an analyte measurement based on the detectable property of the analyte indicator of the indicator element. In some aspects, the detectable property of the analyte indicator is a first detectable property, the first detectable property additionally varies in accordance with an effect on the analyte indicator, the indicator element further includes an interferent indicator having a second detectable property that varies in accordance with the effect on the analyte indicator, and the sets of sensor measurements each include an interferent measurement based on the second detectable property.

In some aspects, the one or more sensor elements may include a temperature transducer, and the sets of sensor measurements may each include a temperature measurement.

In some aspects, the apparatus may further include an interface device, the apparatus may be further configured to receive one or more measurement read requests using the interface device, and the apparatus may be further configured to, if the one or more measurement read requests are received, cause the interface device to convey the stored sets of sensor measurements. In some aspects, the apparatus is further configured to: receive a stop sensor measurement command using the interface device; if a stop sensor measurement command is received, stop causing the one or more sensor elements to take sets of sensor measurements at the first frequency; receive a start sensor measurement command using the interface device; and, if a start sensor measurement command is received, re-start causing the one or more sensor elements to take sets of sensor measurements at the first frequency. In some aspects, the interface device may be caused to convey the stored sets of sensor measurements while the analyte sensor is stopped from causing the one or more sensor elements to take sets of sensor measurements at the first frequency. In some aspects, the apparatus may be further configured to, if the one or more measurement read requests are received, cause the interface device to convey with the stored sets of sensor measurements a count of the cycles of the clock.

In some aspects, the apparatus may be further configured to store in the memory, for each of the stored sets of sensor measurements, a count of the cycles of the clock at the time the set of sensor measurements was taken.

Another aspect of the invention may provide a method including causing one or more sensor elements of an apparatus to take sets of sensor measurements at a first frequency. The first frequency may have a period equal to a threshold number of cycles of a clock of the apparatus. The method may include storing the sets of sensor measurements in a memory of the apparatus. The stored sets of sensor measurements may include first sets of sensor measurements at the first frequency and second sets of sensor measurements at a second frequency. The first frequency may be greater than the second frequency.

Still another aspect of the invention may provide an apparatus including an interface device. The apparatus may be configured to use the interface device to receive sets of sensor measurements conveyed by an analyte sensor. The analyte sensor may take sets of sensor measurements at a first frequency that has a period equal to a threshold number of cycles of a clock of the analyte sensor. The apparatus may be configured to calculate time stamps for the sets of sensor measurements. The apparatus may be configured to calculate analyte concentrations based on the sets of sensor measurements and the calculated time stamps.

In some aspects, each of the sets of sensor measurements may include timing information, and the apparatus may be configured to use at least the timing information of the sets of sensor measurements to calculate the time stamps for the sets of sensor measurements. In some aspects, the timing information of a set of sensor measurements may include a count of the cycles at the time the set of sensor measurements was taken. In some aspects, the timing information of a set of sensor measurements includes a number n for the set of sensor measurements. In some aspects, each of the sets of sensor measurements may include a temperature measurement, and the apparatus may be configured to use at least the timing information and one or more of the temperature measurements of the sets of sensor measurements to calculate the time stamps for the sets of sensor measurements. In some aspects, the apparatus may be configured to use at least the timing information and the one or more of the temperature measurements of the sets of sensor measurements and a characterization of a temperature dependence of the cycles of the clock of the analyte sensor to calculate the time stamps for the sets of sensor measurements.

In some aspects, the apparatus may be configured to use at least the timing information and the one or more of the temperature measurements of the sets of sensor measurements, the characterization of the temperature dependence of the cycles of the clock of the analyte sensor, and one or both of a time at which the apparatus conveyed a start sensor measurement command to the analyte sensor and a time at which the apparatus conveyed a stop measurement command to the analyte sensor to calculate the time stamps for the sets of sensor measurements. In some aspects, the apparatus may be configured to use at least the timing information and the one or more of the temperature measurements of the sets of sensor measurements, the characterization of the temperature dependence of the cycles of the clock of the analyte sensor, and the first frequency to calculate the time stamps for the sets of sensor measurements.

In some aspects, each of the sets of sensor measurements may include a voltage measurement, the voltage measurement may be a measurement of a voltage produced by a charge storage device of the analyte sensor, and the apparatus may be configured to use at least the timing information and one or more of the voltage measurements of the sets of sensor measurements to calculate the time stamps for the sets of sensor measurements. In some aspects, the apparatus may be configured to use at least the timing information and the one or more of the voltage measurements of the sets of sensor measurements and a characterization of a voltage dependence of the cycles of the clock of the analyte sensor to calculate the time stamps for the sets of sensor measurements. In some aspects, the apparatus may be configured to use at least the timing information and the one or more of the voltage measurements of the sets of sensor measurements, the characterization of the voltage dependence of the cycles of the clock of the analyte sensor, and one or both of a time at which the apparatus conveyed a start sensor measurement command to the analyte sensor and a time at which the apparatus conveyed a stop measurement command to the analyte sensor to calculate the time stamps for the sets of sensor measurements. In some aspects, the apparatus may be configured to use at least the timing information and the one or more of the voltage measurements of the sets of sensor measurements, the characterization of the voltage dependence of the cycles of the clock of the analyte sensor, and the first frequency to calculate the time stamps for the sets of sensor measurements.

In some aspects, the sets of sensor measurements include first sets of sensor measurements, which were stored by the analyte sensor at the first frequency, and second sets of sensor measurements, which were stored by the analyte sensor at a second frequency that is less than the first frequency. In some aspects, the apparatus may be configured to, in calculating the time stamps for the sets of sensor measurements, calculate time stamps for the first sets of sensor measurements and calculate time stamps for the second sets of sensor measurements. In some aspects, the first sets of sensor measurements may be more recent sets of sensor measurements than the second sets of sensor measurements.

In some aspects, the apparatus may be further configured to use the interface device to convey one or more measurement read requests, and the sets of sensor measurements may be received in response to the one or more measurement read requests. In some aspects, the apparatus may be further configured to: use the interface device to convey one or more stop sensor measurement commands before conveying the one or more measurement read requests; and use the interface device to convey one or more start sensor measurement commands after conveying the one or more measurement read requests and receiving the sets of sensor measurements.

In some aspects, the sets of sensor measurements may include measurements from a first sensing area and measurements from a second sensing area. In some aspects, the apparatus may be configured to, in calculating the analyte concentrations based on the sets of sensor measurements and the calculated time stamps, calculate individual analyte concentrations for the first sensing area, calculate individual analyte concentrations for the second sensing area, and calculate combined analyte concentrations based on at least the individual analyte concentrations for the first and second sensing areas.

In some aspects, the sets of sensor measurements may include sets of sensor measurements conveyed by a first sensing device of the analyte sensor and sets of sensor measurements conveyed by a second sensing device of the analyte sensor. In some aspects, the apparatus may be configured to calculate the analyte concentrations based on the sets of sensor measurements conveyed by the first sensing device of the analyte sensor, the sets of sensor measurements conveyed by the second sensing device of the analyte sensor, and the calculated time stamps.

In some aspects, the sets of sensor measurements conveyed by the first sensing device may include measurements from a first sensing area of the first sensing device and measurements from a second sensing area of the first sensing device. In some aspects, the sets of sensor measurements conveyed by the second sensing device may include measurements from a first sensing area of the second sensing device and measurements from a second sensing area of the second sensing device. In some aspects, the apparatus may be configured to, in calculating the analyte concentrations based on the sets of sensor measurements and the calculated time stamps: calculate individual analyte concentrations for the first sensing area of the first sensing device; calculate individual analyte concentrations for the second sensing area of the first sensing device; calculate individual analyte concentrations for the first sensing area of the second sensing device; calculate individual analyte concentrations for the second sensing area of the second sensing device; and calculate combined analyte concentrations based on at least the individual analyte concentrations for the first and second sensing areas of the first sensing device and the individual analyte concentrations for the first and second sensing areas of the second sensing device.

Yet another aspect of the invention may provide a method including using an interface device of an apparatus to receive sets of sensor measurements conveyed by an analyte sensor. The analyte sensor may take sets of sensor measurements at a first frequency that has a period equal to a threshold number of cycles of a clock of the analyte sensor. The method may include using the apparatus to calculate time stamps for the sets of sensor measurements. The method may include using the computer to calculate analyte concentrations based on the sets of sensor measurements and the calculated time stamps.

Further variations encompassed within the systems and methods are described in the detailed description of the invention below.

### BRIEF DESCRIPTION OF THE DRAWINGS

The accompanying drawings, which are incorporated herein and form part of the specification, illustrate various, non-limiting aspects of the present invention. In the drawings, like reference numbers indicate identical or functionally similar elements.

FIG. 1 is a schematic view illustrating an analyte monitoring system embodying aspects of the present invention.

FIG. 2 is a perspective view of an analyte sensor of the analyte monitoring system according to some aspects.

FIG. 3 is a side view of an analyte sensor of the analyte monitoring system according to some aspects.

FIG. 4 is a schematic view illustrating the layout of a semiconductor substrate of a sensing device of an analyte sensor of the analyte monitoring system embodying aspects of the present invention.

FIGS. 5A and 5B are schematic views each illustrating a sensing area of a sensing device of an analyte sensor embodying aspects of the present invention.

FIGS. 6A and 6B show the chemical structures of the analyte indicator and interference indicator, respectively, of indicator elements of an analyte sensor of the analyte monitoring system according to some aspects.

FIGS. 6C and 6D show fluorimeter readings demonstrating decrease in fluorescence intensity of analyte indicator molecule (excitation wavelength 380 nm) over time with simultaneous increase in the fluorescence intensity of interferent indicator molecule (excitation wavelength 470 nm) over time.

FIGS. 6E and 6F illustrate the white color of an indicator element with no oxidation and the yellow color of the oxidized indicator element, respectively, for an indicator element including an interferent indicator embodying aspects of the present invention.

FIG. 6G illustrates a decrease in the intensity or amount of light emitted by an analyte indicator over time according to aspects of the present invention.

FIG. 6H illustrates an increase in the absorption of an indicator element over time and a decrease in the intensity or amount of the second excitation light reflected by the indicator element over time according to aspects of the present invention.

FIG. 7A is a perspective view of an analyte sensor including a charge storage device embodying aspects of the present invention.

FIGS. 7B and 7C are side views of an analyte sensor including a charge storage device embodying aspects of the present invention.

FIG. 8A is a block diagram illustrating the main functional blocks of the circuitry of a sensing device of an analyte sensor embodying aspects of the present invention.

FIGS. 8B-1, 8B-2, and 8B-3 show sections of a block diagram illustrating functional blocks of the circuitry of a sensing device of an analyte sensor embodying aspects of the present invention.

FIG. 9 is a schematic view illustrating the layout of a semiconductor substrate of a sensing device of an analyte sensor of the analyte monitoring system embodying aspects of the present invention.

FIG. 10 is cross-sectional, perspective view of a transceiver embodying aspects of the invention.

FIG. 11 is an exploded, perspective view of a transceiver embodying aspects of the invention.

FIG. 12 is a schematic view illustrating a transceiver embodying aspects of the present invention.

FIG. 13 illustrates a block diagram of a display device of the analyte monitoring system embodying aspects of the present invention.

FIG. 14 illustrates a block diagram of a computer (e.g., of the display device) of the analyte monitoring system according to some aspects.

FIG. 15 illustrates an example of a home screen illustrative display of a medical mobile application embodying aspects of various aspects of the present invention.

FIG. 16 shows an example of use of the analyte monitoring system as a continuous analyte monitoring system according to some aspects in which the analyte sensor is powered by the transceiver.

FIG. 17 shows an example of use of the analyte monitoring system as a continuous analyte monitoring system according to some aspects in which the analyte sensor is powered by a battery of the analyte sensor.

FIG. 18 shows an example of use of the analyte monitoring system as a flash analyte monitoring system according to some aspects in which the display device calculates analyte concentrations.

FIG. 19 shows an example of use of the analyte monitoring system as a flash analyte monitoring system according to some aspects in which the transceiver calculates analyte concentrations.

FIG. 20 shows an example of use of the analyte monitoring system as a flash analyte monitoring system according to some aspects in which the data management system (DMS) calculates analyte concentrations.

FIGS. 21A and 21B show example configurations of a memory of a sensing device of an analyte sensor according to some aspects.

FIGS. 21C and 21D show an example of sets of sensor measurements stored in a memory of an analyte sensor capable of storing up to 40 sets of sensor measurements at various instances of time according to some aspects.

FIG. 22A is a flowchart illustrating a process according to some aspects.

FIG. 22B is a flowchart illustrating a process according to some aspects.

FIG. 23 is a flowchart illustrating a process according to some aspects.

FIG. 24 is a flowchart illustrating a process according to some aspects.

### DETAILED DESCRIPTION OF PREFERRED EMBODIMENTS

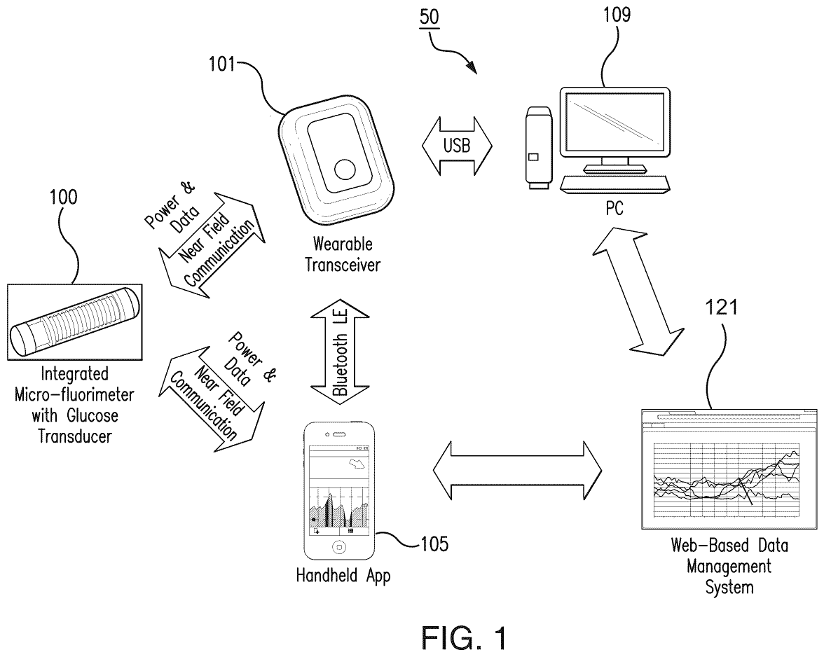

FIG. 1 is a schematic view of an exemplary analyte monitoring system 50 embodying aspects of the present invention. In some aspects, the analyte monitoring system 50 may be a continuous analyte monitoring system (e.g., a continuous glucose monitoring system). In some aspects, the analyte monitoring system 50 may include an analyte sensor 100, a transceiver 101, a display device 105, a personal computer 109, and/or a data management system (DMS) 121 hosted by a remote server or network attached storage hardware.

In some aspects, the sensor 100 may be small, fully subcutaneously implantable sensor measures analyte (e.g., glucose) concentrations in a medium (e.g., interstitial fluid) of a living animal (e.g., a living human). However, this is not required, and, in some alternative aspects, the sensor 100 may be a partially implantable (e.g., transcutaneous) sensor or a fully external sensor. In some aspects, the analyte sensor 100 may be powered by (a) one or more charge storage devices (e.g., one or more batteries) included in the analyte sensor 100 and/or (b) power received from a source (e.g., the transceiver 101 and/or the display device 105) external to the analyte sensor 100. In some non-limiting aspects, the analyte sensor 100 may include one or more optical sensors (e.g., one or more fluorometers). In some aspects, the analyte sensor 100 may be chemical or biochemical sensors. In some aspects, the analyte sensor 100 may be a radio frequency identification (RFID) device.

In some aspects, the transceiver 101 may be an externally worn transceiver (e.g., attached via an armband, wristband, waistband, or adhesive patch). In some aspects, the transceiver 101 may remotely power and/or communicate with the sensor to initiate and receive the measurements (e.g., via near field communication (NFC) or far field communication). However, this is not required, and, in some alternative aspects, the transceiver 101 may power and/or communicate with the sensor 100 via one or more wired connections. In some aspects, the transceiver 101 may be a smartphone (e.g., an NFC-enabled smartphone). In some aspects, the transceiver 101 may communicate information (e.g., one or more analyte concentrations) wirelessly (e.g., via a Bluetooth™ communication standard such as, for example and without limitation Bluetooth Low Energy) to a mobile medical application running on a display device 105 (e.g., a smartphone such as, for example, an NFC-enabled smartphone). In some aspects, the analyte monitoring system 50 may include a web interface for plotting and sharing of uploaded data.

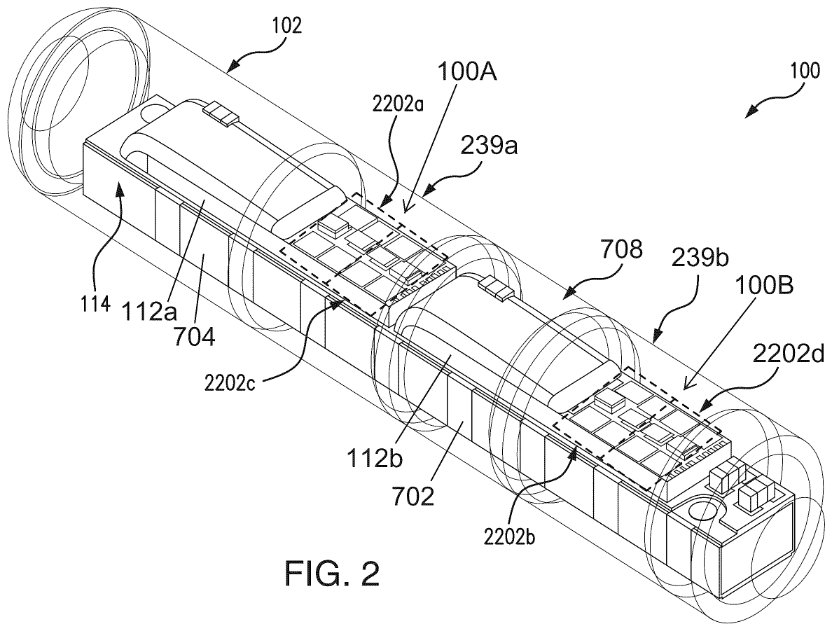

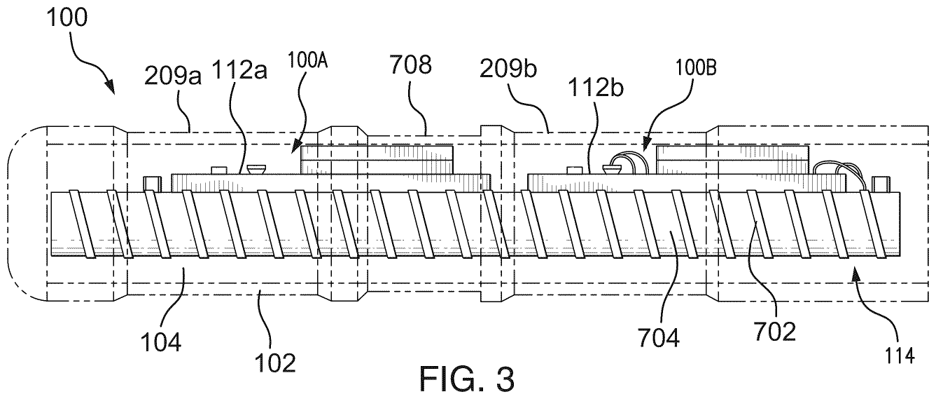

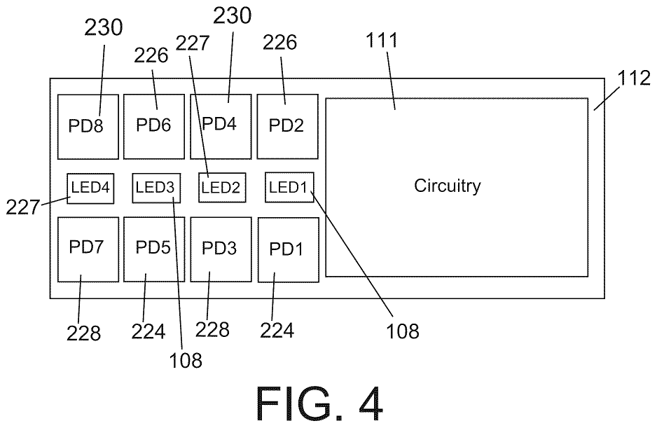

FIGS. 2 and 3 are perspective and side views, respectively, of analyte sensors 100 in accordance with aspects of the invention. In some aspects, as shown in FIGS. 2 and 3, the analyte sensor 100 may include a sensor housing 102 (i.e., body, shell, capsule, or encasement), which may be rigid and biocompatible. In some aspects, the sensor housing 102 may be a silicon tube. However, this is not required, and, in other aspects, different materials and/or shapes may be used for the sensor housing 102. In some aspects, as shown in FIG. 3, the analyte sensor 100 may include a transmissive optical cavity 104 (e.g., within the sensor housing 102). In some aspects, the transmissive optical cavity 104 may be formed from a suitable, optically transmissive polymer material, such as, for example, acrylic polymers (e.g., polymethylmethacrylate (PMMA)). However, this is not required, and, in other aspects, different materials may be used for the transmissive optical cavity 104.

In some aspects, as shown in FIGS. 2 and 3, the analyte sensor 100 may include one or more sensing devices. For example, as illustrated in FIGS. 2 and 3, the analyte sensor 100 may include first and second sensing devices 100A and 100B. However, in some alternative aspects, the analyte sensor 100 may include a different number of sensing devices (e.g., one, three, four, five, ten, etc.).

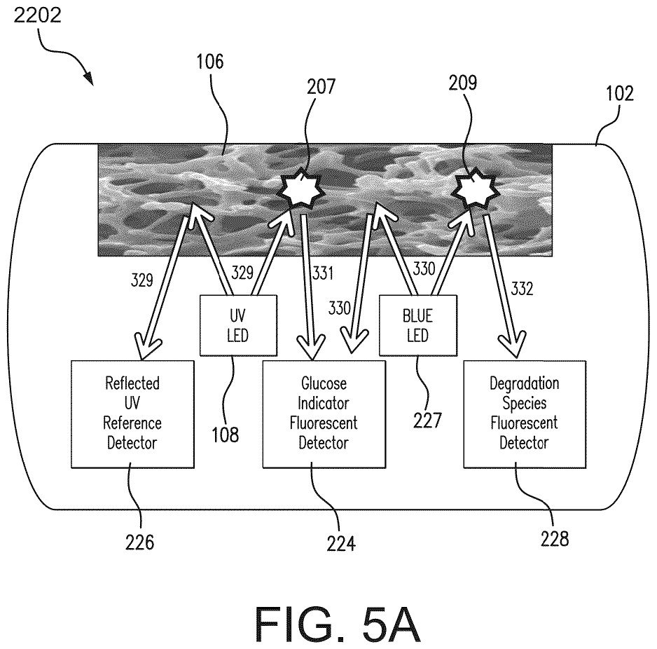

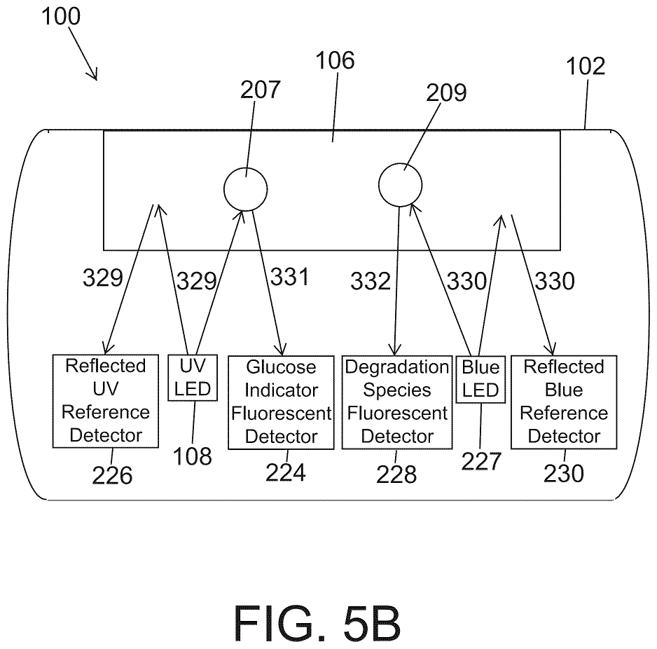

In some aspects, the analyte sensor 100 may include one or more indicator elements 106, which may be, for example, polymer grafts or hydrogels coated, diffused, adhered, embedded, or grown on or in one or more portions of the exterior surface of the sensor housing 102. In some aspects, as shown in FIGS. 2 and 3, the sensor housing 102 may include one or more cutouts or recesses 239a and 239b. In some aspects, one or more indicator elements 106 (described below with reference to FIGS. 5A and 5B) may be located (partially or entirely) in the cutouts or recesses 239a and 239b. In some aspects, the first sensing device 100A may include a first indicator element 106a, and the second sensing device 100B may include a second indicator element 106b. In some aspects, the indicator elements 106a and 106b may be, for example and without limitation, hydrogels on the sensor housing 102. In some aspects, the one or more indicator elements 106 may be porous and may allow the analyte (e.g., glucose) in a medium (e.g., interstitial fluid) to diffuse into the one or more indicator elements 106.

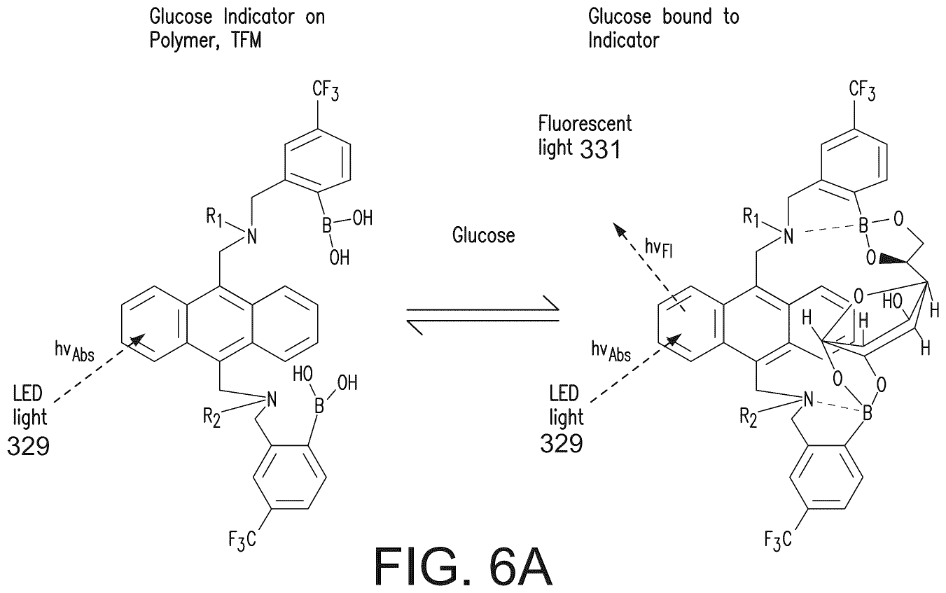

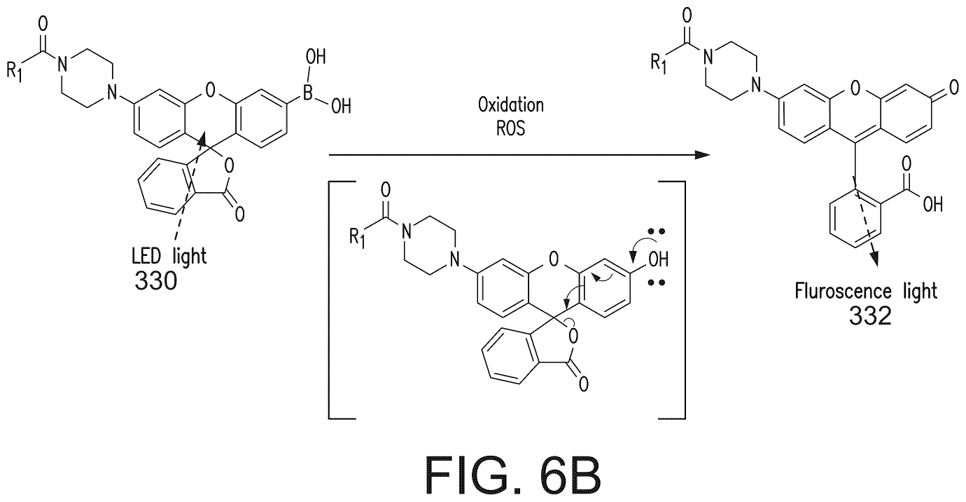

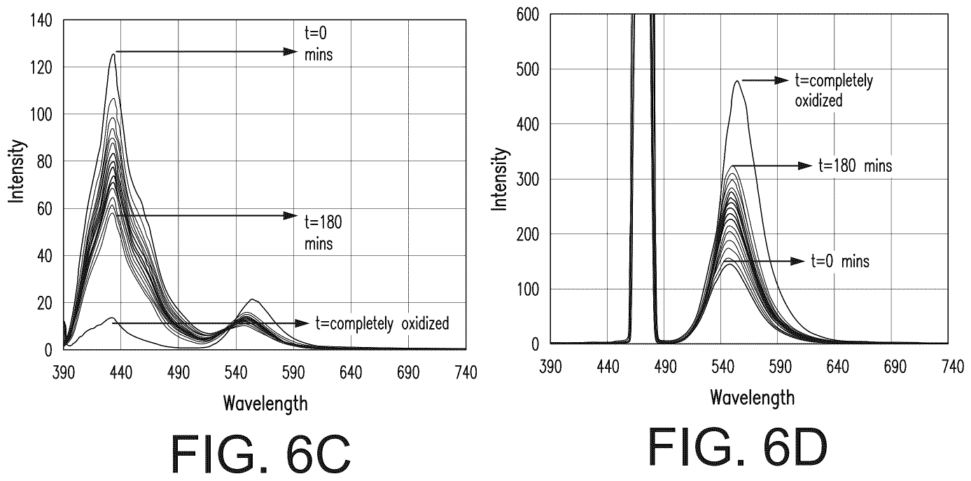

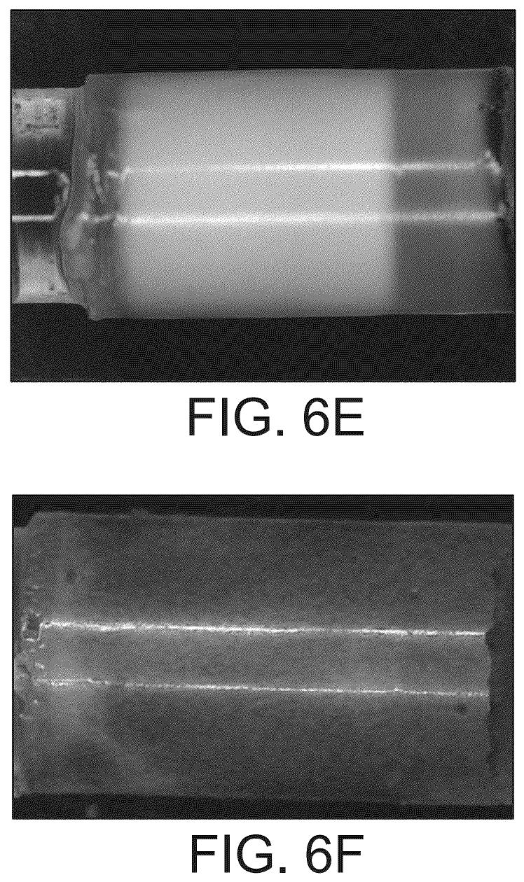

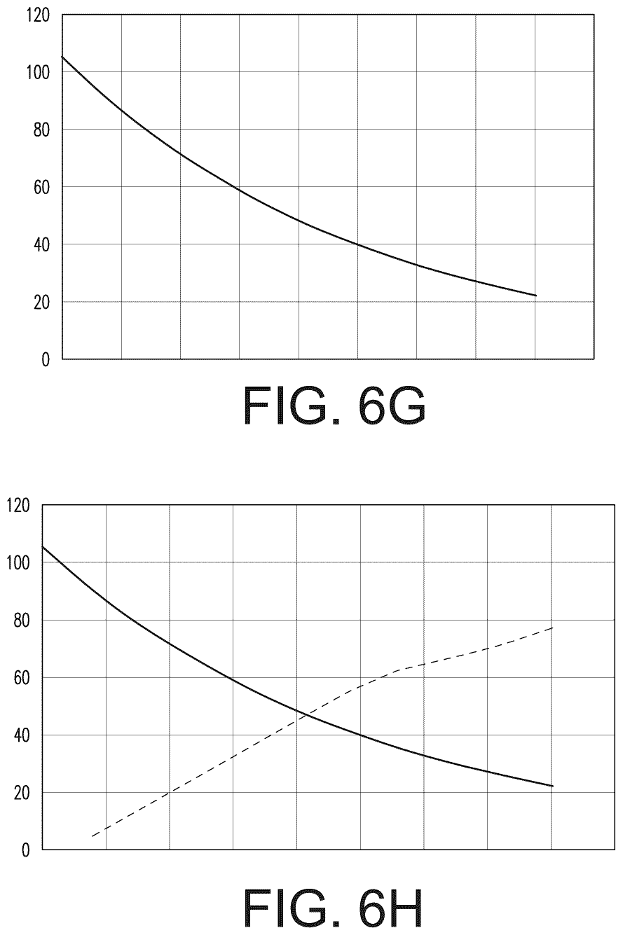

In some aspects, as shown in FIGS. 5A and 5B, the indicator elements 106 (e.g., indicator elements 106a and 106b) of the sensing devices 100A and 100B may each include an analyte indicator 207 and an interferent indicator 209 (e.g., a degradation indicator). In some aspects, the sensing devices 100A and 100B of the analyte sensor 100 may use the analyte indicator 207 to measure the presence, amount, and/or concentration of an analyte (e.g., glucose, oxygen, cardiac markers, low-density lipoprotein (LDL), high-density lipoprotein (HDL), or triglycerides). In some aspects, the analyte indicator 207 of the indicator elements 106a and 106b may have the chemical structure shown in FIG. 6A. In some aspects, the sensing devices 100A and 100B of the analyte sensor 100 may use the interferent indicator 209 to measure in vivo (e.g., ROS induced) signal degradation, which may enable reduction of the frequency at which calibration based on reference analyte measurements (e.g., finger stick blood glucose measurements) is carried out. In some aspects, the interferent indicator 209 of the indicator elements 106a and 106b may have the chemical structure shown in FIG. 6B. In some aspects, in the indicator elements 106a and 106b, the analyte indicator 207 and the interferent indicator 209 may be copolymerized into a single biocompatible hydrogel. In some aspects, the analyte indicator 207 and the interferent indicator 209 may have negligible spectral overlap and undergo similar degradation (e.g., similar degradation of boronic acids) in vivo.

In some aspects, the analyte indicator 207 may have one or more detectable properties (e.g., optical properties) that vary in accordance with (i) the amount or concentration of the analyte in proximity to the indicator element 106 and (ii) an effect on the analyte indicator 207 (e.g., changes to the analyte indicator 207). In some aspects, the changes to the analyte indicator 207 may comprise the extent to which the analyte indicator 207 has degraded. In some aspects, the degradation may be (at least in part) ROS-induced oxidation. In some aspects, the analyte indicator 207 may include one or more analyte indicator molecules (e.g., fluorescent analyte indicator molecules), which may be distributed throughout the indicator element 106. In some aspects, the analyte indicator 207 may be a phenylboronic-based analyte indicator. However, a phenylboronic-based analyte indicator is not required, and, in some alternative aspects, the analyte sensor 100 may include a different analyte indicator, such as, for example and without limitation, glucose oxidase-based indicators, glucose dehydrogenase-based indicators, and glucose binding protein-based indicators.

In some aspects, the interferent indicator 209 may have one or more detectable properties (e.g., optical properties) that vary in accordance with changes to the interferent indicator 209. In some aspects, the interferent indicator 209 is not sensitive to the amount of concentration of the analyte in proximity to the indicator element 106. That is, in some aspects, the one or more detectable properties of the interferent indicator 209 do not vary in accordance with the amount or concentration of the analyte in proximity to the indicator element 106. However, this is not required, and, in some alternative aspects, the one or more detectable properties of the interferent indicator 209 may vary in accordance with the amount or concentration of the analyte in proximity to the indicator element 106.

In some aspects, the changes to the interferent indicator 209 may comprise the extent to which the interferent indicator 209 has degraded. In some aspects, the degradation may be (at least in part) ROS-induced oxidation. In some aspects, the interferent indicator 209 may include one or more interferent indicator molecules (e.g., fluorescent interferent indicator molecules), which may be distributed throughout the indicator element 106. In some aspects, the interferent indicator 209 may be a phenylboronic-based interferent indicator. However, a phenylboronic-based interferent indicator is not required, and, in some alternative aspects, the analyte sensor 100 may include a different interferent indicator, such as, for example and without limitation, amplex red-based interferent indicators, dichlorodihydrofluorescein-based indicators, dihydrorhodamine-based indicators, and scopoletin-based interferent indicators.

In some aspects, the analyte sensor 100 may measure changes to the analyte indicator 207 of an indicator element 106 indirectly using the interferent indicator 209 of the indicator element 106, which may by sensitive to degradation by reactive oxygen species (ROS) but not sensitive to the analyte. In some aspects, the interferent indicator 209 may have one or more optical properties that change with extent of oxidation and may be used as a reference dye for measuring and correcting for extent of oxidation of the analyte indicator. In some aspects, the extent to which the interferent indicator 209 has degraded may correspond to the extent to which the analyte indicator 207 has degraded. For example, in aspects, the extent to which the interferent indicator 209 has degraded may be proportional to the extent to which the analyte indicator 207 has degraded. In some aspects, the extent to which the analyte indicator 207 has degraded may be calculated based on the extent to which the interferent indicator 209 has degraded. In some aspects, the analyte monitoring system 50 may correct for changes in the analyte indicator 207 using an empiric correlation established through laboratory testing.

In some aspects, as shown in FIG. 2, the analyte sensor 100 may include multiple sensing areas 2202 (e.g., sensing areas 2202a, 2202b, 2202c, and 2202d). In some aspects, as shown in FIG. 2, the sensing areas 2202a and 2202c may be long end distal (LED) and long end central (LEC) sensing areas of the analyte sensor 100, respectively, and the sensing areas 2202b and 2202d may be short end central (SEC) and short end distal (SED) sensing areas of the analyte sensor, respectively. In some aspects, as shown in FIG. 2, the first sensing device 100A may include sensing areas 2202a and 2202c, and the second sensing device 100B may include sensing areas 2202b and 2202d. In some aspects, the sensing areas 2202 may each include a measurement electronics (e.g., optical measurement electronics). In some aspects, the measurement electronics in each of the sensing areas 2202 may include one or more light sources and/or one or more photodetectors. For example, in some aspects, as shown in FIG. 5A, the sensing areas 2202 of the analyte sensor 100 may include one or more first light sources 108 that emit first excitation light 329 over a wavelength range that interacts with the analyte indicator 207 in the indicator element 106. In some aspects, the first excitation light 329 may be ultraviolet (UV) light. In some aspects, the analyte sensor 100 may include one or more second light sources 227 that emit second excitation light 330 over a wavelength range that interacts with the interferent indicator 209 in the indicator element 106. In some non-limiting aspects, the second excitation light 330 may be blue light. In some aspects, the sensing devices 100A and 100B may each include one or more temperature transducers.

In some aspects, as shown in FIG. 6A, an analyte (e.g., glucose) may bind reversibly to the analyte indicator 207, the analyte indicator 207 to which the analyte is bound may emit first emission light 331 (e.g., fluorescent light) when irradiated by the first excitation light 329, and the analyte indicator 207 to which the analyte is not bound may not emit light (or emit only a small amount of light) when irradiated by the first excitation light 329. In some aspects, as shown in FIG. 6B, oxidation of the interferent indicator 209 may cause the interferent indicator 209 to emit second emission light 332 (e.g., when irradiated by the second excitation light 330). In some aspects, oxidation of the interferent indicator 209 may additionally or alternatively cause the absorption of the interferent indicator 209 (e.g., absorption of the second excitation light 330 by the interferent indicator 209) to change. In some aspects, as shown in FIG. 2, one or more sensing areas 2202 (e.g., sensing areas 2202a and 2202c) may interact with (e.g., emit first and second excitation lights 329 and 330 to and measure first and second emission lights 331 and 332 emitted by) a first indicator element 106a, and one or more different sensing areas 2202 (e.g., sensing areas 2202b and 2202d) may interact with a second indicator element 106b.

In some aspects, as shown in FIG. 5A, the sensing areas 2202 of the analyte sensor 100 may also include one or more photodetectors 224, 226, 228 (e.g., photodiodes, phototransistors, photoresistors, or other photosensitive elements). In some aspects, the sensing areas 2202 of the analyte sensor 100 may include one or more signal photodetectors 224 sensitive to first emission light 331 (e.g., fluorescent light) emitted by the analyte indicator 207 of the indicator element 106 such that a signal generated by a signal photodetector 224 is indicative of the level of first emission light 331 of the analyte indicator 207 and, thus, the amount of analyte of interest (e.g., glucose). In some aspects, the sensing areas 2202 of the analyte sensor 100 may include one or more reference photodetectors 226 sensitive to first excitation light 329 that may be reflected from the indicator element 106 such that a signal generated by a photodetector 226 in response thereto is indicative of the level of reflected first excitation light 329. In some aspects, the analyte sensor 100 may include one or more interferent photodetectors 228 sensitive to second emission light 332 (e.g., fluorescent light) emitted by the interferent indicator 209 of the indicator element 106 such that a signal generated by an interferent photodetector 228 in response thereto that is indicative of the level of second emission light 332 of the interferent indicator 209 and, thus, the amount of degradation (e.g., oxidation). In some aspects, the one or more signal photodetectors 224 may be sensitive to second excitation light 330 that may be reflected from the indicator element 106. In this way, the one or more signal photodetectors 224 may act as reference photodetectors when the one or more light sources 227 are emitting second excitation light 330.

However, it is not required that the one or more signal photodetectors 224 act as reference photodetectors when the one or more light sources 227 are emitting second excitation light 330. In some alternative aspects, as shown in FIG. 5B, the sensing areas 2202 of the analyte sensor 100 may include one or more second reference photodetectors 230 that act as reference photodetectors when the one or more light sources 227 are emitting second excitation light 330. In some aspects, the one or more second reference photodetectors 230 may be sensitive to second excitation light 330 that may be reflected from the indicator element 106 such that a signal generated by a photodetector 230 in response thereto is indicative of the level of reflected second excitation light 330.

In some aspects, the first excitation light 329 may be over a first wavelength range, and the second excitation light 330 over a second wavelength range, which may different than the first wavelength range. In some aspects, the first and second wavelength ranges do not overlap, but this not required, and, in some alternative aspects, the first and second wavelength ranges may overlap. In some aspects, the first emission light 331 may be over a third wavelength range, and the second emission light 332 may be over a fourth wavelength range, which may be different than the third wavelength range. In some aspects, the third and fourth wavelength ranges do not overlap, but this is not required, and, in some alternative aspects, the third and fourth wavelength ranges may overlap. In some aspects, the first and third wavelength ranges may be different. In some aspects, the first and third wavelength ranges do not overlap, but this is not required, and, in some alternative aspects, the first and third wavelength ranges may overlap. In some aspects, the second and fourth wavelength ranges may be different. In some aspects, the second and fourth wavelength ranges do not overlap, but this is not required, and, in some alternative aspects, the second and fourth wavelength ranges may overlap. In some aspects, the second and third wavelength ranges may be different. In some aspects, the second and third wavelength ranges may overlap, but this is not required and, in some alternative aspects, the second and third wavelength ranges do not overlap. In some further alternative aspects, the second and third wavelength ranges may be the same.

In some aspects, one or more of the photodetectors 224, 226, 228, 230 may be covered by one or more filters that allow only a certain subset of wavelengths of light to pass through and reflect (or absorb) the remaining wavelengths. In some aspects, one or more filters on the one or more signal photodetectors 224 may allow only a subset of wavelengths corresponding to first emission light 331 and/or the reflected second excitation light 330. In some aspects, one or more filters on the one or more reference photodetectors 226 may allow only a subset of wavelengths corresponding to the reflected first excitation light 329. In some aspects, one or more filters on the one or more interferent photodetectors 228 may allow only a subset of wavelengths corresponding to second emission light 332. In some aspects in which the analyte sensor 100 includes one or more second reference photodetectors 230, one or more filters on the one or more second reference photodetectors 230 may allow only a subset of wavelengths corresponding to the reflected second excitation light 330.

In some aspects, the intensity or amount of emission light (e.g., first emission light 331) emitted by the analyte indicator 207 may change (e.g., increase or decrease) as degradation of the analyte indicator 207 increases. In some aspects, as shown in FIG. 6C, the intensity or amount of emission light (e.g., first emission light 331) emitted by an analyte indicator 207 including the analyte indicator molecule shown in FIG. 6A may decrease as degradation of the analyte indicator 207 increases over time.

In some aspects, the intensity or amount of emission light (e.g., second emission light 332) emitted by the interferent indicator 209 may change (e.g., increase or decrease) as degradation of the interferent indicator 209 increases. In some aspects, the extent of the degradation of the interferent indicator 209 may correspond to the extent of degradation of the analyte indicator 207. Accordingly, in some aspects, the extent of the change in the intensity or amount of emission light emitted by the interferent indicator 209 may correspond to the change in the intensity or amount of emission light emitted by the analyte indicator 207. In some aspects, as shown in FIG. 6D, the intensity or amount of emission light (e.g., second emission light 332) emitted by an interferent indicator 209 including the interferent indicator molecule shown in FIG. 6B may increase as degradation of the interferent indicator 209 increases over time. However, this is not required, and, in some alternative aspects, the intensity or amount of emission light (e.g., second emission light 332) emitted by an interferent indicator 209 may decrease as degradation of the interferent indicator 209 increases over time.

In some aspects, in addition to (or as an alternative to) the intensity or amount of emission light (e.g., second emission light 332) emitted by the interferent indicator 209 changing as degradation of the interferent indicator 209 increases, the absorption of the interferent indicator 209 may change (e.g., increase or decrease) as degradation of the interferent indicator 209 increases. In some aspects, the extent of the degradation of the interferent indicator 209 may correspond to the extent of degradation of the analyte indicator 207. Accordingly, in some aspects, the extent of the change in the absorption of the interferent indicator 209 (e.g., as measured by the amount of second excitation light 330 reflected from and not absorbed by the indicator element 106) may correspond to the change in the intensity or amount of emission light emitted by the analyte indicator 207. In some aspects, as degradation (e.g., oxidation) of the interferent indicator 209 increases, the color of the interferent indicator 209 (and, therefore, the color of the indicator element 106 including the interferent indicator 209) may change. For example, in some aspects, the color of the indicator element 106 may change from white with no oxidation, as shown in FIG. 6E, to yellow when oxidized, as shown in FIG. 6F. However, a change from white to yellow is not required, and, in some alternative aspects, different color changes may occur with degradation (e.g., white to yellow, white to orange, yellow to red, orange to brown, etc.). In some aspects, the change in the color of the interferent indicator 209 (and, therefore, the color of the indicator element 106 including the interferent indicator 209) may change the absorption of the interferent indicator 209 (and, therefore, the absorption of the indicator element 106 including the interferent indicator 209).

In some aspects, as shown by FIG. 6G, the intensity or amount of the emission light 331 emitted by the analyte indicator 207 may decrease over time (e.g., as degradation, such as oxidation, of the analyte indicator 207 increases). In some aspects, as shown by the dashed line of FIG. 6H, the absorption of the indicator element 106 may increase over time (e.g., as degradation, such as oxidation, of the interferent indicator 209 increases). In some aspects, as shown by the solid line of FIG. 6H, the intensity or amount of the second excitation light 330 reflected by the indicator element 106 may decrease over time (e.g., as degradation, such as oxidation, of the interferent indicator 209 increases). In some aspects, as shown in FIGS. 6G and 6H, the increase in the absorption of the indicator element 106 and the decrease in the intensity or amount of the second excitation light 330 reflected by the indicator element 106 may correspond to the decrease in the intensity or amount of emission light 331 emitted by the analyte indicator 207.

In some aspects, as shown in FIGS. 2 and 3, the analyte sensor 100 may include one or more substrates 112 (e.g., a first substrate 112a and a second substrate 112b). In some aspects, the first sensing device 100A may include the first substrate 112a, and the second sensing device 100B may include the second substrate 112b. In some aspects, as shown in FIG. 4, the substrates 112 (e.g., the first substrate 112a and the second substrate 112b) may be circuit boards (e.g., a printed circuit boards (PCBs) or flexible PCBs) on which one or more of the circuit components 111 (e.g., analog and/or digital circuit components) may be mounted or otherwise attached. However, in some alternative aspects, the substrates 112 may be semiconductor substrates having one or more of the circuit components 111 fabricated therein. For instance, the fabricated circuit components may include analog and/or digital circuitry. Also, in some aspects in which the substrate 112 is a semiconductor substrate, in addition to the circuit components fabricated in the semiconductor substrate, circuit components may be mounted or otherwise attached to the semiconductor substrate. In other words, in some semiconductor substrate aspects, a portion or all of the circuit components 111, which may include discrete circuit elements, an integrated circuit (e.g., an application specific integrated circuit (ASIC)) and/or other electronic components (e.g., a non-volatile memory), may be fabricated in the semiconductor substrate with the remainder of the circuit components 111 secured to the semiconductor substrate, which may provide communication paths between the various secured components.

In some aspects, as shown in FIG. 2, the substrates 112 may each include the measurement electronics of two or more sensing areas 2202 (e.g., the measurement electronics of the sensing area 2202a and 2202c may be mounted on and/or fabricated in the first substrate 112a, and the measurement electronics of the sensing area 2202b and 2202d may be mounted on and/or fabricated in the second substrate 112b). In some aspects, as shown in FIG. 4, the substrates 112 may include (i) a first set of one or more first light sources 108, one or more second light sources 227, one or more signal photodetectors 224, one or more reference photodetectors 226, one or more interferent photodetectors 228, and/or one or more second reference photodetectors 230 mount on and/or fabricated in the substrate 112 for one sensing area 2202 and (ii) a second set of one or more first light sources 108, one or more second light sources 227, one or more signal photodetectors 224, one or more reference photodetectors 226, one or more interferent photodetectors 228, and/or one or more second reference photodetectors 230 mount on and/or fabricated in the substrate 112 for another sensing area 2202. In one aspect, the first and second light sources 108 and 227 may be mounted on the substrate 112, the photodetectors 224, 226, 228, and/or 230 may be fabricated in the substrate 112, and all or a portion of the circuit components 111 may be fabricated within the substrate 112.

In some aspects, the indicator elements 106, the first and second light sources 108 and 227, the photodetectors 224, 226, 228, and/or 230, the circuit components 111, and the substrates 112 of the sensing devices 100A and 100B of the analyte sensor 100 may include some or all of the features described in one or more of U.S. application Ser. No. 13/761,839, filed on Feb. 7, 2013, U.S. application Ser. No. 13/937,871, filed on Jul. 9, 2013, U.S. application Ser. No. 13/650,016, filed on Oct. 11, 2012, and U.S. application Ser. No. 14/142,017, filed on Dec. 27, 2013, all of which are incorporated by reference in their entireties. Similarly, the structure, function, and/or features of the sensor housing 102, analyte sensor 100, and/or transceiver 101 may be as described in one or more of U.S. application Ser. Nos. 13/761,839, 13/937,871, 13/650,016, and 14/142,017. For instance, the sensor housing 102 may have one or more hydrophobic, hydrophilic, opaque, and/or immune response blocking membranes or layers on the exterior thereof.

In some aspects, the analyte sensor 100 may sense an analyte (e.g., glucose) in each of the multiple sensing areas 2202 (e.g., each of the sensing areas 2202a-2202d). In some aspects, the multiple sensing areas 2202 may be redundant sensing areas. In some aspects, in each of the sensing areas 2202, the analyte indicator 207 may be excited by first excitation light 329 emitted by a first light source 108 (e.g., a UV LED), and the interferent indicator 209 may be excited by second excitation light 330 emitted by a light source 227 (e.g., a blue LED). In some aspects, the first excitation light 329 and the first emission light 331 emitted by the analyte indicator 207 may be measured by one or more first reference photodetectors 226 (e.g., one or more UV filter coated photodiodes) and one or more signal photodetectors 224 (e.g., one or more blue filter coated photodiodes) respectively. In some aspects, the second excitation light 330 may be measured by one or more signal photodetectors 224 (see FIG. 5A) or one or more second reference photodetectors 230 (see FIG. 5B), which may be, for example, one or more blue filter coated photodiodes. In some aspects, the second emission light 332 emitted by the interferent indicator 209 may be measured by one or more interferent photodetector 228 (e.g., one or more yellow filter coated photodiodes).

In some aspects, the analyte sensor 100 shown in FIG. 2 may combine an interferent indicator 209 used to measure oxidation and redundant sensing areas 2202a-2202d to obtain analyte values using weighted averaging. In some aspects, the analyte monitoring system 50 (e.g., the transceiver 101 and/or the display device 105 of the analyte monitoring system 50) may integrate the oxidation measurements and the analyte measurements into an analyte calculation model that allows for reduced calibration frequency (e.g., one calibration per week after day 14). In some aspects, the analyte monitoring system 50 may selectively utilize information (e.g., measurements) from the sensing areas 2202 from the multi-analyte (e.g., glucose and oxidation), multi-site array to calculate glucose values.

In some aspects, the analyte sensor 100 may include one or more drug-eluting polymer matrices on all or a portion of an external surface of the sensor housing 102 (on one or more regions or cutouts 708 of the sensor housing 102). In some aspects, one or more therapeutic agents may be dispersed within the one or more drug eluting polymer matrices. In some aspects, the one or more therapeutic agents may reduce or stop the migration of neutrophils from entering the space in which the analyte sensor 100 has been implanted and, thus, reduce or stop the production of hydrogen peroxide and fibrotic encapsulation. Accordingly, in some aspects, the one or more therapeutic agents may reduce deterioration of the one or more indicator elements 106 (e.g., indicator elements 106a and 106b). In some aspects, the one or more therapeutic agents, which may be dispersed within the drug eluting polymer matrix, may include one or more anti-inflammatory drugs, such as, for example, non-steroidal anti-inflammatory drug (e.g., acetylsalicylic acid (aspirin) and/or isobutylphenylpropanoic acid (ibuprofen)). In some aspects, the one or more therapeutic agents dispersed within the drug-eluting polymer matrix may include one or more glucocorticoids. In some aspects, the one or more therapeutic agents may include one or more of dexamethasone, triamcinolone, betamethasone, methylprednisolone, beclometasone, fludrocortisone, derivatives thereof, and analogs thereof. In some aspects, the one or more therapeutic agents may reduce the production of hydrogen peroxide by neutrophils and macrophages.

In some aspects, as shown in FIG. 1, the analyte sensor 100 may communicate directly with the external transceiver 101 and/or the display device 105. In some aspects, the analyte sensor 100 may receive commands from the transceiver 101 and/or the display device 105, and the analyte sensor 100 may convey measurement data from the sensing devices 100A and/or 100B (e.g., from the measurement electronics of the sensing areas 2202a, 2202c, 2202b, and 2202d of the analyte sensor 100). In some aspects, the measurement data may include one or more readings from photodetectors 224, 226, 228, and/or 230 of the sensing devices 100A and/or 100B and/or one or more readings from one or more temperature transducers of the sensing devices 100A and 100B. In some aspects, the transceiver 101 and/or display device 105 may use the measurement data received from the analyte sensor 100 to calculate analyte concentrations. In some alternative aspects, the analyte sensor 100 may use the measurement data to calculate analyte concentrations and may convey the calculated analyte concentrations (in addition to or as an alternative to conveying the measurement data).

In some aspects, the transceiver 101 and/or display device 105 may implement a passive telemetry for communicating with the analyte sensor 100 via an inductive magnetic link for power and/or data transfer. In some aspects, as shown in FIGS. 2 and 3, the analyte sensor 100 may include an antenna 114, which may be, for example, a ferrite-based micro-antenna. In some aspects, as shown in FIGS. 2 and 3, the antenna 114 may be an inductor including a conductor 702 in the form of a coil and a magnetic core 704. In some aspects, the core 704 may be, for example and without limitation, a ferrite core. In some aspects, as illustrated in FIGS. 2 and 3, the substrates 112a and 112b of the sensing devices 100A and 100B may be attached to the antenna 114. In some aspects, the circuit components 111 of the substrates 112 may be connected electrically to the antenna 114.

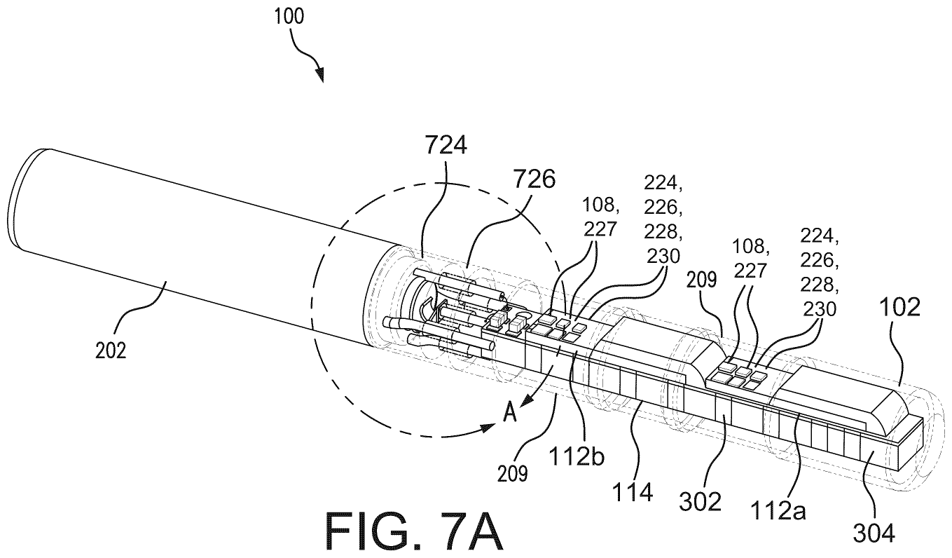

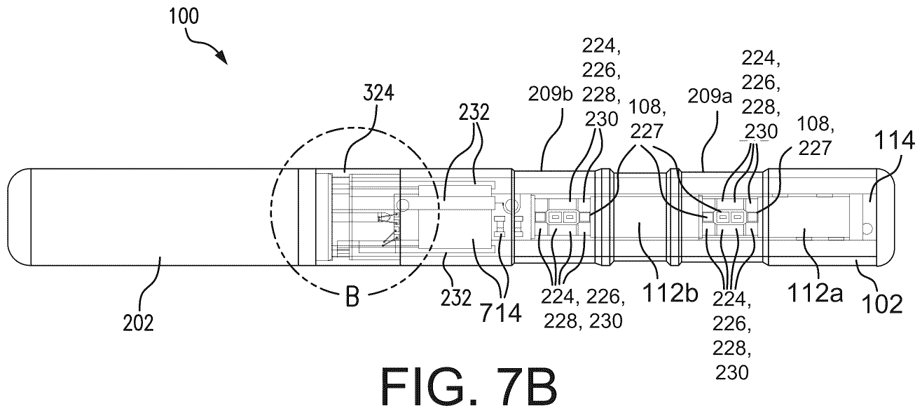

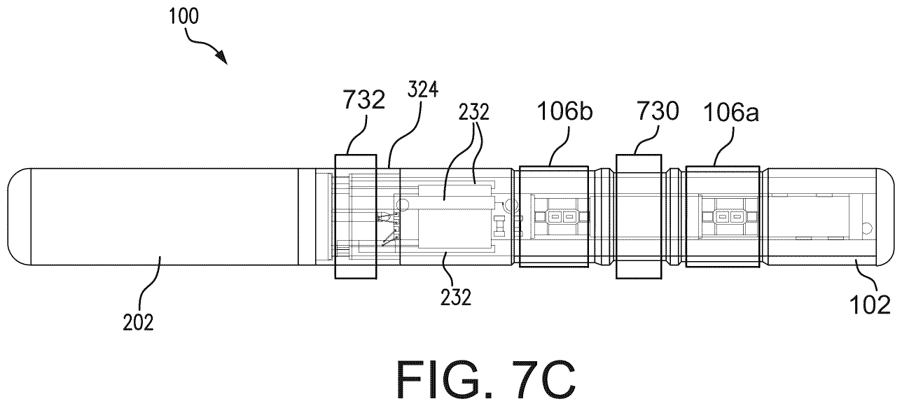

In some aspects, the analyte sensor 100 may not include a charge storage device (e.g., battery), and, as a result, the analyte sensor 100 may rely on the transceiver 101 and/or the display device 105 to provide power for the analyte sensor 100 and a data link to convey analyte-related data from the analyte sensor 100 to the transceiver 101 and/or the display device 105. However, this is not required, and, in some alternative aspects, as described below with reference to FIGS. 7A-7C, the analyte sensor 100 may include a charge storage device (e.g., a battery).

In some aspects (e.g., some aspects in which the analyte sensor 100 does not include a charge storage device), the analyte sensor 100 may be a passive, fully implantable multisite sensing system having a small size. For an analyte sensor 100 that is a fully implantable sensing system having no battery power source, the transceiver 101 and/or the display device 105 may provide energy to run the analyte sensor 100 via a magnetic field. In some aspects, the magnetic link the may provide energy and a link for data transfer using amplitude modulation (AM). Although in some aspects, data transfer is carried out using AM, in alternative aspects, other types of modulation may be used. In some aspects, the transceiver 101 and/or the display device 105 may communicate with and/or power the analyte sensor 100 using near field communication (e.g., at a frequency of 13.56 MHz, which can achieve high penetration through the skin and is a medically approved frequency band). However, this is not required, and, in other aspects, different frequencies may be used for powering and communicating with the analyte sensor 100.

In some alternative aspects, as shown in FIGS. 7A-7C, the analyte sensor 100 may include a charge storage device 202. FIG. 7A is a perspective view of an implantable analyte sensor 100 of the system 50 according to some charge storage device aspects. FIGS. 7B and 7C are side views of an implantable analyte sensor 100 of the system 50 according to some alternative charge storage device aspects. Relative to FIG. 7B, FIG. 7C additionally shows one or more indicator elements 106 (e.g., first indicator element 106a and second indicator element 106b) and one or more drug eluting polymer matrices 730 and 732, which may be part of the analyte sensor 100 (e.g., in or on portions of the exterior surface of the sensor housing 102 of the analyte sensor 100). In some aspects, as shown in FIGS. 7A-7C, the implantable analyte sensor 100 that includes the charge storage device 202 may also include the antenna 114 (e.g., an inductive element), the one or more substrates 112 (e.g., the first substrate 112a and the second substrate 112b), the sensor housing 102, the first light sources 108, the second light sources 227, the photodetectors 224, 226, 228, and/or 230, the one or more indicator elements 106 (e.g., the first and second indicator elements 106a and 106b) and the one or more drug eluting polymer matrices (e.g., drug eluting polymer matrices 730 and 732).

In some aspects, circuitry of the analyte sensor 100 may include the antenna 114, the circuit components mounted on or fabricated in the one or more substrates 112 (e.g., the one or more first light sources 108, the one or more second light sources 227, the photodetectors 224, 226, 228, and/or 230, the one or more temperature transducers, and/or the circuit components 111), and/or one or more circuit components (e.g., the circuit components 714 shown in FIG. 7B) mounted to the antenna 114. In some aspects, the circuitry of the analyte sensor 100 may be powered by the charge storage device 202.

In some aspects, the charge storage device 202 may be a battery (e.g., a rechargeable battery such as a lithium-ion battery or a non-rechargeable battery), a capacitor, or a super capacitor. In some aspects, at least the exterior of the charge storage device 202 may be made of a biocompatible material such as, for example and without limitation, stainless steel or a titanium alloy. In some aspects, the charge storage device 202 may include a positive terminal (cathode) and a negative terminal (anode).

In some aspects, as shown in FIGS. 7A-7C, one or more couplers may attach the charge storage device 202 to the sensor housing 102. In some aspects, as shown in FIG. 7A, the one or more couplers that attach the charge storage device 202 to the housing 102 may include a power source terminal enclosure 724 and a housing cap enclosure 726. In some aspects, electrically conductive connectors may electrically connect the positive and negative terminals, respectively, of the charge storage device 202 to the circuitry of the analyte sensor 100. In some aspects, the attachment of the charge storage device 202 to the housing 102 may be supported by one or more supports. In some aspects, as shown in FIG. 2A, the circuitry of the analyte sensor 100 may extend away from the charge storage device 202 along the longitudinal axis of the charge storage device 202.

In some alternative aspects, as shown in FIG. 7B, a coupler 324 may attach the housing 102 and the charge storage device 202. In some aspects, the coupler 324 may be between the housing 102 and the charge storage device 202. In some aspects, as shown in FIGS. 7B, the coupler 324 may include one or more supports 232 (e.g., reinforcement rods, bars, or beams), which may be attached to and/or integral with the coupler 324. In some aspects, the analyte sensor 100 including the charge storage device 202, the coupler 324, and the housing 102 may be hermetically sealed. In some aspects, the analyte sensor 100 may include first and second electrically conductive connectors, which connect the positive and negative terminals, of the charge storage device 202 to the circuitry of the analyte sensor 100. In some aspects, as shown in FIG. 7B, the circuitry of the analyte sensor 100 may extend away from the charge storage device 202 along the longitudinal axis of the charge storage device 202.

In some aspects, as shown in FIG. 7C, the analyte sensor 100 may include one or more drug-eluting polymer matrices. In some aspects, the analyte sensor 100 may include one or more drug-eluting polymer matrices (e.g., the drug-eluting polymer matrix 730) on all or a portion of the external surface of the housing 102. In some aspects, the one or more drug-eluting polymer matrices on the housing 102 may be located in one or more recesses in the housing 102. In some aspects, the analyte sensor 100 may additionally or alternatively include one or more drug-eluting polymer matrices (e.g., the drug-eluting polymer matrix 732), on all or a portion of an external surface of the one or more couplers attaching the charge storage device 202 and the housing 102. In some aspects, one or more drug-eluting polymer matrices may be located on all or a portion of one or both of the power source terminal enclosure 724 and the housing cap enclosure 726 shown in FIG. 7A. In some aspects, as shown in FIG. 7C, one or more drug-eluting polymer matrices 332 may be located on all or a portion of the coupler 324.

In some aspects, the one or more drug-eluting polymer matrices may be applied to the sensor housing 102 and/or one or more couplers (e.g., the coupler 324 or power source terminal enclosure 724 and the housing cap enclosure 726) via dip or spray coating. In some alternative aspects, the one or more drug-eluting polymer matrices may have a pre-formed shape such as, for example, a ring or sleeve. In some alternative aspects, the one or more drug-eluting polymer matrices may have a different shape. In some aspects, as shown in FIG. 7C, the one or more one or more drug-eluting polymer matrices 730 and 732 may wrap around a portion of the sensor housing 102 and/or a portion of the coupler 324. In some alternative aspects, the one or more drug-eluting polymer matrices 730 and 732 may be wider or narrower than the drug-eluting polymer matrices 730 and 732 illustrated in FIG. 7C.

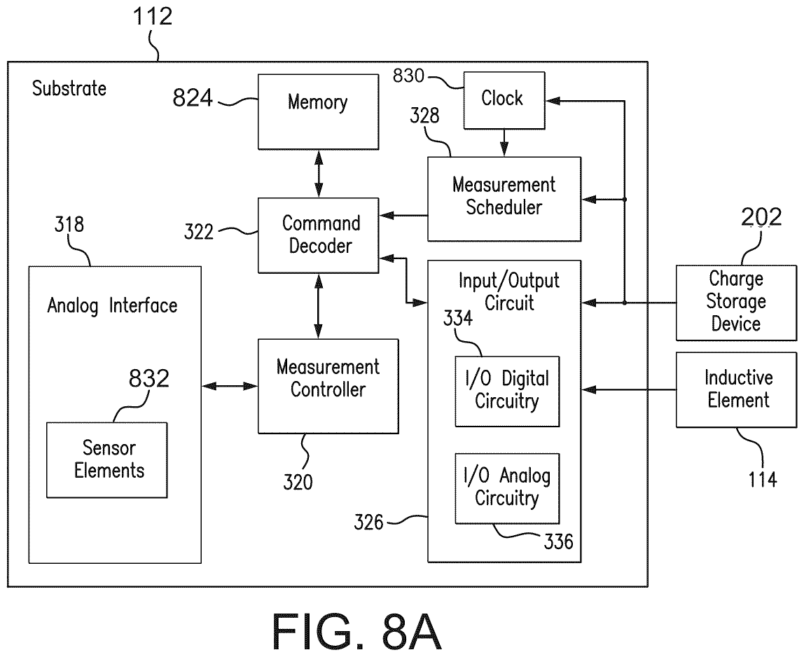

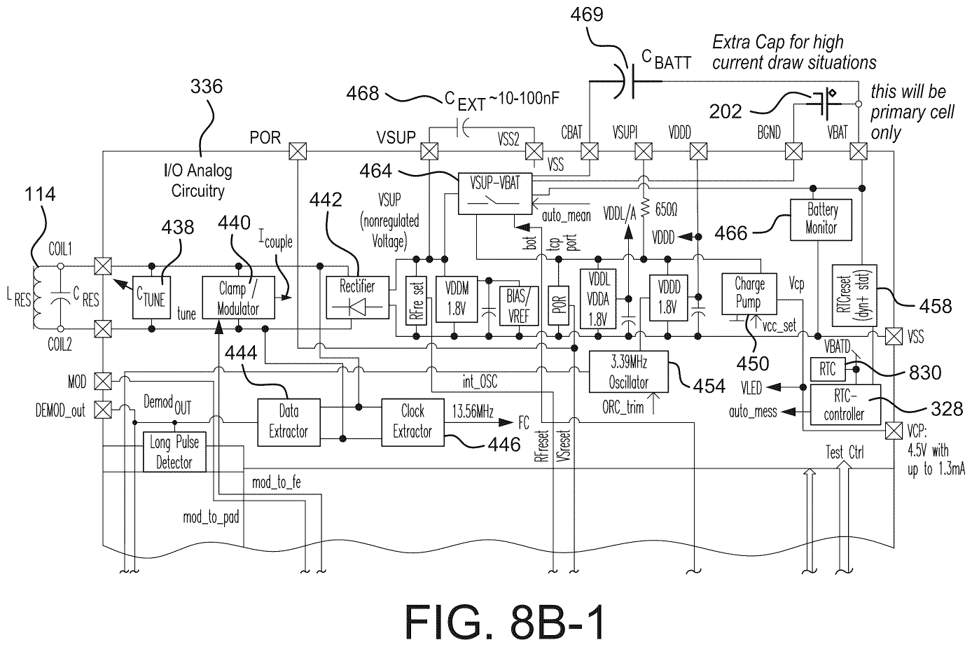

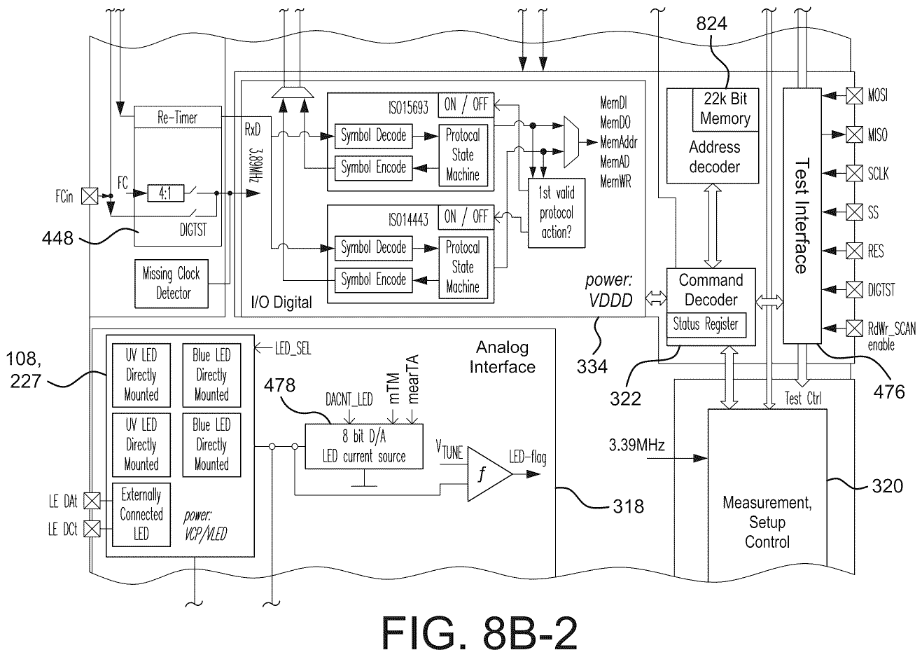

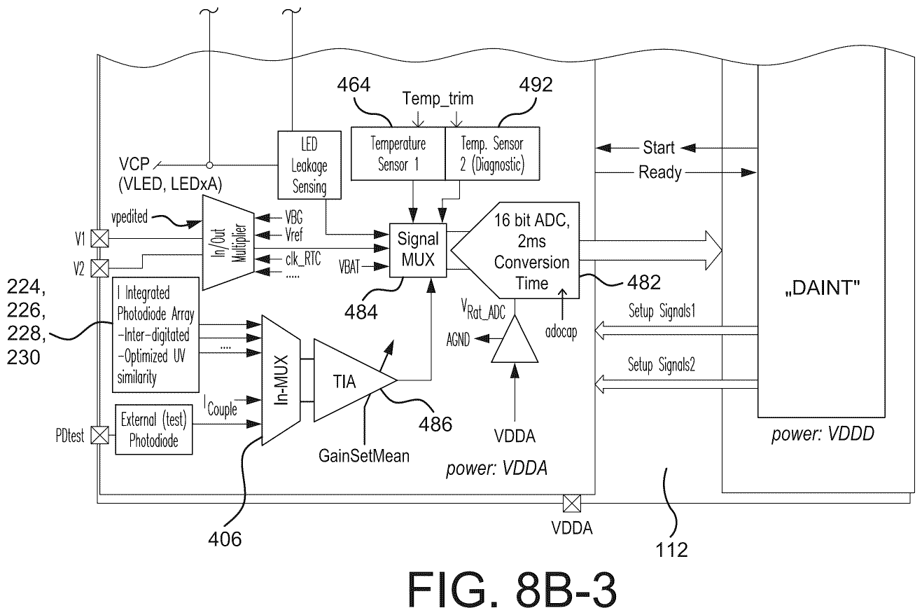

FIG. 8A is a block diagram illustrating the main functional blocks of the circuitry of a sensing device (e.g., sensing device 100A or 100B) of an analyte sensor 100 including a charge storage device 202 and embodying aspects of the present invention. In some aspects, as illustrated in FIG. 8A, the circuitry of a sensing device may include the circuit components 111 mounted on or fabricated in a substrate 112 (e.g., the first substrate 112a and/or the second substrate 112b) of the sensor 100, which may include one or more of an analog interface 318, a measurement controller 320, a command decoder 322, a memory 824, an input/output (I/O) circuit 326, a measurement scheduler 328, and a clock 830. In some aspects, the analog interface 318 may include one or more sensor elements 832 mounted on or fabricated in the substrate 112 (e.g., the first substrate 112a and/or the second substrate 112b). In some aspects, the sensor elements 832 may include, for example, the one or more first light sources 108, the one or more second light sources 227, the photodetectors 224, 226, 228, and/or 230, and/or the one or more temperature transducers. In some aspects, the sensor 100 may alternatively or additionally have one or more sensor elements external to the substrate(s) 112 (i.e., sensor elements that that are neither mounted on nor fabricated in the first and second substrates 112a and 112b) but electrically connected to the analog interface 318 via one or more contacts. In some aspects, the analog interface 318 may include one or more light source drivers, one or more amplifiers, one or more analog-to-digital convertors (ADCs), one or more comparators, and/or one or more multiplexors.

In some aspects, the I/O circuit 326 may include I/O digital circuitry 334 and/or I/O analog circuitry 336. In some aspects, the antenna 114 may be electrically connected to the I/O circuit 326, which may use current flowing through the antenna 114 to generate power for the sensor 100 and/or to extract data from the current. In some aspects, the I/O circuit 326 may also convey data (e.g., to the transceiver 101 and/or display device 105) by modulating the current the flowing through the antenna 114. In some aspects, the I/O circuit 326 may be electrically connected to and be powered by the charge storage device 202 (e.g., at least during times when the sensor 100 is not receiving power from an external device such as the transceiver 101 or the display device 105). In some aspects in which the analyte sensor 100 include multiple sensing devices, although not shown in FIG. 8A, the antenna 114 may be electrically connected to the circuitry of the multiple sensing devices.

In some aspects, the charge storage device (CSD) 202 may provide power to the clock 830 and to the measurement scheduler 328. In some aspects, the CSD-powered clock 830 may provide a continuous clock for driving circuitry of the sensor 100 even when the sensor 100 is not receiving power from an external device (e.g., the transceiver 101 and/or the display device 105). In some aspects, the measurement scheduler 328 may use the continuous clock output of the clock 830 to keep track of time and initiate autonomous, self-powered analyte measurements when appropriate (e.g., at periodic intervals, such as, for example, every minute, every two minutes, every 5 minutes, every 10 minutes, every 15 minutes, every half-hour, every hour, every two hours, every six hours, every twelve hours, or every day). The autonomous analyte measurements may be stored in the memory 824. In some aspects, the I/O circuit 326 may convey one or more of the stored measurements to the external device (e.g., the transceiver 101 and/or the display device 105) at a later time. For example, in some request aspects, the I/O circuit 326 may convey one or more of the stored measurements in response to the analyte sensor 100 receiving and decoding a measurement data request from the transceiver 101 and/or the display device 105. In some alternative trigger aspects, the I/O circuit 326 may convey one or more of the stored measurements in response to detecting that the transceiver 101 and/or display device 105 is present (e.g., when an electrodynamic field generated by the transceiver 101 and/or display device 105 induces a current in the antenna 114 of the analyte sensor 100). In some aspects in which the analyte sensor 100 include multiple sensing devices, although not shown in FIG. 8A, the CSD 202 may be electrically connected to the circuitry of the multiple sensing devices.

In some aspects, the memory 824 may be a nonvolatile storage medium. In some aspects, the memory 824 may be an electrically erasable programmable read only memory (EEPROM). However, in some alternative aspects, other types of nonvolatile storage media, such as flash memory, may be used. In some aspects, the memory 824 may be a 20 by a 22kBit memory, but this is not required, and, in some alternative aspects, the memory 824 may be a different size. In some aspects, the memory 824 may include an address decoder. In some aspects, the memory 824 may store measurement information autonomously generated while the sensor 100 is powered from the charge storage device 202. In some aspects, the memory 824 may additionally or alternatively store one or more time-stamps identifying when the measurement data was generated, sensor calibration data, a unique sensor identification, setup information, and/or integrated circuit calibration data. In some aspects, the unique identification information may, for example, enable full traceability of the sensor 100 through its production and subsequent use. In some aspects, the memory 824 may receive write data (i.e., data to be written to the memory 824) from the command decoder 322 and may supply read data (i.e., data read from the memory 824) to the command decoder 322. In some aspects, memory 824 may have an integrated charge pump and/or may be connected to an external charge pump.

In some aspects, the measurement scheduler 328 may issue an autonomous measurement command (e.g., to the command decoder 322, which may decode the command and/or send the command to the measurement controller 320, or directly to the measurement controller 320. The measurement controller 320 may control the sensor elements 832 of the analog interface 318 to perform an autonomous analyte measurement sequence, and the results of the autonomous analyte measurement may be stored in the memory 824. In some alternative aspects, instead of issuing an autonomous measurement command that is decoded by the command decoder 322, the measurement scheduler 328 may communicate with the measurement controller 320 initiate the performance of the autonomous analyte measurement sequence. In some aspects, the autonomous measurement command may be a control signal that changes a state (e.g., from low to high) to initiate the performance of the autonomous analyte measurement sequence. In some further alternative aspects, the functionality of the measurement scheduler 328 may be included in the measurement controller 320, and, in these aspects, the measurement controller 320 may use the clock 830 to determine when to perform the autonomous analyte measurement sequence.

FIGS. 8B-1, 8B-2, and 8B-3 show sections of a block diagram illustrating the functional blocks of circuitry mounted on or fabricated in the substrate 112 according to some aspects. In some aspects, as shown in FIG. 8B-1, the antenna 114, which may be in the form of a coil, may be external to the substrate 112 and may be connected to the I/O analog circuitry 336 through contacts COIL1 and COIL2. In some aspects in which the analyte sensor 100 include multiple sensing devices, although not shown in FIGS. 8B-1, 8B-2, and 8B-3, the antenna 114 may be electrically connected to the circuitry of the multiple sensing devices.

In some aspects, as shown in FIG. 8B-1, the I/O analog circuitry 336 may include one or more of a capacitor 438, clamp/modulator 440, a rectifier 442, a data extractor 444, a clock extractor 446, a frequency divider 448, a charge pump 450, and an oscillator 454. In some aspects, one or more of the capacitor 438, clamp/modulator 440, rectifier 442, data extractor 444, and clock extractor 446 may be connected to the antenna 114 through one or more of contacts COIL1 and COIL2. The rectifier 442 may convert an alternating current produced by the antenna 114 to a direct current that may be used to power circuit components 111 of a sensing device of the analyte sensor 100. For example, the direct current may be used to produce one or more voltages, such as, for example, voltages VDDL or VDDA, which may be used to power the analog interface 318, and/or VDDD, which may be used to power one or more of the I/O digital circuit 336, the memory 824, the measurement controller 320, the command decoder 322, the measurement scheduler 328, and/or a test interface 476. In some aspects, the rectifier 442 may be a Schottky diode; however, other types of rectifiers may be used in some alternative embodiments. In some aspects, the data extractor 444 may extract data from the alternating current produced by the antenna 114. In some aspects, the clock extractor 446 may extract a signal having a frequency (e.g., 13.56 MHz) from the alternating current produced by the antenna 114. In some aspects, the frequency divider 448 may divide the frequency of the signal output by the clock extractor 446. For example, in some aspects, the frequency divider 448 may comprise a 4:1 frequency divider that receives a signal having a frequency (e.g., 13.56 MHz) as an input and outputs a signal having a frequency (e.g., 3.39 MHz) equal to one fourth the frequency of the input signal. In some aspects, the frequency divider 448 may output either the frequency divided output of the clock extractor 446 or the output of the oscillator 454 to the I/O digital circuitry 336. In some aspects, the outputs of rectifier 442 may be connected to one or more capacitors 468 (e.g., one or more regulation capacitors) through contacts VSUP and VSS.

In some aspects, as shown in FIG. 8B-1, the I/O analog circuitry 336 may include a charge pump 450. In some aspects, the charge pump 450 may produce a voltage VLED that is used to power the one or more light sources 108, 227. In some aspects, the charge pump 450 may additionally or alternatively produce a voltage of the charge pump (VCP).

In some aspects, as shown in FIG. 8B-1, the CSD 202 may be electrically connected to circuitry of the sensing device (e.g., via contacts VBAT and BGND). In some aspects in which the analyte sensor 100 include multiple sensing devices, although not shown in FIGS. 8B-1, 8B-2, and 8B-3, the CSD 202 may be electrically connected to the circuitry of the multiple sensing devices. In some aspects, the analyte sensor 100 may include a capacitor 469 connected to circuitry of the sensing device (e.g., via contacts VBAT and CBAT). In some aspects, the capacitor 469 may be for high current draw situations.

In some aspects, as shown in FIG. 8B-1, the I/O analog circuitry 336 may include a power switch 464. The power switch 464 may switch the sensor 100 between CSD power provided by the charge storage device 202 and externally supplied power provided by an external device (e.g., the transceiver 101 or the display device 105) via the antenna 114 and rectifier 442 of a sensing device of the sensor 100. In some aspects, the power switch 464 may switch circuit components of the sensing device of the sensor 100 from being powered by the voltage VSUP produced by the rectifier 442 using a current induced in the antenna 114 to being powered by the voltage VBAT produced by the charge storage device 202.

In some aspects, the power switch 464 may switch the sensing device of the sensor 100 to power itself from the power of the charge storage device 202 in response to an autonomous measurement command initiated by the measurement scheduler 328. For instance, in some aspects, the sensing device of the sensor 100 may be in a sleep mode while the sensor 100 is not receiving power from an external device (e.g., the transceiver 101 or the display device 105). In the sleep mode, no power would be supplied to at least a subset of the circuit components of the sensing device (e.g., one or more of the I/O digital circuitry 336, command decoder 322, memory 824, measurement controller 320, and analog interface 318). However, in some aspects, in the sleep mode, at least the clock 830 and measurement scheduler 328 would receive power from the charge storage device 202. The measurement scheduler 328 may use the CSD-powered clock 830 to determine when to initiate an autonomous measurement. In some aspects, in response to an autonomous measurement command from the measurement scheduler 328, the power switch 464 may switch sensing device of the sensor 100 to the power of the charge storage device 202. In some aspects, one or more of the I/O digital circuitry 336, command decoder 322, memory 824, measurement controller 320, and analog interface 318 would then be powered by the charge storage device 202. In some aspects, when the sensor 100 is switched to the power of the charge storage device 202, the voltage VBAT (instead of the voltage VSUP) may be used to produce the voltage (e.g., voltages VDDA, VDDD, and VLED) that powers the sensor 100. In this way, the measurement scheduler 328 can wake up the sensor 100 by issuing a measurement command that causes the power switch 464 to switch the sensor 100 to the power of the charge storage device 202.

In some aspects, as shown in FIG. 8B-1, the clock 830 may be a pseudo real time clock (RTC). In some aspects, as described above, a sensing device of the analyte sensor 100 may use the clock 830 to realize the sleep mode during which the sensing device is in a low power mode while the analyte sensing device waits to take another autonomous measurement. In some aspects, during the sleep/low power mode, the CSD 202 may power the clock 830 and the measurement scheduler 328 but may not provide power to the subset of the circuit components of the sensing device (e.g., one or more of the I/O digital circuitry 336, command decoder 322, memory 824, measurement controller 320, and analog interface 318). In some aspects, the number of clock cycles that the sensing device will wait during sleep period may be programmed into a rtc_ref_value in the memory 824. In some aspects, the frequency of the clock 830 may differ from ASIC die to ASIC die (e.g., in the range of 1.8 kHz-6.2 kHz), and the frequency of the clock 830 may be voltage and/or temperature dependent. That is, in some aspects, the frequency of the clock 830 may change based on the voltage supplied to the clock 830 (e.g., the voltage VBAT produced by the CSD 202), and the frequency of the clock 830 may additionally or alternatively change based on the temperature of the clock. In some aspects, at wafer level calibration, the nominal frequency of the clock 830 at a nominal temperature and a nominal voltage may be measured and stored (e.g., in units of Hz) in an RTC_freq value in the memory 824. In an example in which the one or more sensing devices of the analyte sensor 100 takes an autonomous measurement at a frequency of approximately every 5 minutes, if an RTC_freq value of 4,500 is stored in the memory 824, then rtc_ref_value may be programmed to 300*45,00=1,350,000 to have an autonomous measurement interval close to 5 minutes at the nominal temperature and voltage.

In some aspects, as shown in FIG. 8B-1, the I/O analog circuitry 336 may include a CSD monitor 466 configured to monitor the voltage VBAT produced by the charge storage device 202 and provide feedback about the charge level of the charge storage device 202. For instance, in some aspects, the CSD monitor 466 may indicate whether the voltage VBAT is sufficient for operation of the sensing device, and the power switch 464 may only switch the sensing device of sensor 100 to CSD power if the CSD monitor 466 indicates that the voltage VBAT is sufficient for sensor operation. In some aspects, the CSD monitor 466 may determine whether the voltage VBAT is sufficient for sensor operation by comparing the voltage VBAT to an operational threshold voltage. In some aspects, the measurement scheduler 328 may adjust the frequency at which autonomous measurements are taken based on the charge level of the charge storage device 202 as indicated by the CSD monitor 466. For instance, in some aspects, if the CSD monitor 466 indicates that the charge level of the charge storage device 202 is low, the measurement scheduler 328 may adjust the frequency at which autonomous measurements are taken.

In some aspects, as shown in FIG. 8B-2, the I/O digital circuitry 334 may include a decoder, an encoder, and a protocol state machine. The decoder may decode the data extracted by the data extractor 444 from the alternating current produced by antenna 114. The command decoder 322 may receive the data decoded by the decoder and may decode commands therefrom. In some aspects, the command decoder 322 may comprise a status register. In some aspects, the encoder may receive data from the command decoder 322 and encode the data. In some aspects, the I/O digital circuitry 336 may include two or more sets of encoders and decoders with each set having its own protocol state machine. In this way, the sensing device of the sensor 100 may be able to convey and receive information using more than one communication protocol. For example, in some aspects, as shown in FIG. 8B-2, the I/O digital circuitry 334 may include an ISO14443 decoder, encoder, and protocol state machine set and an ISO15693 decoder, encoder, and protocol state machine set.

In some aspects, as shown in FIGS. 8B-1 and 8B-2, the clamp/modulator 440 of the I/O analog circuitry 336 may receive the data encoded by the encoder and may modulate the current flowing through the antenna 114 as a function of the encoded data. In this way, the encoded data may be conveyed wirelessly by the antenna 114 as a modulated electromagnetic wave. The conveyed data may be detected by an external reading device (e.g., the transceiver 101 and/or display device 105) by, for example, measuring the current induced by the modulated electromagnetic wave in a coil of the external reading device. Furthermore, by modulating the current flowing through the antenna 114 as a function of the encoded data, the encoded data may be conveyed wirelessly by the antenna 114 as a modulated electromagnetic wave even while the antenna 114 is being used to produce operating power for the sensor 100. In some aspects, the communications received by the antenna 114 and/or the communications conveyed by the antenna 114 may be radio frequency (RF) communications. Although, in the illustrated aspect, the sensor 100 includes a single antenna 114, some alternative aspects of the sensor 100 may include two or more inductive elements (e.g., one coil for data conveyance and one coil for power and data reception).

In some aspects, as shown in FIGS. 8B-2 and 8B-3, the analog interface 318 may include a current source 478, one or more light source drivers 480, an analog to digital converter (ADC) 482, a signal multiplexer (MUX) 484, a comparator 486, one or more photodetectors 488 (e.g., photodetectors 224 and 226), and/or one or more temperature transducers 464 and 492. In some non-limiting embodiments, the comparator 486 may be a transimpedance amplifier (TIA). However, this is not required, and, in some alternative aspects, the comparator 486 may be a different type of comparator. In some aspects, one or more of the temperature transducers 464 and 492 may be a band-gap based temperature transducer. However, in some alternative embodiments, different types of temperature transducers may be used, such as, for example, thermistors or resistance temperature detectors. In some aspects, the analog interface 318 may include two temperature transducers 464 and 492 for high reliability operation and for detection of temperature error/failure with higher probability. In some aspects, the second temperature transducer 492 may be a redundant temperature transducer that is the same as the first temperature transducer 464 and may be for temperature plausibility/diagnostic purposes. In some aspects, the one or more temperature transducers 464 and 492 may be fabricated in the substrate 112 or mounted on the semiconductor substrate 112. The one or more temperature transducers 464 and 492 may output an analog temperature measurement signal indicative of the temperature of the sensor 100.

In some aspects, as shown in FIG. 8B-3, the one or more photodetectors 224, 226, 228, 230 may be fabricated in or mounted on the substrate 112. In some aspects, the one or more photodetectors 224, 226, 228, 230 may include a photodetector array including, for example, eight photodetectors. In some aspects, one or more of the photodetectors 224, 226, 228, 230 may be coated with one or more optical filters.

In some aspects, as shown in FIG. 8B-2, the one or more light source drivers 480 may drive the one or more light sources 108, 227 using current provided by the current source 478. In some aspects, the one or more light sources 108 of the sensor 100 may include a first light source 108 (e.g., a UV light source) and a second light source 227 (e.g., a blue light source). In some aspects, as illustrated in FIG. 8B-2, the first and second light sources 108, 227 may be mounted to the substrate 112 and connected to the substrate 112 via contacts. However, this is not required, and, in some alternative aspects, one or more of the first and second light sources 108, 227 may be fabricated in the substrate 112. In some aspects, the one or more light sources may be powered using a voltage VLED generated using the charge pump 450. In some aspects, the one or more light source drivers 480 may receive a light source selection signal from the measurement controller 320 that identifies which of the one or more light sources 108, 227 should be driven by the one or more light source drivers 480.

In some aspects, the current source 478 may receive a signal from the measurement controller 320 indicating the light source current at which a light source 108, 227 is to be driven, and the current source 478 may provide a current accordingly. The one or more light sources 108, 227 may emit radiation from an emission point in accordance with one or more drive signals from the one or more light source drivers 480. The one or more photodetectors 224, 226, 228, 230 may each output an analog light measurement signal indicative of the amount of light received by the photodetector.

In some aspects, as shown in FIG. 8B-3, the analog interface 318 may include an input multiplexor 406. The input multiplexor 406 may receive the analog light measurement signals outputted by the one or more photodetectors 224, 226, 228, 230. In some aspects, under the control of the measurement controller 320, the input multiplexor 406 may select one or two of the analog light measurement signals to pass through to the comparator 486. In some aspects, the comparator 486 may amplify and/or compare the one or more analog light measurement signals received from the input multiplexor 406.

In some aspects, as shown in FIG. 8B-3, the signal MUX 484 may receive one or more analog temperature measurement signals from the one or more temperature transducers 464 and 492, one or more analog light measurement signals from the one or more photodetectors 224, 226, 228, 230, an analog light difference measurement signal from the comparator 486, and/or one more analog voltage measurements signals from the CSD monitor 466. In some aspects, under the control of the measurement controller 320, the signal MUX 484 may select one of the received signals and output the selected signal to the ADC 482. The ADC 482 may receive the selected analog signal from the signal MUX 484, convert the received analog signal to a digital signal, and supply the digital signal to the measurement controller 320. In this way, the ADC 482 may convert the one or more analog temperature measurement signals, the one or more analog light measurement signals, and/or the analog light difference measurement signal, and/or the one or more analog short term measurements to one or more digital temperature measurement signals, one or more digital light measurement signals, and/or a digital light difference measurement signal, respectively. In some aspects, the ADC 482 may supply the digital signals, one at a time, to the measurement controller 320. In some aspects, the ADC 482 may be a 16 bit ADC, and the ADC 482 may have, for example, a 2 ms conversion time. However, this is not required, and some alternative aspects may use a different ADC.

In some aspects, the circuitry of a sensing device of the sensor 100 may include a field strength measurement circuit. In some aspects, the field strength measurement circuit may be part of the I/O analog circuitry 336, I/O digital circuitry 334, or the measurement controller 320, or the field strength measurement circuit may be a separate functional component. The field strength measurement circuit may measure the received (i.e., coupled) power (e.g., in mWatts). The field strength measurement circuit of the sensor 100 may produce a coupling value proportional to the strength of coupling between the antenna 114 of the sensor 100 and an antenna of an external device (e.g., transceiver 101 and/or display device 105). For example, in some aspects, the coupling value may be a current or frequency proportional to the strength of coupling.

In some aspects, as illustrated in FIG. 8B-1, the clamp/modulator 440 of the I/O analog circuitry 336 acts as the field strength measurement circuit by providing a value (e.g., Icouple) proportional to the field strength. In some aspects, as shown in FIG. 8B-3, the field strength value Icouple may be provided as an input to the signal MUX 484 (e.g., via the input MUX 406). When selected, the signal MUX 484 may output the field strength value Icouple to the ADC 482. The ADC 482 may convert the field strength value Icouple received from the signal MUX 484 to a digital field strength value signal and supply the digital field strength signal to the measurement controller 320. In this way, the field strength measurement may be made available to the measurement controller 320 (e.g., for determining whether the field strength is sufficient to carry out a measurement sequence).

In some aspects, as shown in FIG. 8B-2, a test interface 476 may be mounted on or fabricated in the substrate 112. In some aspects, the test interface 476 may enable wafer-level production testing of the substrate 112. In some aspects, the test interface 476 may be an SPI-taped interface (i.e., a wireless communication interface). In some aspects, the test interface 476 may receive signals via one or more contacts and may output signals via one or more contacts. The test interface 476 may communicate with the measurement controller 320 via the command decoder 322.

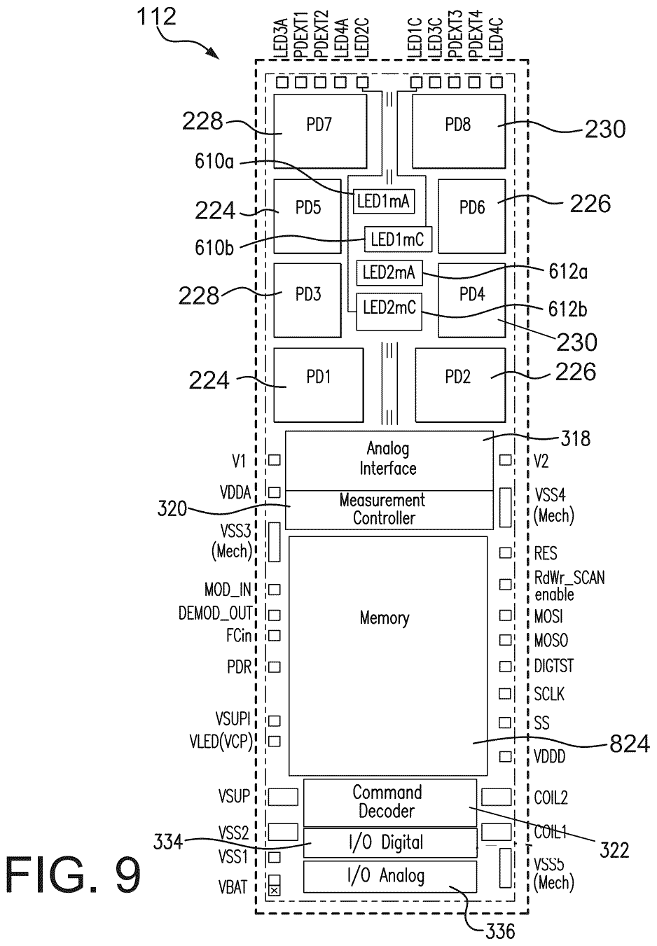

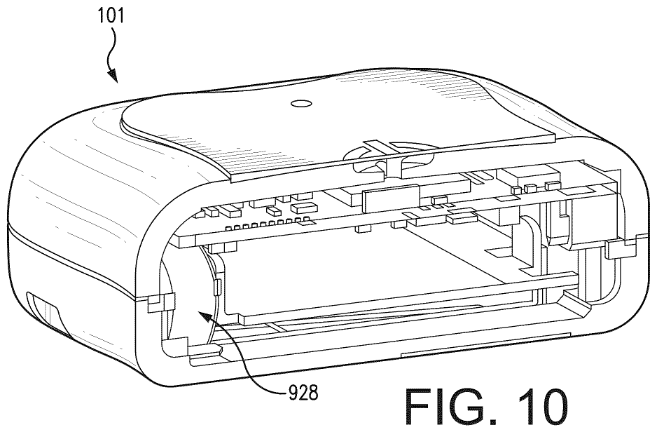

FIG. 9 illustrates the layout of a substrate 112 (e.g., first substrate 112a or second substrate 112b) according to some aspects (e.g., some aspects in which the substrate 112 is a semiconductor substrate). In some aspects, the substrate 112 may have a length of approximately 6010 μm and a width of approximately 1610 μm. However, this is not required, and, in some alternative aspects, the substrate 112 may have a different length and/or a different width. In some aspects, as shown in FIG. 9, eight photodetectors (e.g., two signal photodetectors 224, two reference photodetectors 226, two interferent photodetectors 228, and/or two second reference photodetectors 230) may be mounted on and/or fabricated in the substrate 112. In some aspects, as shown in FIG. 9, the substrate 112 may have light source mounting pads 610a, 610b, 612a, and 612b for mounting one or more first light sources 108 (e.g., one or more UV light sources) and/or one or more second light sources 227 (e.g., one or more blue light sources). However, this is not required, and, in some alternative aspects, the substrate 112 may have a different number of photodetectors fabricated therein, the photodetectors may be mounted on the substrate 112 instead of fabricated therein, the substrate may have a different number of light source mounting pads (e.g., mounting pads for one, three, or four light sources), and/or the light sources 108 and/or 227 may be fabricated in the substrate 112 instead of mounted thereon. In some aspects, the light source mounting pads 610a, 610b, 612a, and 612b may connect to the anodes and cathodes of light sources 108 and/or 227 mounted on the substrate 112.

In some aspects, as shown in FIG. 9, each of the substrates 112 of the analyte sensor 100 may include (i) a first set of one or more first light sources 108, one or more second light sources 227, one or more signal photodetectors 224, one or more reference photodetectors 226, one or more interferent photodetectors 228, and/or one or more second reference photodetectors 230 mount on and/or fabricated in the substrate 112 for one sensing area 2202 and (ii) a second set of one or more first light sources 108, one or more second light sources 227, one or more signal photodetectors 224, one or more reference photodetectors 226, one or more interferent photodetectors 228, and/or one or more second reference photodetectors 230 mount on and/or fabricated in the substrate 112 for another sensing area 2202.

In some aspects, the photodetectors 224, 226, 228, and 230 may be symmetrically formed on each side of a center line of the substrate 112. In some aspects, the light source mounting pads 610a, 610b, 612a, and 612b may be configured such that the emission points of light sources 108 and/or 227, when mounted on the light source mounting pads 610a, 610b, 612a, and 612b, are aligned on the center line running between the photodetectors 224, 226, 228, and 230. Similarly, in some aspects in which the light sources 108 and/or 227 are fabricated in the substrate 112, the emission points of the fabricated light sources 108 and/or 227 are aligned on the center line running between the photodetectors 224, 226, 228, and 230. In some aspects, the fabrication of symmetrical photodetectors 224, 226, 228, and 230 (i.e., photodetectors 224, 226, 228, and 230 which are symmetrical relative to the light source emission points) may realize dual channels that are closer to being identical to each other than can be achieved by using discrete parts (e.g., photodetectors mounted on the semiconductor substrate 112). The nearly identical photodetector channels may improve the accuracy of the sensor measurements. This may be especially true when, in some aspects, the nearly identical dual photodetector channels are utilized as a signal channel and a reference channel, respectively.

In some aspects, as illustrated in FIG. 9, the photodetectors 224, 226, 228, and 230 may surround the light source mounting pads 610a, 610b, 612a, and 612b. In some aspects, the photodetectors 224, 226, 228, and 230 above and below the light source mounting pads 610a, 610b, 612a, and 612b may be larger than the photodetectors 224, 226, 228, and 230 to the left and right of the light source mounting pads 610a, 610b, 612a, and 612b. However, this is not required, and, in some alternative aspects, all of the photodetectors 224, 226, 228, and 230 may have the same size.

The layout of the photodetectors 224, 226, 228, and 230 on the semiconductor (e.g., silicon) substrate 112 is not limited to the aspect illustrated in FIG. 4 or 9. One or more alternative aspects may use different photodetector layouts. In some aspects, as shown in FIG. 9, one or more circuit components (e.g., analog interface 318, measurement controller 320, memory 824, command decoder 322, and/or the I/O digital circuitry 334 and/or I/O analog circuitry 336 of the I/O circuit 326) may be mounted on or fabricated in the substrate 112 (e.g., in the layout shown in FIG. 9 or in a different layout).

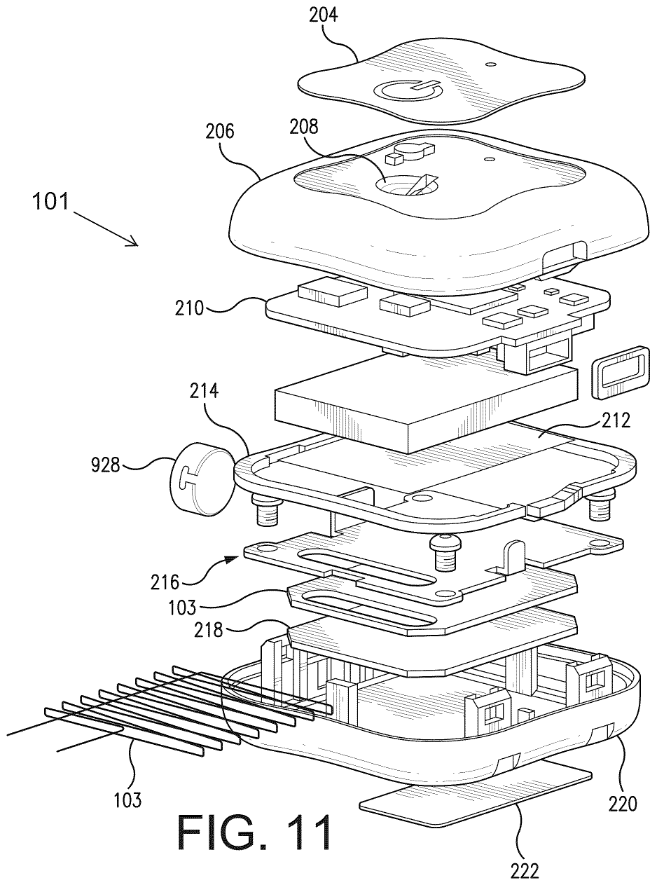

FIGS. 10-12 are perspective, exploded, and block diagrams of a transceiver 101 of the analyte monitoring system 50 according to some aspects. In some aspects, as shown in FIG. 11, the transceiver 101 may include an antenna 103, which may be, for example, an inductive element such as a coil. The transceiver 101 may generate an electromagnetic wave or electrodynamic field (e.g., by using an antenna 103) to induce a current in an antenna 114 of the analyte sensor 100, which may power and/or enable communication with the analyte sensor 100. In some aspects, the transceiver 101 may additionally or alternatively convey data (e.g., commands and/or data requests) to the analyte sensor 100. For example, in some aspects, the transceiver 101 may convey data by modulating the electromagnetic wave used to power the analyte sensor 100 (e.g., by modulating the current flowing through a coil of the transceiver 101). The modulation in the electromagnetic wave generated by the transceiver 101 may be detected/extracted by the analyte sensor 100. Moreover, the transceiver 101 may receive data (e.g., measurement information) from the analyte sensor 100. For example, in some aspects, the transceiver 101 may receive data by detecting modulations in the electromagnetic wave generated by the analyte sensor 100, e.g., by detecting modulations in the current flowing through the antenna 103 of the transceiver 101.

Although in some aspects, as illustrated in FIG. 1, the analyte sensor 100 may be a fully implantable sensor, this is not required, and, in some alternative aspects, the analyte sensor 100 may be a transcutaneous sensing system having a wired connection to the transceiver 101. For example, in some alternative aspects, the analyte sensor 100 may be located in or on a transcutaneous needle (e.g., at the tip thereof). In these aspects, instead of wirelessly communicating using antennas 103 and 114, the analyte sensor 100 and transceiver 101 may communicate using one or more wires connected between the transceiver 101 and the transceiver transcutaneous needle that includes the analyte sensor 100. For another example, in some alternative aspects, the analyte sensor 100 may be located in a catheter (e.g., for intravenous blood glucose monitoring) and may communicate (wirelessly or using wires) with the transceiver 101.

In some aspects, the analyte sensor 100 may include an interface device. In some aspects, the interface device may include the antenna 114 (e.g., inductive element) of the analyte sensor 100. In some of the transcutaneous aspects where there exists a wired connection between the analyte sensor 100 and the transceiver 101, the interface device may include the wired connection.

As illustrated in FIG. 11, in some aspects, the transceiver 101 may include a graphic overlay 204, front housing 206, button 208, printed circuit board (PCB) assembly 210, battery 212, gaskets 214, antenna 103, frame 218, reflection plate 216, back housing 220, ID label 222, and/or vibration motor 928. In some non-limiting aspects, the vibration motor 928 may be attached to the front housing 206 or back housing 220 such that the battery 212 does not dampen the vibration of vibration motor 928. In a non-limiting aspect, the transceiver electronics may be assembled using standard surface mount device (SMD) reflow and solder techniques. In one aspect, the electronics and peripherals may be put into a snap together housing design in which the front housing 206 and back housing 220 may be snapped together. In some aspects, the full assembly process may be performed at a single external electronics house. However, this is not required, and, in alternative aspects, the transceiver assembly process may be performed at one or more electronics houses, which may be internal, external, or a combination thereof. In some aspects, the assembled transceiver 101 may be programmed and functionally tested. In some aspects, assembled transceivers 101 may be packaged into their final shipping containers and be ready for sale.

In some aspects, as illustrated in FIGS. 10 and 11, the antenna 103 may be contained within the housing 206 and 220 of the transceiver 101. In some aspects, the antenna 103 in the transceiver 101 may be small and/or flat so that the antenna 103 fits within the housing 206 and 220 of a small, lightweight transceiver 101. In some aspects, the antenna 103 may be robust and capable of resisting various impacts. In some aspects, the transceiver 101 may be suitable for placement, for example, on an abdomen area, upper-arm, wrist, or thigh of a patient body. In some non-limiting aspects, the transceiver 101 may be suitable for attachment to a patient body by means of a biocompatible patch. Although, in some aspects, the antenna 103 may be contained within the housing 206 and 220 of the transceiver 101, this is not required, and, in some alternative aspects, a portion or all of the antenna 103 may be located external to the transceiver housing. For example, in some alternative aspects, antenna 103 may wrap around a user's wrist, arm, leg, or waist such as, for example, the antenna described in U.S. Pat. No. 8,073,548, which is incorporated herein by reference in its entirety.

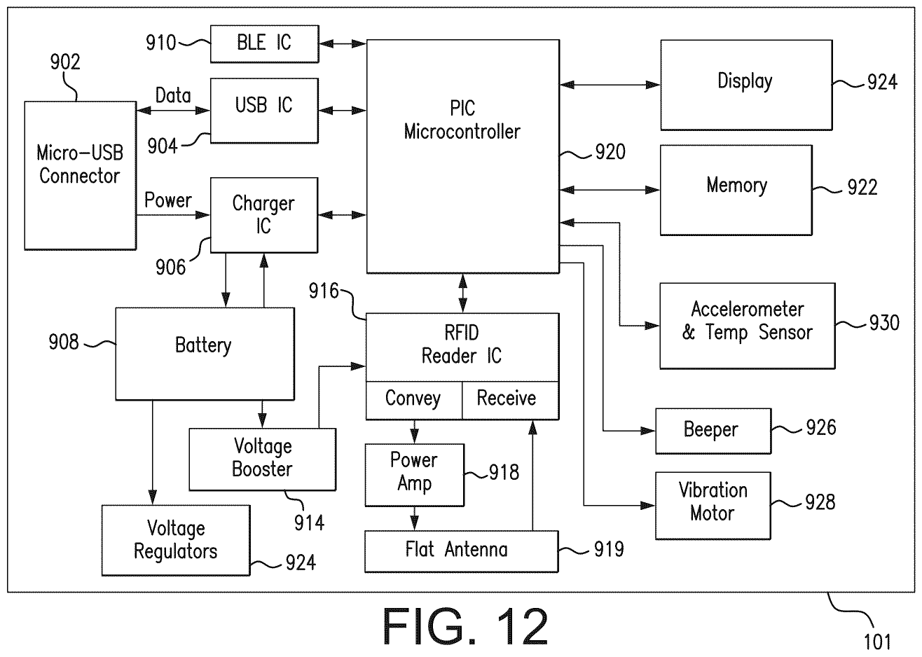

FIG. 12 is a schematic block diagram of an external transceiver 101 according to some aspects. In some aspects, the transceiver 101 may have a connector 902, such as, for example, a Micro-Universal Serial Bus (USB) connector. The connector 902 may enable a wired connection to an external device, such as a personal computer (e.g., personal computer 109) or a display device 105 (e.g., a smartphone).

In some aspects, the transceiver 101 may exchange data to and from the external device through the connector 902 and/or may receive power through the connector 902. The transceiver 101 may include a connector integrated circuit (IC) 904, such as, for example, a USB-IC, which may control transmission and receipt of data through the connector 902. The transceiver 101 may also include a charger IC 906, which may receive power via the connector 902 and charge a battery 908 (e.g., lithium-polymer battery). In some aspects, the battery 908 may be rechargeable, may have a short recharge duration, and/or may have a small size.

In some aspects, the transceiver 101 may include one or more connectors in addition to (or as an alternative to) Micro-USB connector 904. For example, in one alternative aspect, the transceiver 101 may include a spring-based connector (e.g., Pogo pin connector) in addition to (or as an alternative to) Micro-USB connector 904, and the transceiver 101 may use a connection established via the spring-based connector for wired communication to a personal computer (e.g., personal computer 109) or a display device 105 (e.g., a smartphone) and/or to receive power, which may be used, for example, to charge the battery 908.

In some aspects, as shown in FIG. 12, the transceiver 101 may have a wireless communication IC 910, which enables wireless communication with an external device, such as, for example, one or more personal computers (e.g., personal computer 109) or one or more display devices 107 (e.g., a smartphone). In some aspects, the wireless communication IC 910 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some aspects, the wireless communication IC 910 may be configured to wirelessly transmit data at a frequency greater than 1 gigahertz (e.g., 2.4 or 5 GHz). In some aspects, the wireless communication IC 910 may include an antenna (e.g., a Bluetooth antenna). In some aspects, the antenna of the wireless communication IC 910 may be entirely contained within the housing (e.g., housing 206 and 220) of the transceiver 101. However, this is not required, and, in alternative aspects, all or a portion of the antenna of the wireless communication IC 910 may be external to the transceiver housing.

In some aspects, the transceiver 101 may include a display interface device, which may enable communication by the transceiver 101 with one or more display devices 107. In some aspects, the display interface device may include the antenna of the wireless communication IC 910 and/or the connector 902. In some non-limiting aspects, the display interface device may additionally include the wireless communication IC 910 and/or the connector IC 904.

In some aspects, as shown in FIG. 12, the transceiver 101 may include voltage regulators 912 and/or a voltage booster 914. The battery 908 may supply power (via voltage booster 914) to radio-frequency identification (RFID) reader IC 916, which uses the inductive element 103 to convey information (e.g., commands) to the sensor 101 and receive information (e.g., measurement information) from the sensor 100. In some aspects, the sensor 100 and transceiver 101 may communicate using near field communication (NFC) (e.g., at a frequency of 13.56 MHz). In the illustrated aspect, the inductive element 103 is a flat antenna. In some aspects, the antenna may be flexible. However, as noted above, the inductive element 103 of the transceiver 101 may be in any configuration that permits adequate field strength to be achieved when brought within adequate physical proximity to the antenna 114 of the sensor 100. In some aspects, the transceiver 101 may include a power amplifier 918 to amplify the signal to be conveyed by the inductive element 103 to the sensor 100.

In some aspects, the transceiver 101 may include a peripheral interface controller (PIC) controller 920 and memory 922 (e.g., Flash memory), which may be non-volatile and/or capable of being electronically erased and/or rewritten. The PIC controller 920 may control the overall operation of the transceiver 101. For example, the PIC controller 920 may control the connector IC 904 or wireless communication IC 910 to transmit data via wired or wireless communication and/or control the RFID reader IC 916 to convey data via the inductive element 103. The PIC controller 920 may also control processing of data received via the inductive element 103, connector 902, or wireless communication IC 910.

In some aspects, the transceiver 101 may include a sensor interface device, which may enable communication by the transceiver 101 with a sensor 100. In some aspects, the sensor interface device may include the inductive element 103. In some non-limiting aspects, the sensor interface device may additionally include the RFID reader IC 916 and/or the power amplifier 918. However, in some alternative aspects where there exists a wired connection between the sensor 100 and the transceiver 101 (e.g., transcutaneous aspects), the sensor interface device may include the wired connection.

In some aspects, the transceiver 101 may include a display 924 (e.g., liquid crystal display and/or one or more light emitting diodes), which PIC controller 920 may control to display data (e.g., analyte concentration values). In some aspects, the transceiver 101 may include a speaker 926 (e.g., a beeper) and/or vibration motor 928, which may be activated, for example, in the event that an alarm condition (e.g., detection of a hypoglycemic or hyperglycemic condition) is met. The transceiver 101 may also include one or more additional sensors 930, which may include an accelerometer and/or temperature sensor that may be used in the processing performed by the PIC controller 920.

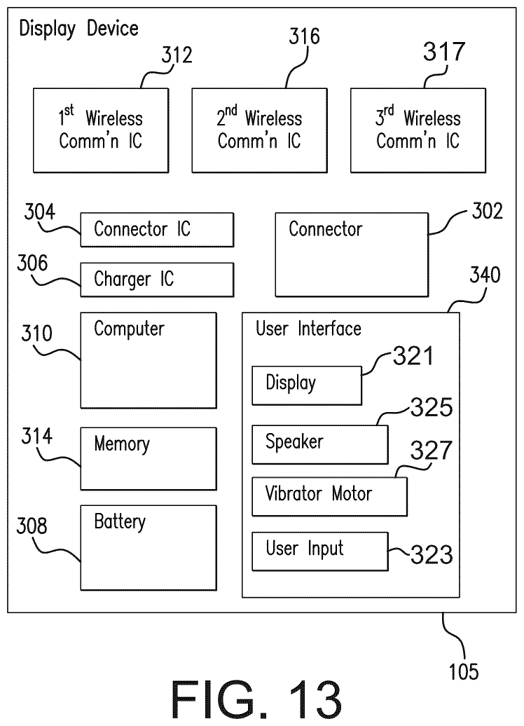

FIG. 13 is a block diagram of the display device 105 of the analyte monitoring system 50 according to some aspects. As shown in FIG. 13, in some aspects, the display device 105 may include one or more of a connector 302, a connector integrated circuit (IC) 304, a charger IC 306, a battery 308, a computer 310, a first wireless communication IC 312, a memory 314, a second wireless communication IC 316, a third wireless communication IC 317, and a user interface 340.

In some aspects in which the display device 105 includes the connector 302, the connector 302 may be, for example and without limitation, a Micro-Universal Serial Bus (USB) connector. The connector 302 may enable a wired connection to an external device, such as a personal computer or transceiver 101. The display device 105 may exchange data to and from the external device through the connector 302 and/or may receive power through the connector 302. In some aspects, the connector IC 304 may be, for example and without limitation, a USB-IC, which may control transmission and receipt of data through the connector 302.

In some aspects in which the display device 105 includes the charger IC 306, the charger IC 306 may receive power via the connector 302 and charge the battery 308. In some aspects, the battery 308 may be, for example and without limitation, a lithium-polymer battery. In some aspects, the battery 308 may be rechargeable, may have a short recharge duration, and/or may have a small size.

In some aspects, the display device 105 may include one or more connectors and/or one or more connector ICs in addition to (or as an alternative to) connector 302 and connector IC 304. For example, in some alternative aspects, the display device 105 may include a spring-based connector (e.g., Pogo pin connector) in addition to (or as an alternative to) connector 302, and the display device 105 may use a connection established via the spring-based connector for wired communication to a personal computer or the transceiver 101 and/or to receive power, which may be used, for example, to charge the battery 308.

In some aspects in which the display device 105 includes the first wireless communication IC 312, the first wireless communication IC 312 may enable wireless communication with one or more external devices, such as, for example, one or more personal computers, one or more transceivers 101, and/or one or more other display devices 105. In some aspects, the first wireless communication IC 312 may employ one or more wireless communication standards to wirelessly transmit data. The wireless communication standard employed may be any suitable wireless communication standard, such as an ANT standard, a Bluetooth standard, or a Bluetooth Low Energy (BLE) standard (e.g., BLE 4.0). In some aspects, the first wireless communication IC 312 may be configured to wirelessly transmit data at a frequency greater than 1 gigahertz (e.g., 2.4 or 5 GHz). In some aspects, the first wireless communication IC 312 may include an antenna (e.g., a Bluetooth antenna). In some aspects, the antenna of the first wireless communication IC 312 may be entirely contained within a housing of the display device 105. However, this is not required, and, in alternative aspects, all or a portion of the antenna of the first wireless communication IC 312 may be external to the display device housing.

In some aspects, the display device 105 may include a transceiver interface device, which may enable communication by the display device 105 with one or more transceivers 101. In some aspects, the transceiver interface device may include the antenna of the first wireless communication IC 312 and/or the connector 302. In some aspects, the transceiver interface device may additionally or alternatively include the first wireless communication IC 312 and/or the connector IC 304.

In some aspects in which the display device 105 includes the second wireless communication IC 316, the second wireless communication IC 316 may enable the display device 105 to communicate with one or more remote devices (e.g., smartphones, servers, and/or personal computers) via wireless local area networks (e.g., Wi-Fi), cellular networks, and/or the Internet. In some aspects, the second wireless communication IC 316 may employ one or more wireless communication standards to wirelessly transmit data. In some aspects, the second wireless communication IC 316 may include one or more antennas (e.g., a Wi-Fi antenna and/or one or more cellular antennas). In some aspects, the one or more antennas of the second wireless communication IC 316 may be entirely contained within a housing of the display device 105. However, this is not required, and, in alternative aspects, all or a portion of the one or more antennas of the second wireless communication IC 316 may be external to the display device housing.

In some aspects, the display device 105 may include a sensor interface device. In some aspects, the sensor interface device of the display device 105 may include the third wireless communication IC 317, and the third wireless communication IC 317 may enable the display device 105 to communicate directly with the sensor 100 so that the display device 105 may additionally perform some or all of the functions of the transceiver 101. In some aspects, the display device 105 and the sensor 100 may communicate using NFC (e.g. at a frequency of 13.56 MHz). In some aspects, the sensor interface device of the display device 105 may include an inductor (e.g. flat antenna, loop antenna, etc.) that is configured to permit adequate field strength to be achieved when brought within adequate physical proximity to the inductor 114 of the sensor 100. In some aspects, the display device 105 may receive sensor data from the sensor 100 periodically (e.g., every 1, 2, 5, 10, 15, or 20 minutes). In some aspects, the display device 105 may receive sensor data from the sensor 100 on demand (e.g., when the display device 100 is hovered or swiped in proximity to the sensor 100). In some aspects, the sensor interface device may additionally or alternatively include the RFID reader IC 916 and/or the power amplifier 918 described above with reference to FIG. 12. However, in some alternative aspects where there exists a wired connection between the sensor 100 and the display device 105 (e.g., transcutaneous aspects), the sensor interface device may include a wired connection to the analyte sensor 100.

In some aspects in which the display device 105 includes the memory 314, the memory 314 may be non-volatile and/or capable of being electronically erased and/or rewritten. In some aspects, the memory 314 may be, for example and without limitations a Flash memory.

In some aspects in which the display device 105 includes the computer 310, the computer 310 may control the overall operation of the display device 105. For example, the computer 310 may control the connector IC 304, the first wireless communication IC 312, the second wireless communication IC 316, and/or the third wireless communication IC 317 to transmit data via wired or wireless communication. The computer 310 may additionally or alternatively control processing of received data (e.g., analyte monitoring data received from the transceiver 101).

In some aspects in which the display device 105 includes the user interface 340, the user interface 340 may include a display 321 and/or a user input 323. In some aspects, the display 321 may be a liquid crystal display (LCD) and/or light emitting diode (LED) display. In some aspects, the user input 323 may include one or more buttons, a keyboard, a keypad, and/or a touchscreen. In some aspects, the computer 310 may control the display 321 to display data (e.g., analyte concentration values, analyte trend information, alerts, alarms, and/or notifications). In some aspects, the user interface 340 may include one or more of a speaker 325 (e.g., a beeper) and a vibration motor 327, which may be activated, for example, in the event that a condition (e.g., a hypoglycemic or hyperglycemic condition) is met.

In some aspects, the computer 310 may execute a mobile medical application (MMA). In some aspects, the display device 105 may receive analyte monitoring data from the transceiver 101. The received analyte monitoring data may include one or more analyte concentrations, one or more analyte concentrations trends, and/or one or more sensor measurements. The received analyte monitoring data may additionally or alternatively include alarms, alerts, and/or notifications. In some aspects, the display device 105 may receive measured analyte data directly from the sensor 100. The display device 105 may calculate an analyte concentration and an analyte concentration trend using at least the received sensor data. From this analyte information, the display device 105 may also determine if an alert and/or alarm condition exists, which may be signaled to the user (e.g., through vibration by a vibration motor and/or a display of a display device 105). In some aspects, this analyte information (e.g., calculated analyte concentrations, calculated analyte concentration trends, alerts, alarms, and/or notifications) may be displayed by the MMA being executed by the display device 105. In some aspects, the display device 105 may transmit this information (e.g., calculated analyte concentrations, calculated analyte concentration trends, alerts, alarms, and/or notifications) over a network such that a remote computing device (e.g., server) and one or more secondary display devices may receive, store, and display the analyte information.

In some aspects, the analyte monitoring system 50 may calibrate the conversion of raw sensor measurements to analyte concentrations. In some aspects, the calibration may be performed using one or more reference measurements (e.g., one or more self-monitoring blood glucose (SMBG) measurements). In some aspects, the reference measurements may be entered into the analyte monitoring system 50 using the user interface 340 of the display device 105. In some aspects, the display device 105 may convey one or more references measurements to the transceiver 101, and the transceiver 101 may use the one or more received reference measurements to perform the calibration. In some aspects, the display device 105 may additionally or alternatively use the one or more reference measurements to perform a calibration.

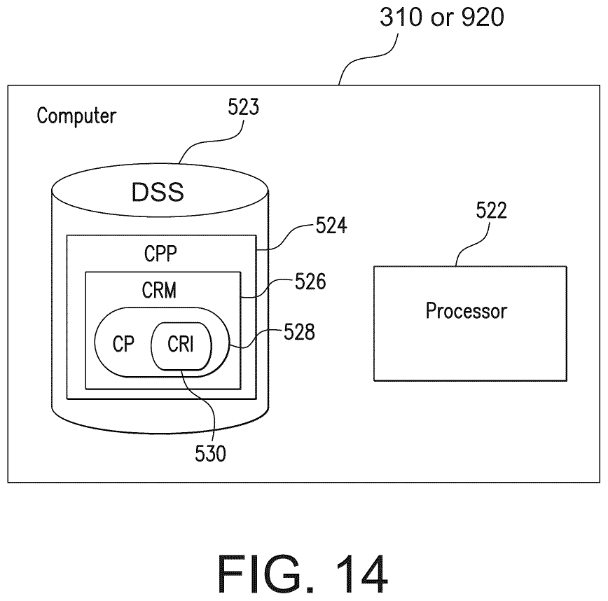

FIG. 14 is a block diagram of an aspect of a computer (e.g., the PIC controller 920 of the transceiver 101, the computer 310 of the display device 105, and/or a computer of the DMS 121) of the analyte monitoring system 50. As shown in FIG. 14, in some aspects, the computer may include processing circuitry 522 and/or one or more circuits, such as an application specific integrated circuit (ASIC), field-programmable gate arrays (FPGAs), a logic circuit, and the like. The processing circuitry 522 may include one or more processors (e.g., one or more general purpose microprocessors). In some aspects, the computer may include a data storage system (DSS) 523. The DSS 523 may include one or more non-volatile storage devices and/or one or more volatile storage devices (e.g., random access memory (RAM)). In aspects where the computer includes a processing circuitry 522, the DSS 523 may include a computer program product (CPP) 524. CPP 524 may include or be a computer readable medium (CRM) 526. The CRM 526 may store a computer program (CP) 528 comprising computer readable instructions (CRI) 530. In some aspects in which the computer is the computer 310 of the display device 105, the CRM 526 may store, among other programs, the MMA, and the CRI 530 may include one or more instructions of the MMA. The CRM 526 may be a non-transitory computer readable medium, such as, but not limited, to magnetic media (e.g., a hard disk), optical media (e.g., a DVD), solid state devices (e.g., random access memory (RAM) or flash memory), and the like. In some aspects, the CRI 530 of computer program 528 may be configured such that when executed by processing circuitry 522, the CRI 530 causes the computer to perform steps described below (e.g., steps described below with reference to the transceiver 101, display device 105, or DMS 121). In other aspects, the computer may be configured to perform steps described herein without the need for a computer program. That is, for example, the computer may consist merely of one or more ASICs. Hence, the features of the aspects described herein may be implemented in hardware and/or software.

In some aspects in which the user interface 340 of the display device 105 includes the display 321, the MMA may cause the display device 105 to provide a series of graphical control elements or widgets in the user interface 340, such as a graphical user interface (GUI), shown on the display 321. The MMA may, for example without limitation, cause the display device 105 to display analyte related information in a GUI such as, but not limited to: one or more of analyte information, current analyte concentrations, past analyte concentrations, predicted analyte concentrations, user notifications, analyte status alerts and alarms, trend graphs, arrows, and user-entered events. In some aspects, the MMA may provide one or more graphical control elements that may allow a user to manipulate aspects of the one or more display screens. Although aspects of the MMA are illustrated and described in the context of glucose monitoring system aspects, this is not required, and, in some alternative aspects, the MMA may be employed in other types of analyte monitoring systems.

In some aspects where the display device 105 communicates with a transceiver 101, which in turn obtains sensor measurement data from the analyte sensor 100, the MMA may cause the display device 105 to receive and display one or more of glucose data, trends, graphs, alarms, and alerts from the transceiver 101. In some aspects where the display device 105 communicates directly with the sensor 100 to obtain sensor measurement data, the MMA may cause the display device 105 to receive and display one or more of glucose data, trends, graphs, alarms, and alerts from the transceiver 101. In some aspects, the MMA may store glucose level history and statistics for a patient on the display device 105 (e.g., in memory 314 and/or DSS 523) and/or in a remote data storage system.

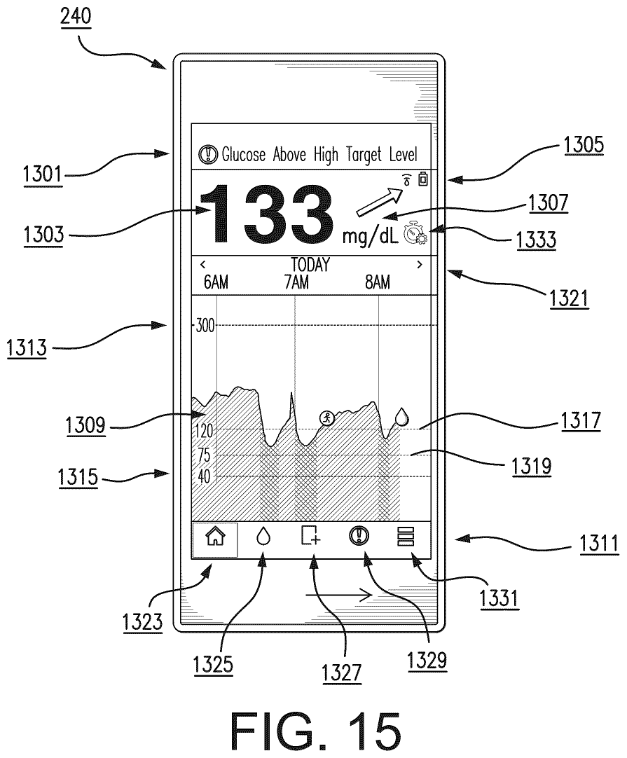

FIG. 15 is an example of a home screen display of a medical mobile application (MMA) in accordance with aspects of various aspects of the present invention. According to some aspects, the workspace display of the MMA may be depicted in a GUI on the display 321 of the display device 105. In some aspects, the home screen may display one or more of real-time analyte concentrations either received from transceiver 101 or calculated by the display device 105, rate and direction of analyte level change, graphical trends of analyte levels, alarms or alerts for hypoglycemia or hyperglycemia, and logged events such as, for example and without limitation, meals, exercise, and medications. Table 1 below depicts several informational non-limiting examples of items and features that may be depicted on the home screen.

TABLE 1
Home Screen

| | |
|---|---|
| Status bar | Shows the status of user's glucose level |
| Transceiver/ | This is the transceiver being used; the |
| Transmitter ID | transceiver name can be changed by going to |
| | Settings > System |
| Current glucose value | A real-time glucose reading; this may be |
| | updated every 5 minutes |
| Date and time | The current date and time with navigational |
| | options, such as scroll left or right to see |
| | different dates and times |
| Alarm and Events | Shows an icon when an alert, alarm, or event |
| | occurs |
| Bluetooth Connection | Shows the strength of the Bluetooth connection |
| Handheld Device Battery | Indicates the battery strength of the handheld |
| Level | device |
| Transmitter/Transceiver | Indicates the battery strength of the transceiver |
| Battery Level | |
| Transmitter/Transceiver | Shows the strength of the transceiver |
| Connection Status Icon | connection |
| Trend Arrow | Shows the direction a patient's glucose level is |
| | trending [symbol] |
| Unit of Measurement | This is the units for the glucose value |
| High Glucose Alarm | This is the high glucose alarm or alert level set |
| Level | by a use [symbol] |
| Glucose High Target | This is the high glucose target level set by a |
| Level | user |
| Stacked Alerts | Shows when there are several alerts at the same |
| | time |
| Glucose Trend Graph | A user can navigate or scroll through the graph |
| | to see the trend over time |
| Menu | Navigation to various sections of the MMA, |
| | such as: |
| | Home Reports |
| | Settings |
| | Calibrate Share My Data Abo [symbol] |
| | Notifications Placement Guide |
| | Event Log Connect |
| Calibration Point Icon | This icon appears when a calibration is entered |
| Profile Indicator | This indicator may indicate what profile is |
| | being applie [symbol] such as a normal profile, |
| | temporary profile, vacation profil [symbol] and the |
| | like. |

[symbol]

indicates data missing or illegible when filed

In some aspects, as shown in FIG. 15, the home screen may include one or more of a status notification bar 1301, a real-time current glucose level 1303 of a patient, one or more icons 1305, a trend arrow 1307, a historical graph 1309, a profile indicator 1333, and navigation tools 1311. The status notification bar 1301 may depict, for example and without limitation, alarms, alerts, and notifications related to, for example, glucose levels and system statistics and/or status. The one or more icons 1305 may represent the signal strength of the transceiver 101 and/or the battery level of the transceiver 101. The trend arrow 1307 may indicate a rate and/or direction of change in glucose measurements of a patient. The historical graph may be, for example and without limitation, a line graph and may indicate trends of glucose measurement levels of a patient. The navigation tools 1311 may allow a user to navigate through different areas or screens of the MMA. The screens may include, for example and without limitation, one or more of Home, Calibrate, Event Log, Notifications, and Menu screens.

In some aspects, the historical graph 1309 may depict logged events and/or user inputted activities such as meals (nutrition, amount of carbohydrates), exercise (amount of exercise), medication (amount of insulin units), and blood glucose values as icons on positions of the graph corresponding to when such events occurred. In some aspects, the historical graph 1309 may show one or more of a boundary or indication of a high glucose alarm level 1313, a low glucose alarm level 1315, a high glucose target level 1317, and a low glucose target level 1319. In some aspects, a user may interact with a time or date range 1321 option via the GUI to adjust the time period of the glucose level displayed on the historical graph 1309. In some aspects, the date range 1321 may be specified by a user and may bet set to different time periods such as 1, 3, 24 hours, 1, 7, 14, 30, and 60 days, weeks, months, etc. In some aspects, the line graph 1309 may show high, low, and average glucose levels of a patient for the selected date range 1321. In other aspects, the line graph 1309 may be a pie chart, log book, modal day, or other depiction of glucose levels of a patient over a selectable date range 1321, any of which may further depict high, low, and average glucose levels of the patient over that date range 1321.

In some aspects, the trend arrow 1307 may be depicted in five different configurations that signify direction (up, down, neutral) and rate (rapidly, very rapidly slow, slow, very slow, and stable) of glucose change. In some aspects, the MMA and/or the transceiver 101 may use the last twenty minutes of glucose measurement data received from the sensor 101 and/or processed by the transceiver 730 in the calculation used to determine the orientation of the trend arrow 1307. In some aspects, there may be times when the trend arrow 1307 may not be displayed due to, for example, there being insufficient sensor values available for the trend calculation. In some aspects, a trend arrow 1307 displayed in a horizontal orientation (approximately 0° along the horizontal direction of the GUI display) may indicate that the glucose level is changing gradually, such as, for example, at a rate between −1.0 mg/dL and 1.0 mg/dL per minute. In some aspects, a trend arrow 1307 displayed slightly in the upwards direction (approximately 45° up from the horizontal direction of the GUI display) may indicate that the glucose level is rising moderately, such as, for example, at a rate between 1.0 mg/dL and 2.0 mg/dL per minute. In some aspects, a trend arrow 1307 displayed slightly in the downwards direction (approximately 45° down from the horizontal direction of the GUI display) may indicate that the glucose level is falling moderately, such as, for example, at a rate between 1.0 mg/dL and 2.0 mg/dL per minute. In some aspects, a trend arrow 1307 displayed in a vertical direction (approximately 90° up from the horizontal direction of the GUI display) may indicate that the glucose level is rising very rapidly, such as, for example, at a rate more than 2.0 mg/dL per minute. In some aspects, a trend arrow 1307 displayed in a downwards direction (approximately 90° down from the horizontal direction of the GUI display) may indicate that the glucose level is falling very rapidly, such as, for example, at a rate more than 2.0 mg/dL per minute. In some aspects, the trend arrow 1307 is different from a predicted glucose alarm or alert. For example, the trend arrow 1307 may indicate rate and direction of change regardless of glucose value, whereas predicted glucose alarms or alerts may indicate reaching a certain glucose level based on current trends. For example, the MMA may cause a predicted low glucose alarm or alert to be displayed in the notification bar 1301 while still displaying a relatively stable trend arrow 1307 (e.g., at 0° or 45° from the horizontal direction of the GUI display).

In some aspects, the historical line graph 1309 may allow user to quickly review and analyze historical data and/or trend information of a patient's glucose levels over time. In some aspects, the historical line graph 1309 may include icons or markers along the trend line to reflect alarms, alerts, notifications, and/or any events that were automatically or manually logged by the user into the display device 105 via a GUI display generated by the MMA. Where one or more of such icons or markers are displayed on the historical line graph 1309, a user may select any one of the icons or markers to obtain more information about the item. For example, in response to a selection of a mark on the line graph 1309, the MMA may generate a popup window on the display 220 that provides more information about the mark.

In some aspects, the historical line graph 1309 may enable a user to quickly review how well a patient is doing against glucose targets and/or alarms or alerts. For example, a user may establish a high glucose alarm level 1313 and/or a low glucose alarm level 1315, as well as a high glucose target level 1317 and/or a low glucose target level 1319. The high glucose alarm level 1313 and/or low glucose alarm level 1315 may be visually depicted over the historical line graph 1309, for example, using a colored dashed line (such as red). Additionally, the high glucose target level 1317 and low glucose target level 1319 may be visually depicted over the historical line graph 1309, for example, using a color dashed line (such as green).

In some aspects, the colors of the historical line graph 1309 may change depending on a glucose level 1303 status. For example, during the times where the glucose level 1303 was outside of the high glucose alarm level 1313 or low glucose alarm level 1315, then the portion of the line graph 1309 corresponding to those times may be filled in red. As another example, during the times where the glucose level 1303 is between the high glucose target level 1317 and the low glucose target level 1319, then the portion of the line graph 1309 corresponding to those times may be filled in green. As yet another example, during the times where the glucose level 1303 is between a glucose target level 1317, 1319 and a corresponding alarm level 1313, 1315, then the portion of the line graph 1309 may be filled in yellow.

In some aspects, the line graph 1309 may be displayed with one or more selectable date range icons 1321 that allow a user to change the day/time period corresponding to the line graph 1309 in real-time. For example, a user may select a forwards or backwards selectable option (such as an arrow) or use a swipe or fling gesture that may be recognized by GUI to navigate to a later or earlier time period, respectively, such as a day, month, etc. In some aspects a user may choose an older graph 1309 to display by tapping the date on the date range 1321 portion of the screen and submitting or entering a desired date and/or time to review. In some aspects, a user may use one or more gestures that are recognized by the GUI, such as a pinch, zoom, tap, press and hold, or swipe, on graph 1309. For example, a user may pinch the historical line graph 1309 with a thumb and index finger in order to cause the MMA to display different time/dating settings or adjust a time/date setting on the line graph 1309. In some aspects, a user may tap or press and hold a time event on historical line graph 1309, and in response the MMA may display further detail on the time event, such as a history, reading value, date/time, or association to other events or display a prompt for entry of a time event.

In some aspects, the MMA may store glucose data 1303 on the display device 105 (e.g., in memory 314 and/or DSS 523) so long as there is available memory space. Additionally or alternatively, the MMA may cause the display device 105 to send a sync request message to store the glucose data 1303 on a remote storage device.

In some aspects, the MMA may cause the GUI to display navigational tools 1311 that allow a user to navigate to different features and screens provided by the MMA. For example, the navigational tools 1311 may include a navigation bar with one or more of a plurality of selectable navigation options 1323, 1325, 1327, 1329, and 1331, such as buttons or icons. In some aspects, the selectable navigation options may allow a user to navigate to one or more of the “Home” screen 1323, a “Calibrate” screen 1325, an “Event Log” screen 1327, a “Notifications” screen 1329, and a “Menu” screen 1331. Upon a user selection of one of the selectable navigation options in the navigation tools area 1311, a new screen corresponding to the selected option may be displayed on a display device by the GU

In some aspects where the system includes the data management system (DMS) 121 (see FIG. 1), the DMS 121 may be a web-based analyte DMS. In some aspects, the DMS 121 may be a server device employed to allow data to be shared over the network such as the Internet. In some aspects, data from the display device 105 and/or PC 109 may be uploaded (e.g., through a wired connection such as, for example, a USB connection or a wireless connection such as, for example, a wireless Internet connection) to a web server on a remote computer. In some aspects, the DMS 121 may enable sharing of the analyte data (e.g., allowing the user, caregiver, and/or clinician to view sensor analyte data). The user may collect analyte data at home or in a clinic/research facility and then upload the data to their computer web account. Using the web account, the DMS 121 may use the data to generate one or more different reports utilizing the uploaded information. For example, in some aspects, the DMS 121 may use the uploaded data to generate one or more of the following reports: (i) an analyte details report, (ii) an analyte line report, (iii) a modal day report, (iv) a modal summary report, (v) a statistics report, and (vi) a transceiver log report.

In some aspects, a user may use the DMS 121 to register with the DMS 121 and create a unique user ID and password. Once logged in, the user may enter their basic user information and may upload analyte reading data from their transceiver 101 or display device 105. In various aspects, the DMS 121 may support specific data types such as, for example, glucose, insulin, meal/carbs, exercise, health event, alarms, and errors. In some non-limiting aspects, data can be automatically uploaded or entered manually by the user or imported from the transceiver 101 and then saved in the DMS 121 to be viewed at a later date.

In some aspects, the analyte monitoring system 50 may be used as a continuous analyte monitoring system (e.g., continuous glucose monitoring (CGM) system). In some aspects, the analyte monitoring system 50 may additionally or alternatively be used as a flash analyte monitoring system (e.g., a flash glucose monitoring (FGM) system). For example, a user may use the analyte monitoring system 50 as a flash analyte monitoring system during day (and not wear the transceiver 101) and as a continuous analyte monitoring system at night (e.g., such that the transceiver 101 would provide on-body vibration alerts if the user's analyte concentration gets too high or too low while the user is sleeping).

In some aspects, using the analyte monitoring system 50 as a continuous analyte monitoring system may include a user wearing the transceiver 101 and receiving (e.g., read) sensor measurements from the analyte sensor 101 at regular intervals (e.g., every 5 minutes). In some aspects, the analyte sensor 100 may be powered by a charge storage device 202 (e.g., a battery), and the sensor measurements received by the transceiver 101 may be autonomous sensor measurements. In some alternative aspects, the analyte sensor 100 may be powered by the transceiver 101 (and may not include a battery), and the sensor measurements received by the transceiver 101 may have been requested by the transceiver 101 (e.g., by conveying a measurement command to the analyte sensor 100). In some aspects, using the analyte monitoring system 50 as a continuous analyte monitoring system may include the transceiver 101 providing on-body (e.g., vibration) alerts to the user. In some aspects, using the analyte monitoring system 50 as a continuous analyte monitoring system may include the transceiver 101 using the sensor measurements (e.g., one or more analyte measurements, one or more interferent measurements, one or more reference measurements, one or more temperature measurements, and/or one or more voltage measurements) to calculate analyte concentrations and conveying the calculated analyte concentrations to the display device 105 (e.g., mobile device or smartphone) for display by a mobile medical application (MMA) being executed by the display device 105.

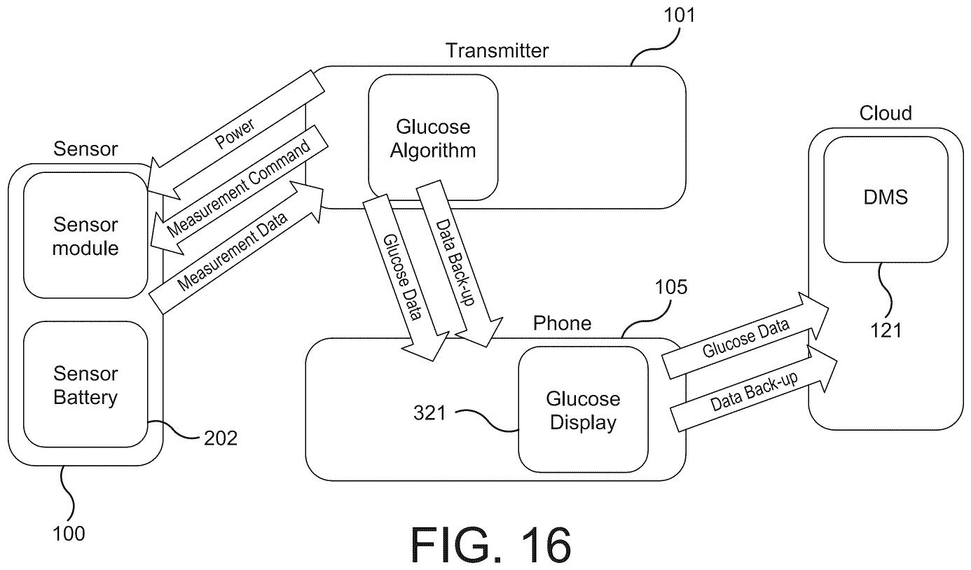

For example, as shown in FIG. 16, in some aspects in which the analyte monitoring system 50 is used as a continuous analyte monitoring system, the transceiver 101 may power the analyte sensor 10, which may or may not have a charge storage device 202. In some aspects, as shown in FIG. 16, the transceiver 101 may request sensor measurements by conveying a measurement command to the analyte sensor 100. In some aspects, the analyte sensor 100 may receive the measurement command and, in response, take sensor measurements (e.g., one or more analyte measurements, one or more interferent measurements, one or more reference measurements, one or more temperature measurements, and/or one or more voltage measurements) using power received from the transceiver 101. In some aspects, as shown in FIG. 16, the analyte sensor 100 may convey the measurement data to the transceiver 101.

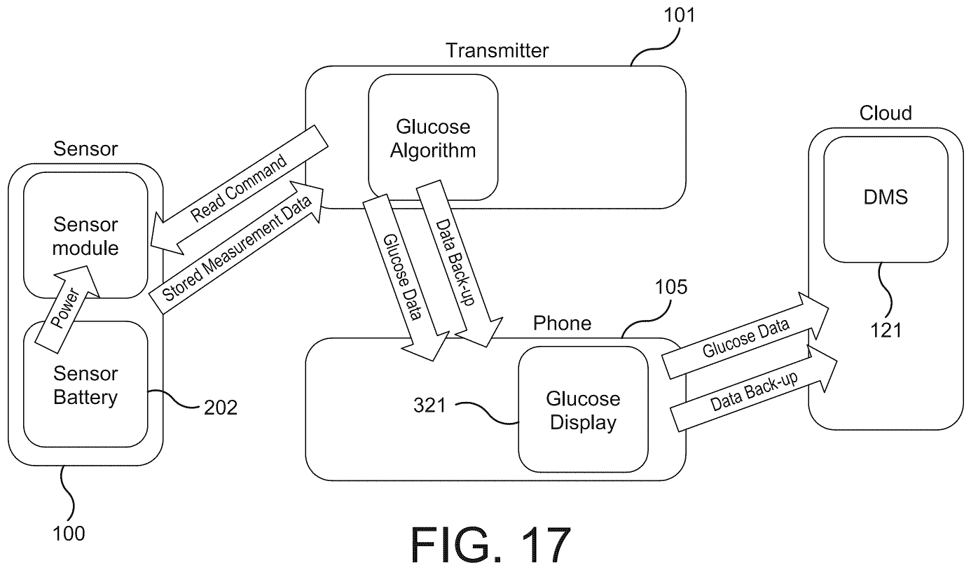

For another example, as shown in FIG. 17, in some alternative aspects in which the analyte monitoring system 50 is used as a continuous analyte monitoring system, the analyte sensor 100 may be powered by a charge storage device 202 (e.g., a battery), and the analyte sensor 100 may take autonomous sensor measurements (e.g., at regular intervals such as, for example, every 5 minutes) and store the autonomous sensor measurements in a memory (e.g., memory 824). In some aspects, the transceiver 101 may request stored sensor measurements (e.g., by conveying one or more read requests/commands to the analyte sensor 100), and the analyte sensor 100 may convey the autonomous sensor measurements to the transceiver 101 in response to receiving the one or more read requests from the transceiver 101.

In some aspects in which the analyte monitoring system 50 is used as a continuous analyte monitoring system, as shown in FIGS. 16 and 17, the transceiver 101 may use the sensor measurements to calculate an analyte concentration and convey the calculated analyte concentration (e.g., glucose data) to the display device 105. In some aspects, the display device 105 may display the calculated analyte concentration (e.g., using the display 321 of the display device 105). In some aspects, display device 105 may convey the calculated analyte concentration to the DMS 121. In some alternative aspects, the transceiver 101 may not calculate analyte concentrations and may instead convey the sensor measurements to the display device 105, which uses the sensor measurements to calculate an analyte concentration. In some other alternative aspects, the transceiver 101 may not calculate analyte concentrations and may instead convey the sensor measurements to the display device 105, which conveys the sensor measurements to the DMS 121, which uses the sensor measurements to calculate an analyte concentration.

In some aspects, as shown in FIGS. 16 and 17, the transceiver 101 may convey back-up data (e.g., sensor measurements) to the display device 105, and the display device 105 may convey the back-up data to the DMS 121. In some aspects, the data back-up may occur in real-time. In some alternative aspects, the data back-up does not need to occur in real-time and may only occur when display device 105 is available (e.g., when the display device is within the communication range of the transceiver 101, and the transceiver 101 is able to convey back-up data to the display device 105). In some aspects, the back-up data may be used to restore data to a replacement transceiver 101 (e.g., if the original transceiver 101 is lost, stolen, or damaged).

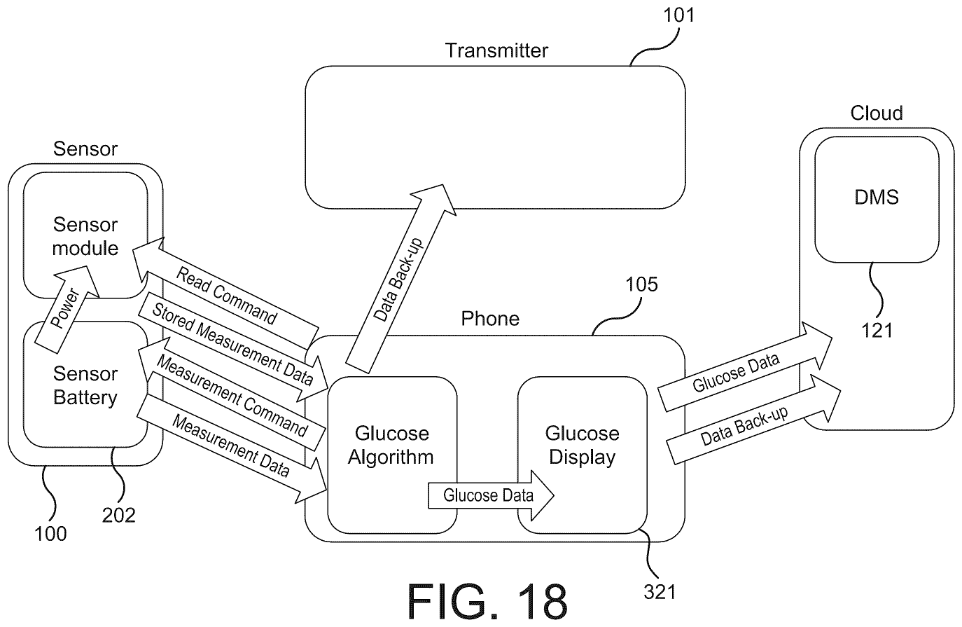

In some aspects, as shown in FIGS. 18-20, using the analyte monitoring system as a flash analyte monitoring system may include using the display device 105 to receive (e.g., read) sensor measurements directly from the analyte sensor 100 (e.g., using near field communication (NFC)). In some aspects, to receive sensor measurements directly from the analyte sensor 100, the user may place the display device 105 over the analyte sensor 100, and the display device 105 may readout a sensor measurements stored by the analyte sensor 100. In some aspects, as shown in FIGS. 18-20, the analyte sensor 100 may be powered by a charge storage device 202 (e.g., a battery), and the sensor measurements received by the display device 105 may be autonomous sensor measurements. In some aspects, while using the analyte monitoring system 50 as a flash analyte monitoring system, the user may not need to wear or use the transceiver 101.

In some aspects, as shown in FIG. 18, using the analyte monitoring system 50 as a flash analyte monitoring system may include the display device 105 using the sensor measurements (e.g., one or more analyte measurements, one or more interferent measurements, one or more reference measurements, one or more temperature measurements, and/or one or more voltage measurements) to calculate analyte concentrations. In some aspects, as shown in FIG. 18, the display device 105 may display the calculated analyte concentration (e.g., using the display 321 of the display device 105). In some aspects, as shown in FIG. 18, display device 105 may convey the calculated analyte concentration to the DMS 121.

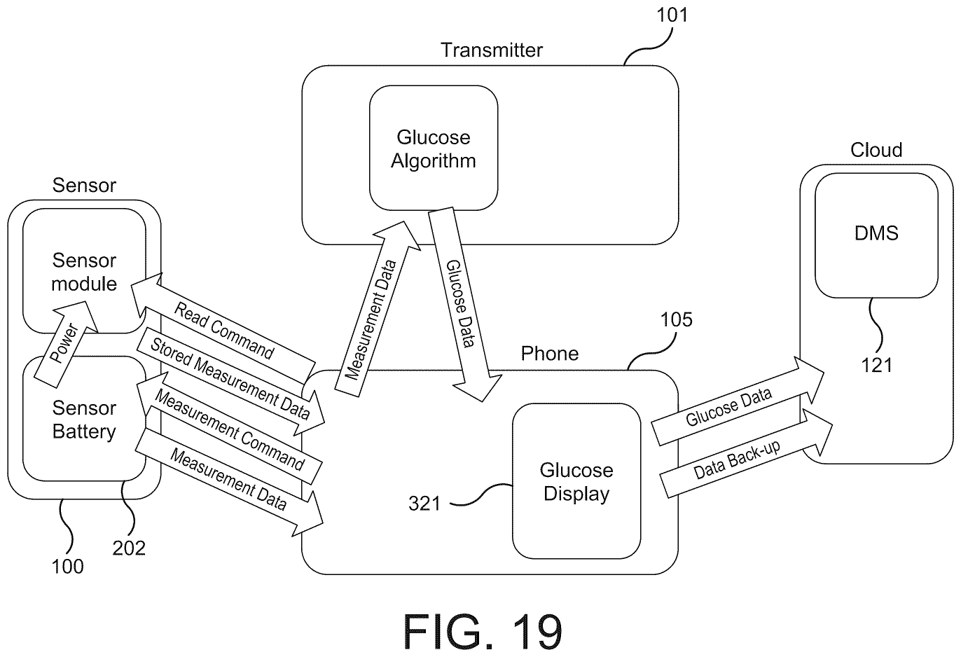

In some alternative aspects, as shown in FIG. 19, using the analyte monitoring system 50 as a flash analyte monitoring system may additionally or alternatively include the display device 105 conveying the sensor measurements to the transceiver 101, the transceiver 101 using the sensor measurements to calculate analyte concentrations, the transceiver 101 conveying the calculated analyte concentrations to the display device 105, and the display device 105 receiving the calculated analyte concentrations. In some aspects, using the transceiver 101 to calculate analyte concentrations may require the transceiver 101 to be present (e.g., so that the transceiver 101 can receive sensor measurements and convey calculated analyte concentrations to the display device 105) but does not require the transceiver 101 to be on the body of the user. In some aspects, as shown in FIG. 19, the display device 105 may display the calculated analyte concentration (e.g., using the display 321 of the display device 105). In some aspects, as shown in FIG. 19, display device 105 may convey the calculated analyte concentration to the DMS 121.

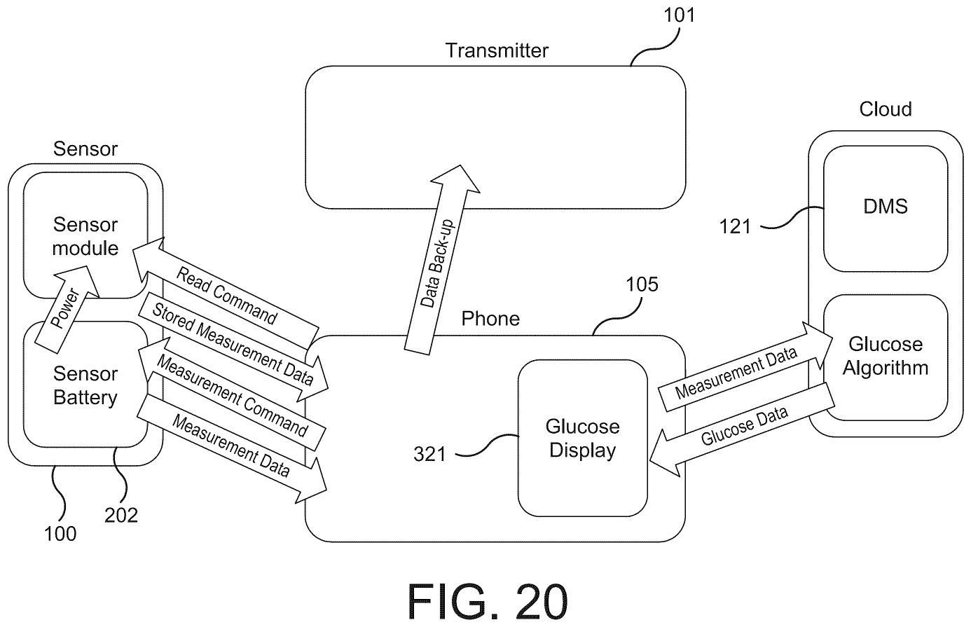

In some alternative aspects, as shown in FIG. 20, using the analyte monitoring system 50 as a flash analyte monitoring system may additionally or alternatively include the display device 105 conveying the sensor measurements to the DMS 121, the DMS 121 using the sensor measurements to calculate analyte concentrations, the DMS 121 conveying the calculated analyte concentrations to the display device 105, and the display device 105 receiving the calculated analyte concentrations. In some aspects, as shown in FIG. 20, the display device 105 may display the calculated analyte concentration (e.g., using the display 321 of the display device 105).

In some aspects, as shown in FIGS. 18-20, the display device 105 may convey back-up data (e.g., sensor measurements) to the transceiver 101 and/or the DMS 121. In some aspects, the data back-up may occur in real-time. In some alternative aspects, the data back-up does not need to occur in real-time and may occur only when the transceiver 101 and/or the DMS 121 is available (e.g., when display device 105 is able to convey back-up data to the transceiver 101 and/or the DMS 121). In some aspects, the back-up data may be used to restore data to a replacement display device 105 (e.g., if a display device 105 is lost, stolen, or damaged). In some aspects, if both the transceiver 101 and the display device 105 are lost, stolen, or damaged, back-up data conveyed to the DMS 121 may be used to restore data to a replacement display device 105, which may then restore data to a replacement transceiver 101.

In some aspects in which the analyte sensor 100 includes a charge storage device 202, each sensing device (e.g., each of the first and second sensing devices 100A and 100B) of the analyte sensor 100 may take autonomous sensor measurements and store them in a memory 824 of the sensing device, which may be fabricated in or mounted on a substrate 112 (e.g., first substrate 112a or second substrate 112b) of the sensing device. In some aspects, the sensing devices of the analyte sensor 100 may take the autonomous sensor measurements at regular intervals of time (e.g., at 30 second, 1 minute, 3 minute, 5 minute, 10 minute, or 15 minute intervals of time). That is, the sensing devices of the analyte sensor 100 may take the autonomous sensor measurements at a measurement frequency (e.g., every 30 seconds, every 1 minute, every 3 minutes, every 5 minutes, every 10 minutes, or every 15 minutes). In some aspects, although the analyte sensor 100 may take the autonomous sensor measurements at regular intervals, the exact time at which the measurement sequence is performed may be voltage and/or temperature dependent (e.g., changes in voltage and/or temperature may cause the length of the cycles of the clock 830, which may be used to determine the times at which measurement sequences are performed, to change). In some aspects, an autonomous sensor measurement sequence performed at one instance of time may produce a set of sensor measurements including one or more analyte measurements (e.g., indicative of the amount of first emission light 331 emitted by the analyte indicator 207 and received by one or more signal photodetectors 224), one or more interferent measurements (e.g., indicative of the amount of second emission light 332 emitted by the interferent indicator 209 and received by the one or more interferent photodetectors 228), one or more first reference measurements (e.g., indicative of the level of first excitation light 329 reflected from the indicator element 106 and received by the one or more reference photodetectors 226), one or more second reference measurements (e.g., indicative of the level of second excitation light 330 reflected from the indicator element 106 and received by the one or more signal photodetectors 224 or the one or more second reference photodetectors 230), one or more temperature measurements (e.g., generated by a temperature transducer 464 or 492 of the sensor elements 832), one or more field current measurement values (e.g., Icouple), one or more impedance measurements (e.g., one or more measurements of impedances of the light sources 108 and/or 227), one or more measurements of the voltage VBAT produced by the charge storage device 202, and/or timing information (e.g., a count of the cycles of the clock 830 and/or a number n for the autonomous sensor measurement). In some aspects, the set of autonomous sensor measurements taken at approximately one instance of time may include sensor measurements (e.g., one or more analyte measurements, one or more interferent measurements, one or more first reference measurements, one or more second reference measurements) from each sensor area 2202 of the sensing device (e.g., from each of sensing areas 2202a and 2202c of first sensing device 100A). In some aspects, the timing information may include a count of the cycles of the clock 830 (e.g., since autonomous measurements were started) at the instance of time. In some aspects, the timing information may additionally or alternatively include a number n for the autonomous sensor measurement (e.g., indicating that the autonomous sensor measurement is the nth autonomous sensor measurement taken since autonomous measurements were started). In some aspects, the set of autonomous sensor measurements from each sensor area 2202 of the sensing device may be stored in the memory 824 of the sensing area 2202.

In some aspects, the memories 824 of the sensing devices (e.g., sensing device 100A or 100B) of the analyte sensor 100 may have a finite capacity and, thus, may be able to store only a finite number of autonomous sensor measurements (e.g., with each autonomous sensor measurement including a set of sensor measurements). In some aspects, the memories 824 of the analyte sensor 100 may be capable of storing, for example and without limitation, 16, 20, 32, 40, 64, 100, 128, 200, or 256 autonomous sensor measurements. In some aspects, taking each autonomous sensor measurement may include a measurement cycle for each of the sensing areas (e.g., sensing areas 2202a-2202d). In some aspects including multiple sensing devices (e.g., first and second sensing devices 100A and 100B) that each include multiple sensing areas (e.g., first and second sensing devices 100A and 100B each including two sensing areas for a total of four sensing areas 2202a-2202d), for each autonomous sensor measurement, a memory 824 of each sensing device may store sensor measurements produced by a measurement cycle for each sensing area of the sensing device (e.g., for each autonomous sensor measurement, a memory 824 of the sensing device 100A may store sensor measurements produced by a measurement cycle for sensing area 2202a and sensor measurements produced by a measurement cycle for sensing area 2202c, and a memory 824 of the sensing device 100B may store sensor measurements produced by a measurement cycle for sensing area 2202b and sensor measurements produced by a measurement cycle for sensing area 2202d).

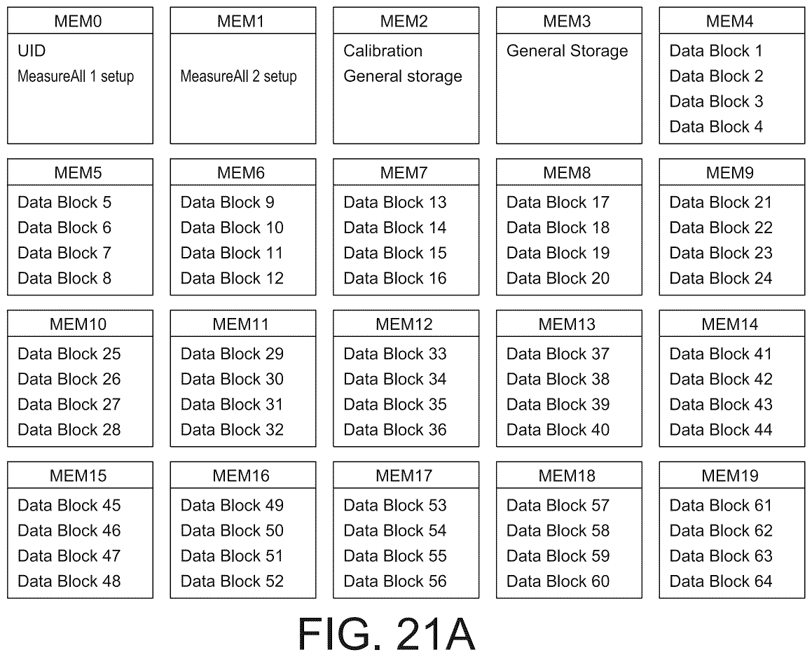

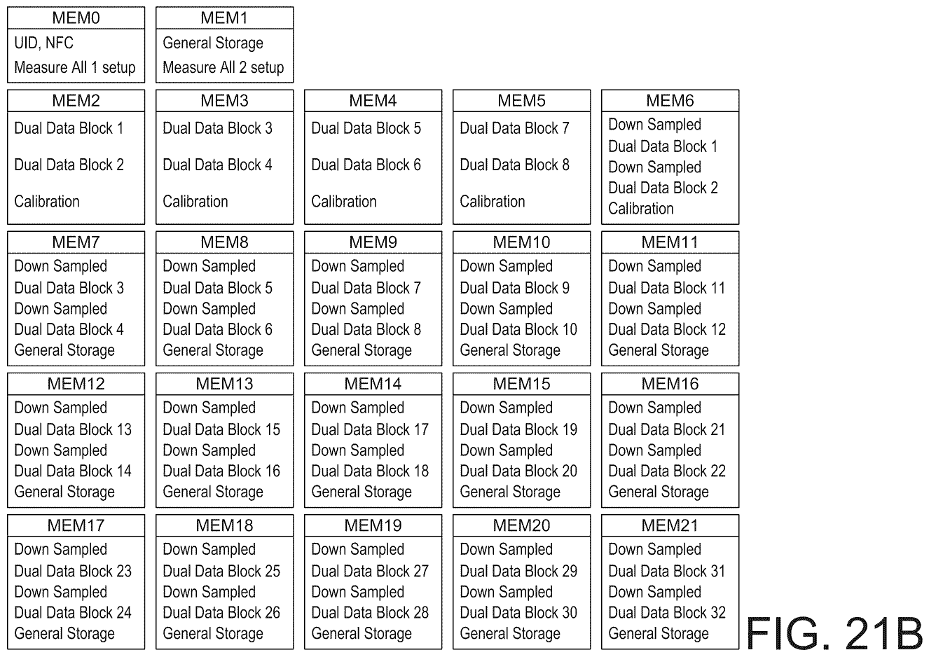

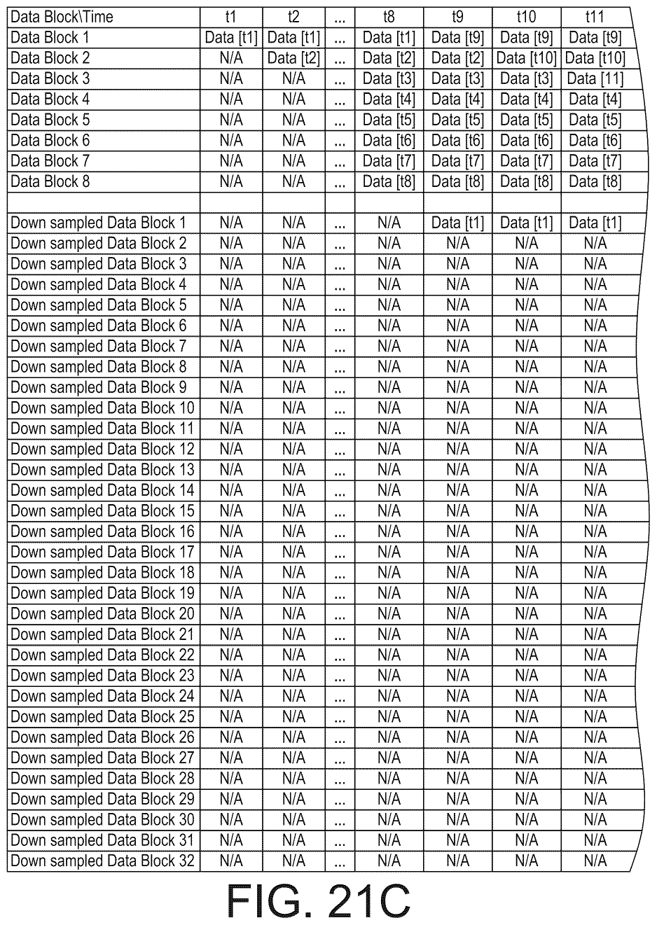

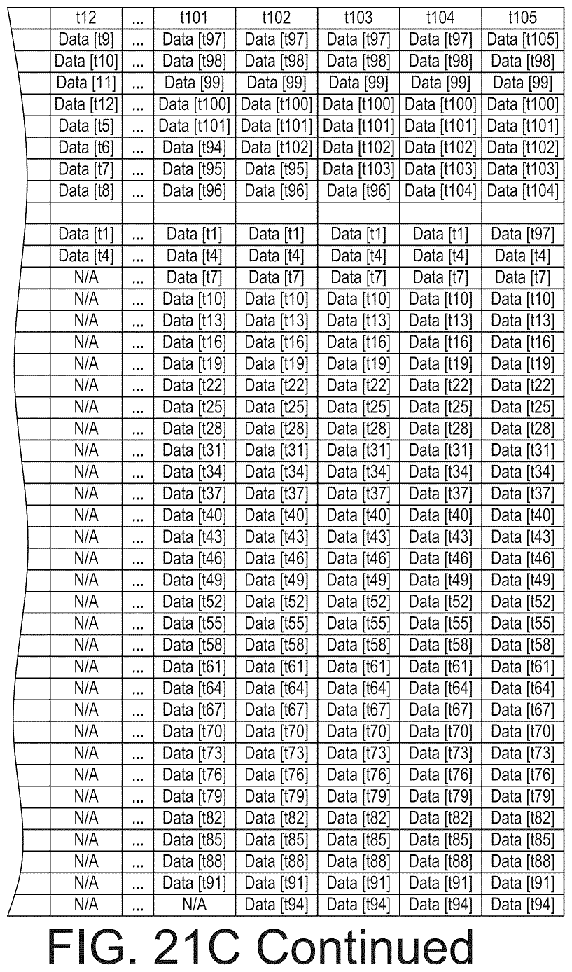

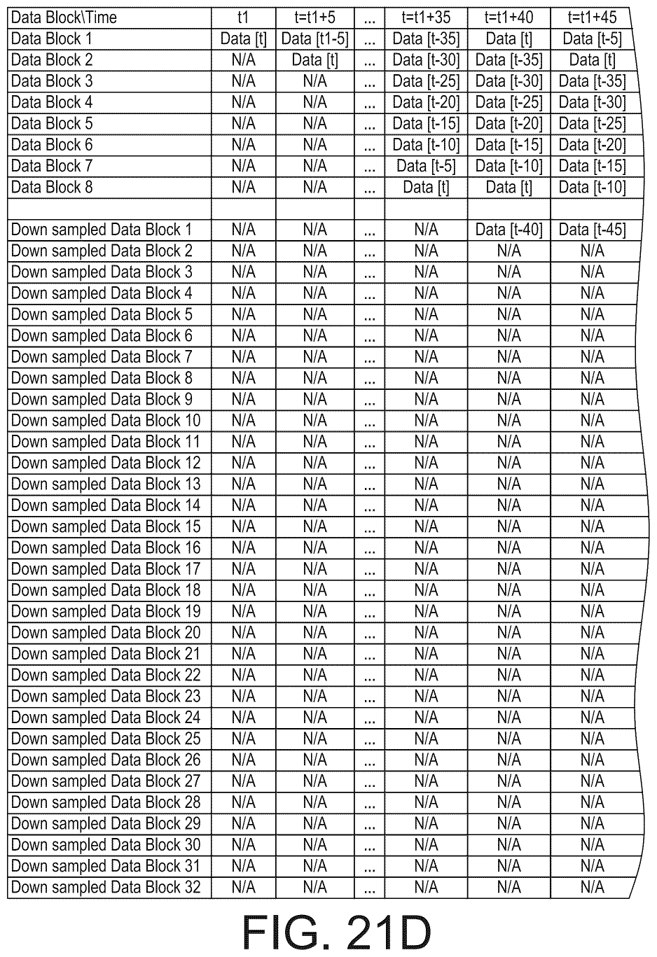

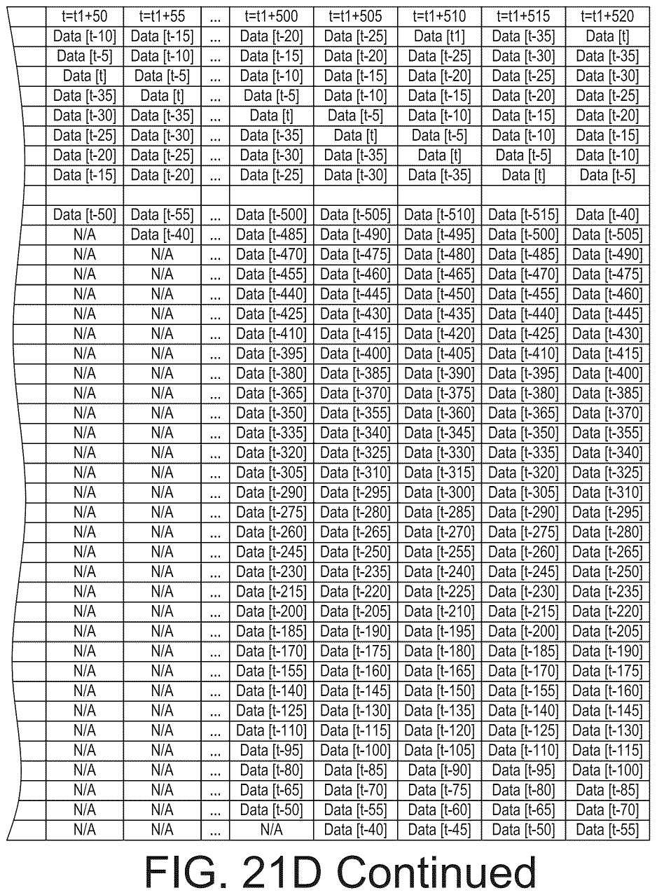

In some aspects in which the memories 824 of the analyte sensor 100 are capable of storing 32 autonomous sensor measurements, the memories 824 of the sensing devices of the analyte sensor 100 may each have the configuration shown in FIG. 21A. In some aspects, as shown in FIG. 21A, a memory 824 of a sensing device of the analyte sensor 100 may have 20 memory pages (i.e., MEM0 to MEM19). In some aspects, each of the memory pages may include, for example and without limitation, sixty-four 16-bit registers. In some aspects, some of the memory pages (e.g., MEM0 and MEM1) may store configuration information (e.g., an identification of the analyte sensor 100, an identification of the sensing device of the analyte sensor 100, and/or first and second measurement cycle setup parameters). In some aspects, the identifications of the analyte sensor 100 and of the sensing device may be unique identifiers (UIDs). In some aspects, the first and second measurements cycle setup parameters may be for first and second sensor areas, respectively, of the sensing device (e.g., sensing areas 2202a and 2202c, respectively, of the sensing device 100A or sensing areas 2202b and 2202d, respectively, of the sensing device 100B). In some aspects, the first and second measurement cycle setup parameters may include an automatic wait parameter indicative of whether the sensing device (e.g., sensing device 100a) of the analyte device 100 should delay for a period of time before performing an autonomous measurement sequence (e.g., to avoid interference with another sensing device, such as sensing device 100b, of the analyte sensor 100 that might occur if the sensing devices perform autonomous measurement sequences at the same time). In some aspects, some of the memory pages (e.g., MEM2 and MEM3) may store calibration information and/or be for general data storage. In some aspects, the calibration information may include data related to converting measurements into physical units (e.g., amps, volts, and/or degrees Celsius). In some aspects, the calibration information may additionally or alternatively include behavioral calibration information, which may show how certain physical quantities vary with time (e.g., how the period or frequency of the clock 830 varies with the voltage VBAT produced by the charge storage device 202 and/or with temperature). In some aspects, sixteen of the memory pages (e.g., MEM4 to MEM19) may store autonomous sensor measurements. In some aspects, each of the sixteen memory pages of a sensing device may be capable of storing the sensor measurements of four measurement cycles. In some aspects, each memory page of a sensing device may be capable of storing the sensor measurements performed during four measurement cycles, and, with the sensing device performing two measurement cycles per autonomous sensor measurement (i.e., one measurement cycle for each of two sensing areas of the sensing device), the sixteen memory pages may be capable of storing sensor measurements of the sensing device for a total of 32 autonomous sensor measurements. In some alternative aspects, each memory 824 of a sensing device of the analyte sensor 100 may include twenty memory pages (e.g., MEM2 to MEM22), which may each be capable of storing the sensor measurements of four measurement cycles, and, with the sensing device performing two measurement cycles per autonomous sensor measurement (i.e., one measurement cycle for each of two sensing areas of the sensing device), the twenty memory pages may be capable of storing sensor measurements of the sensing device for a total of 40 autonomous sensor measurements.

In some aspects, as explained above, each of the autonomous sensor measurements may include a set of sensor measurements including one or more analyte measurements, one or more interferent measurements, one or more first reference measurements, one or more second reference measurements, one or more temperature measurements, one or more voltage measurements (e.g., one or more measurements of the voltage VBAT produced by the charge storage device 202), and/or timing information. In some aspects, the set of sensor measurements may include sensor measurements associated with one or more measurements cycles (e.g., first and second measurement cycles) for one or more sensing areas, respectively, of a sensing device. In some aspects, the sensor measurements associated with a first measurement cycle for a sensing area 2202a may include, for example and without limitation, 13 measurements. In some aspects, sensor measurements associated with the first measurement cycle for the sensing area 2202a of the first sensing device 100A may include, for example and without limitation, (1) a field current measurement value (e.g., Icouple) from the first sensing device 100A, (2) a first temperature measurement from the first sensing device 100A, (3) an ambient light measurement from the one or more signal photodetectors 224 in the sensing area 2202a (e.g., with the light sources 108 and 227 off), (4) an ambient light measurement from the one or more reference photodetectors 226 in the sensing area 2202a (e.g., with the light sources 108 and 227 off), (5) an ambient light measurement from the one or more interferent photodetectors 228 in the sensing area 2202a (e.g., with the light sources 108 and 227 off), (6) an ambient light measurement from the one or more second reference photodetectors 230 in the sensing area 2202a (e.g., with the light sources 108 and 227 off), (7) an analyte measurement from the one or more signal photodetectors 224 in the sensing area 2202a (e.g., with the first light source 108 on and the second light source 227 off), (8) a first reference measurement from the one or more reference photodetectors 226 in the sensing area 2202a (e.g., with the first light source 108 on and the second light source 227 off), (9) an interferent measurement from the one or more interferent photodetectors 228 in the sensing area 2202a (e.g., with the first light source 108 off and the second light source 227 on), (10) a second reference measurement from the one or more second reference photodetectors 230 in the sensing area 2202a (e.g., with the first light source 108 off and the second light source 227 on), (11) a measurement of an impedance of the first light source 108 in the sensing area 2202a, (12) a measurement of the impedance of the second light source 227 in the sensing area 2202a, and (13) a measurement by the first sensing device 100A of the voltage VBAT produced by the charge storage device 202. In some aspects, the sensor measurements associated with a second measurement cycle for a sensing area 2202c of the first sensing device 100A may include, for example and without limitation, 13 measurements. In some aspects, sensor measurements associated with the second measurement cycle for the sensing area 2202c may include, for example and without limitation, (1) a first diagnostic measurement, (2) a second diagnostic measurement, (3) an ambient light measurement from the one or more signal photodetectors 224 in the sensing area 2202c (e.g., with the light sources 108 and 227 off), (4) an ambient light measurement from the one or more reference photodetectors 226 in the sensing area 2202c (e.g., with the light sources 108 and 227 off), (5) an ambient light measurement from the one or more interferent photodetectors 228 in the sensing area 2202c (e.g., with the light sources 108 and 227 off), (6) an ambient light measurement from the one or more second reference photodetectors 230 in the sensing area 2202c (e.g., with the light sources 108 and 227 off), (7) an analyte measurement from the one or more signal photodetectors 224 in the sensing area 2202c (e.g., with the first light source 108 on and the second light source 227 off), (8) a first reference measurement from the one or more reference photodetectors 226 in the sensing area 2202c (e.g., with the first light source 108 on and the second light source 227 off), (9) an interferent measurement from the one or more interferent photodetectors 228 in the sensing area 2202c (e.g., with the first light source 108 off and the second light source 227 on), (10) a second reference measurement from the one or more second reference photodetectors 230 in the sensing area 2202c (e.g., with the first light source 108 off and the second light source 227 on), (11) a measurement of an impedance of the first light source 108 in the sensing area 2202c, (12) a measurement of the impedance of the second light source 227 in the sensing area 2202c, and (13) a second temperature measurement from the first sensing device 100A. In some aspects, the diagnostic measurements may include, for example and without limitation, timing information, such as, a count of the cycles of the clock 830 and/or a number n for the autonomous sensor measurement. In some aspects, the one or more measurement cycles associated with one or more sensing areas (e.g., sensing areas 2202b and 2202d) of the second sensing device 100B may be similar to the one or more measurement cycles associated with one or more sensing areas (e.g., sensing areas 2202a and 2202c) of the first sensing device 100A.

In some aspects in which the memories 824 of the analyte sensor 100 are capable of storing 40 autonomous sensor measurements, the memories 824 of the sensing devices of the analyte sensor 100 may have the configuration shown in FIG. 21B. In some aspects, as shown in FIG. 21B, a memory 824 of a sensing device of the analyte sensor 100 may have 22 memory pages (i.e., MEM0 to MEM21). In some aspects, each of the memory pages may include, for example and without limitation, sixty-four 16-bit registers. In some aspects, two of the memory pages (e.g., MEM0 and MEM1) may store configuration information (e.g., an identification of the analyte sensor 100, an identification of the sensing device of the analyte sensor 100, near field communication (NFC) information, first and second measurement cycle setup parameters, and general data storage). In some aspects, the NFC information may include may include settings for how the sensing device should demodulate/interpret incoming NFC signals (e.g., at a low level) and/or how the sensing device should respond to NFC signals (e.g., at a low level). In some aspects, the first and second measurement cycle setup parameters may include an automatic wait parameter indicative of whether the sensing device (e.g., sensing device 100a) of the analyte device 100 should delay for a period of time before performing an autonomous measurement sequence (e.g., to avoid interference with another sensing device, such as sensing device 100b, of the analyte sensor 100 that might occur if the sensing devices perform autonomous measurement sequences at the same time). In some aspects, the general data storage may include, for example and without limitation, part number information, system status information (e.g., insertion date and time), and/or health status of each measurement cycle. In some aspects, some of the memory pages (e.g., MEM2 to MEM6 as shown in FIG. 21B or spread across the last few registers of MEM2 through MEM21) may store calibration information, and/or some of the memory pages (e.g., MEM7 to MEM21) may be for general data storage. In some aspects, twenty of the memory pages (e.g., MEM2 to MEM21) may store autonomous sensor measurements. In some aspects, each of the twenty memory pages may store two autonomous sensor measurements for a total of 40 autonomous sensor measurements (i.e., 2×20=40). In some aspects, as explained above, each autonomous sensor measurements may include a set of sensor measurements including one or more analyte measurements, one or more interferent measurements, one or more first reference measurements, one or more second reference measurements, one or more temperature measurements, one or more voltage measurements (e.g., one or more measurements of the voltage VBAT produced by the charge storage device 202), and/or timing information. In some aspects, the sets of sensor measurements may each include, for example and without limitation, 13 measurements.

In some aspects, the analyte sensor 100 may store the most-recent autonomous sensor measurements (e.g., in a first-in-first-out (FIFO) fashion). For example, if the memories 824 of the sensing devices each store 40 autonomous sensor measurements as shown in FIG. 21B, the memories 824 may store the 40 most-recent autonomous sensor measurement (e.g., in MEM2 to MEM21) and may discard older autonomous sensor measurement. Thus, in some aspects in which the analyte sensor 100 takes autonomous sensor measurements at a regular interval of time/frequency of every 3 minutes, the memories 824 may store the most-recent 40 autonomous sensor measurements, which would cover a 1 hour and 57 minute span of time. In some aspects in which the analyte sensor 100 takes autonomous sensor measurements at a regular interval of time/frequency of every 5 minutes, the memories 824 may store the most-recent 40 autonomous sensor measurements, which would cover a 3 hour and 15 minute span of time. In some aspects in which the analyte sensor 100 takes autonomous sensor measurements at a regular interval of time/frequency of every 15 minutes, the memories 824 may store the most-recent 40 autonomous sensor measurements, which would cover a 9 hour and 45 minute span of time. For another example, if the memories 824 of the sensing devices of the analyte sensor 100 store 64 autonomous sensor measurements as shown in FIG. 21A, the memories 824 may store the 64 most-recent autonomous sensor measurement and may discard older autonomous sensor measurement (e.g., in some aspects which the analyte sensor 100 takes autonomous sensor measurements at a regular interval of time/frequency of every 5 minutes, the memories 824 may store the most-recent 64 autonomous sensor measurements, which would cover a 5 hour and 15 minute span of time).

In some alternative aspects, the analyte sensor 100 may store the most-recent autonomous sensor measurements at a first frequency, which may be the frequency at which the measurements are taken, and may store less-recent autonomous sensor measurements at a second frequency. In some aspects, the first frequency may be greater than the second frequency. In this way, the analyte sensor 100 may down sample the less-recent autonomous sensor measurements. In some aspects, the analyte sensor 100 may store the most-recent autonomous sensor measurements in a FIFO fashion with autonomous sensor measurements being added at the first frequency and may store the less-recent autonomous sensor measurements in a FIFO fashion with autonomous sensor measurements being added at the second frequency. In some aspects, the second frequency may be 1/Nth of the first frequency such that every Nth less-recent autonomous sensor measurement is stored. In this way, N may be the down sampling rate, and the down sampling rate N may be stored by the analyte sensor 100 (e.g., in the one or more memories 824 of the one or more sensing devices of the analyte sensor 100).

For example, if the memories 824 of the sensing devices each store 40 autonomous sensor measurements as shown in FIG. 21B, the memories 824 may store (i) the 8 most-recent autonomous sensor measurement at the first frequency (e.g., in MEM2 to MEM5) and (ii) 32 less-recent measurements at the second frequency (e.g., in MEM6 to MEM21). Thus, in some aspects in which the analyte sensor 100 takes autonomous sensor measurements at a regular interval of time/frequency of every 3 minutes, the memories 824 may store the most-recent 8 autonomous sensor measurements at the first frequency of every 3 minutes, which would cover a 21 minute span of time, and may store 32 less-recent autonomous sensor measurements at a second frequency of every 15 minutes (e.g., N=5), which would cover a 7 hour and 45 minute span of time, for a total time span of 8 hours and 11 minutes. In some aspects in which the analyte sensor 100 takes autonomous sensor measurements at a regular interval of time/frequency of every 5 minutes, the memories 824 may store the most-recent 8 autonomous sensor measurements, which would cover a 35 minute span of time, and may store 32 less-recent autonomous sensor measurements at a second frequency of every 15 minutes (e.g., N=3), which would cover a 7 hour and 45 minute span of time, for a total time span of 8 hours and 25 minutes. In some aspects in which the analyte sensor 100 takes autonomous sensor measurements at a regular interval of time/frequency of every 15 minutes, the memories 824 may store the most-recent 40 autonomous sensor measurements, which would cover a 9 hour and 45 minute span of time. For another example, if the memories 824 of the sensing devices of the analyte sensor 100 each store 64 autonomous sensor measurements as in FIG. 21A, the memories 824 may store (i) the 16 most-recent autonomous sensor measurement at the first frequency and (ii) 48 less-recent measurements at the second frequency.

FIGS. 21C and 21D show an example in which the one or more memories 824 of the analyte sensor 100 are capable of storing up to 40 autonomous sensor measurements, with Data Blocks 1-8 reserved for storing the four most-recent sensor measurements at a first interval of time and Down Sampled Data Blocks 1-32 reserved for storing six down-sampled, less-recent measurements at a second interval of time. In the example shown in FIGS. 21C and 21D, the down sampling rate N is 3. That is, every third less-recent sensor measurement is stored in a down-sampled data block instead of being discarded. As shown in FIG. 21C, in this example, at a first time t1 (e.g., at 0 minutes), the analyte sensor 100 autonomously takes a first set of sensor measurements Data[t1] and stores the first set of sensor measurements Data[t1] in Data Block 1. As shown in FIG. 21C, in this example, at a second time t2 (e.g., at 5 minutes), the analyte sensor 100 takes a second set of autonomous sensor measurements Data[t2] and stores the second set of sensor measurements Data[t2] in Data Block 2. In this example, after time t8 (e.g., 35 minutes), the eight most recent sensor measurements are stored in Data Blocks 1-8, respectively. Then, at time t9 (e.g., 40 minutes), the analyte sensor 100 begins down-sampling less-recent sets of sensor measurements and storing them at a second frequency (e.g., every third interval of time) in the Down Sampled Data Blocks 1-32. Accordingly, at time t9, the analyte sensor 100 (i) takes a ninth set of autonomous sensor measurements Data[t9], (ii) copies the first set of sensor measurements Data[t1] from Data Block 1 and stores it in Down Sampled Data Block 1, and (iii) stores the ninth set of sensor measurements Data[t9] in Data Block 1, which overwrites the first set of sensor measurements Data[t1] that was stored in Data Block 1. At time t10 (e.g., 45 minutes), the analyte sensor 100 takes a tenth set of autonomous sensor measurements Data[t10] and stores the tenth set of sensor measurements Data[t10] in Data Block 2, which overwrites and permanently erases the second set of sensor measurements Data[t2] that was previously stored in Data Block 2. At time t11 (e.g., 50 minutes), the analyte sensor 100 takes an eleventh set of autonomous sensor measurements Data[t11] and stores the eleventh set of sensor measurements Data[t11] in Data Block 3, which overwrites and permanently erases the third set of sensor measurements Data[t3] that was previously stored in Data Block 3. Therefore, in this example, every third less-recent set of sensor measurements (e.g., Data[t1], Data[t4], Data[7], Data[t10], Data[t13], etc.) is stored in the down-sampled data blocks, and the other less-recent set of sensor measurements (e.g., Data[t2], Data[t3], Data[t5], Data[t6], Data[t8], Data[t9], Data[t11], Data[t12], Data[t14], Data[t15], etc.) are discarded. In this example, at time t105, with Down Sampled Data Blocks 1-32 being full, the analyte sensor 100 (i) takes a 105th set of autonomous sensor measurements Data[t105], (ii) copies the 97^{th} set of sensor measurements Data[t97] from Data Block 1 and stores it in Down Sampled Data Block 1, which overwrites and erases the prior contents of Down Sampled Data Block 1, and (iii) stores the 105^{t}h set of autonomous sensor measurements Data[t105] in Data Block 1, which overwrites the prior contents of Data Block 1.

FIG. 21D shows the contents of the data blocks with the relative measurement times for the example shown in FIG. 21C assuming that the first interval of time is five minutes. That is, FIG. 21D shows the relative measurements times for the example assuming that the analyte sensor 100 takes and stores sets of sensor measurements at a first frequency of every five minutes. In FIG. 21D (as in FIG. 21C), the down sampling rate N is 3. That is, every third less-recent sensor measurement is stored in a down-sampled data block instead of being discarded. As shown in FIG. 21D, in this example, after a time t equal to 35 minutes after the first time t1 (i.e., t=t1+35), the eight most recent sensor measurements (i.e., Data[t-35 minutes], Data[t-30 minutes], Data[t-25 minutes], Data[t-20 minutes], Data[t-15 minutes], Data[t-10 minutes], Data[t-5 minutes], and Data[t]) are stored in Data Blocks 1-8, respectively. Then, at time t equal to 40 minutes after the first time (i.e., t=t1+40), the analyte sensor 100 begins down-sampling less-recent sets of sensor measurements and storing them at a second frequency (e.g., every third interval of time) in the Down Sampled Data Blocks 1-32. Accordingly, at time t=t1+40, the analyte sensor 100 (i) takes a ninth set of autonomous sensor measurements Data[t], which is the same as Data[t1+40], (ii) copies the first set of sensor measurements (i.e., Data[t-40], which is the same as Data[t1]), which was taken 40 minutes prior to the current time t=t1+40, from Data Block 1 and stores it in Down Sampled Data Block 1, and (iii) stores the ninth set of sensor measurements Data[t] in Data Block 1, which overwrites the first set of sensor measurements Data[t-40] previously stored in Data Block 1. At time at time t=t1+45 minutes, the analyte sensor 100 (i) takes a tenth set of autonomous sensor measurements Data[t], which is the same as Data[t1+45], and (ii) stores the tenth set of sensor measurements Data[t] in Data Block 2, which overwrites and permanently erases the second set of sensor measurements (i.e., Data[t-40], which is the same as Data[t1+5]) that was previously stored in Data Block 2. At time t=t1+50 minutes, the analyte sensor 100 (i) takes an eleventh set of autonomous sensor measurements Data[t], which is the same as Data[t1+50], and (ii) stores the eleventh set of sensor measurements Data[t] in Data Block 3, which overwrites and permanently erases the third set of sensor measurements (i.e., Data[t-40], which is the same as Data[t1+10]) that was previously stored in Data Block 3. Therefore, in this example, every third less-recent set of sensor measurements is stored in the down-sampled data blocks, and the other less-recent set of sensor measurements are discarded. In this example, at time t=t1+520 minutes, with Down Sampled Data Blocks 1-32 being full, the analyte sensor 100 (i) takes a 105^{th} set of autonomous sensor measurements Data[t], which is the same as Data[t1+520], (ii) copies the 97^{t}h set of sensor measurements (i.e., Data[t-40], which is the same as Data[t1+480]), which was taken 40 minutes prior to the current time t=t1+520, from Data Block 1 and stores it in Down Sampled Data Block 1, which overwrites and permanently erases the first set of sensor measurements Data[t-520] previously stored in Down Sampled Data Block 1, and (iii) stores the 105^{t}h set of sensor measurements Data[t] in Data Block 1, which overwrites the 97^{t}h set of sensor measurements Data[t-40] that was stored in Data Block 1.

Table 2 below shows a different example in which the one or more memories 824 of the analyte sensor 100 are capable of storing up to 10 autonomous sensor measurements, with four memory addresses (e.g., A0-A3) reserved for storing the four most-recent sensor measurements at a 5-minute interval of time and six memory addresses (e.g., A4-A9) reserved for storing six down-sampled, less-recent measurements at a 15-minute interval of time. As shown in Table 2, in this example, at a time TO (e.g., at 0 minutes), the analyte sensor 100 autonomously takes a set of sensor measurements M0 and stores the set of sensor measurements M0 at memory address A0. As shown in Table 2, in this example, at a time T5 (e.g., at 5 minutes), the analyte sensor 100 takes a set of autonomous sensor measurements M1, shifts the set of sensor measurements M0 from memory address A0 to memory address A1, and stores the set of sensor measurements M1 at memory address A0. In this example, after time T15 (e.g., 15 minutes) at which the four most recent sensor measurements are stored in memory addresses A0-A3, respectively, the analyte sensor 100 begins down-sampling less-recent sensor measurements and storing them at a 15-minnute interval of time. Accordingly, in this example, the set of sensor measurements M0 is stored until time T110, but the sets of sensor measurements M5 and M10 are discarded at times T25 and T30, respectively. In this example, the set of sensor measurements M0 is discarded at time T110 to make room for sets of sensor measurements M15, M30, M45, M60, M75, and M90.

TABLE 2

| | Down-Sampled, Less-Recent Measurements | | | | | | Most-Recent Measurements | | | |
|---|---|---|---|---|---|---|---|---|---|---|
| Time | A9 | A8 | A7 | A6 | A5 | A4 | A3 | A2 | A1 | A0 |
| T0 | — | — | — | — | — | — | — | — | — | M0 |
| T5 | — | — | — | — | — | — | — | — | M0 | M5 |
| T10 | — | — | — | — | — | — | — | M0 | M5 | M10 |
| T15 | — | — | — | — | — | — | M0 | M5 | M10 | M15 |
| T20 | — | — | — | — | — | M0 | M5 | M10 | M15 | M20 |
| T25 | — | — | — | — | — | M0 | M10 | M15 | M20 | M25 |
| T30 | — | — | — | — | — | M0 | M15 | M20 | M25 | M30 |
| T35 | — | — | — | — | M0 | M15 | M20 | M25 | M30 | M35 |
| T40 | — | — | — | — | M0 | M15 | M25 | M30 | M35 | M40 |
| T45 | — | — | — | — | M0 | M15 | M30 | M35 | M40 | M45 |
| T50 | — | — | — | M0 | M15 | M30 | M35 | M40 | M45 | M50 |
| T55 | — | — | — | M0 | M15 | M30 | M40 | M45 | M50 | M55 |
| T60 | — | — | — | M0 | M15 | M30 | M45 | M50 | M55 | M60 |
| T65 | — | — | M0 | M15 | M30 | M45 | M50 | M55 | M60 | M65 |
| T70 | — | — | M0 | M15 | M30 | M45 | M55 | M60 | M65 | M70 |
| T75 | — | — | M0 | M15 | M30 | M45 | M60 | M65 | M70 | M75 |
| T80 | — | M0 | M15 | M30 | M45 | M60 | M65 | M70 | M75 | M80 |
| T85 | — | M0 | M15 | M30 | M45 | M60 | M70 | M75 | M80 | M85 |
| T90 | — | M0 | M15 | M30 | M45 | M60 | M75 | M80 | M85 | M90 |
| T95 | M0 | M15 | M30 | M45 | M60 | M75 | M80 | M85 | M90 | M95 |
| T100 | M0 | M15 | M30 | M45 | M60 | M75 | M85 | M90 | M95 | M100 |
| T105 | M0 | M15 | M30 | M45 | M60 | M75 | M90 | M95 | M100 | M105 |
| T110 | M15 | M30 | M45 | M60 | M75 | M90 | M95 | M100 | M105 | M110 |
| T115 | M15 | M30 | M45 | M60 | M75 | M90 | M100 | M105 | M110 | M115 |
| T120 | M15 | M30 | M45 | M60 | M75 | M90 | M105 | M110 | M115 | M120 |
| T125 | M30 | M45 | M60 | M75 | M90 | M105 | M110 | M115 | M120 | M125 |
| T130 | M30 | M45 | M60 | M75 | M90 | M105 | M115 | M120 | M125 | M130 |
| T135 | M30 | M45 | M60 | M75 | M90 | M105 | M120 | M125 | M130 | M135 |
| T140 | M45 | M60 | M75 | M90 | M105 | M120 | M125 | M130 | M135 | M140 |
| T145 | M45 | M60 | M75 | M90 | M105 | M120 | M130 | M135 | M140 | M145 |
| T150 | M45 | M60 | M75 | M90 | M105 | M120 | M135 | M140 | M145 | M150 |
| T155 | M60 | M75 | M90 | M105 | M120 | M135 | M140 | M145 | M150 | M155 |
| T160 | M60 | M75 | M90 | M105 | M120 | M135 | M145 | M150 | M155 | M160 |

Table 3 below shows another example in which the one or more memories 824 of the analyte sensor 100 are capable of storing up to 10 autonomous sensor measurements, with four memory addresses (e.g., A0-A3) reserved for storing the four most-recent sensor measurements at a 5-minute interval of time and six memory addresses (e.g., A4-A9) reserved for storing six down-sampled, less-recent measurements at a 15-minute interval of time. However, the example shown in Table 3 is different than the example shown in Table 2 because the analyte sensor 100 does not start down-sampling less-recent measurements until after all the memory addresses (e.g., memory addresses A0-A9) are full. Accordingly, in this example, the sets of sensor measurements M5 and M10 are discarded at times T50 and T55, respectively.

TABLE 3

| | Down-Sampled, Less-Recent Measurements | | | | | | Most-Recent Measurements | | | |
|---|---|---|---|---|---|---|---|---|---|---|
| | A9 | A8 | A7 | A6 | A5 | A4 | A3 | A2 | A1 | A0 |
| T0 | — | — | — | — | — | — | — | — | — | M0 |
| T5 | — | — | — | — | — | — | — | — | M0 | M5 |
| T10 | — | — | — | — | — | — | — | M0 | M5 | M10 |
| T15 | — | — | — | — | — | — | M0 | M5 | M10 | M15 |
| T20 | — | — | — | — | — | M0 | M5 | M10 | M15 | M20 |
| T25 | — | — | — | — | M0 | M5 | M10 | M15 | M20 | M25 |
| T30 | — | — | — | M0 | M5 | M10 | M15 | M20 | M25 | M30 |
| T35 | — | — | M0 | M5 | M10 | M15 | M20 | M25 | M30 | M35 |
| T40 | — | M0 | M5 | M10 | M15 | M20 | M25 | M30 | M35 | M40 |
| T45 | M0 | M5 | M10 | M15 | M20 | M25 | M30 | M35 | M40 | M45 |
| T50 | M0 | M10 | M15 | M20 | M25 | M30 | M35 | M40 | M45 | M50 |
| T55 | M0 | M15 | M20 | M25 | M30 | M35 | M40 | M45 | M50 | M55 |
| T60 | M0 | M15 | M25 | M30 | M35 | M40 | M45 | M50 | M55 | M60 |
| T65 | M0 | M15 | M30 | M35 | M40 | M45 | M50 | M55 | M60 | M65 |
| T70 | M0 | M15 | M30 | M40 | M45 | M50 | M55 | M60 | M65 | M70 |
| T75 | M0 | M15 | M30 | M45 | M50 | M55 | M60 | M65 | M70 | M75 |
| T80 | M0 | M15 | M30 | M45 | M55 | M60 | M65 | M70 | M75 | M80 |
| T85 | M0 | M15 | M30 | M45 | M60 | M65 | M70 | M75 | M80 | M85 |
| T90 | M0 | M15 | M30 | M45 | M60 | M70 | M75 | M80 | M85 | M90 |
| T95 | M0 | M15 | M30 | M45 | M60 | M75 | M80 | M85 | M90 | M95 |
| T100 | M0 | M15 | M30 | M45 | M60 | M75 | M85 | M90 | M95 | M100 |
| T105 | M0 | M15 | M30 | M45 | M60 | M75 | M90 | M95 | M100 | M105 |
| T110 | M15 | M30 | M45 | M60 | M75 | M90 | M95 | M100 | M105 | M110 |
| T115 | M15 | M30 | M45 | M60 | M75 | M90 | M100 | M105 | M110 | M115 |
| T120 | M15 | M30 | M45 | M60 | M75 | M90 | M105 | M110 | M115 | M120 |
| T125 | M30 | M45 | M60 | M75 | M90 | M105 | M110 | M115 | M120 | M125 |
| T130 | M30 | M45 | M60 | M75 | M90 | M105 | M115 | M120 | M125 | M130 |
| T135 | M30 | M45 | M60 | M75 | M90 | M105 | M120 | M125 | M130 | M135 |
| T140 | M45 | M60 | M75 | M90 | M105 | M120 | M125 | M130 | M135 | M140 |
| T145 | M45 | M60 | M75 | M90 | M105 | M120 | M130 | M135 | M140 | M145 |
| T150 | M45 | M60 | M75 | M90 | M105 | M120 | M135 | M140 | M145 | M150 |
| T155 | M60 | M75 | M90 | M105 | M120 | M135 | M140 | M145 | M150 | M155 |
| T160 | M60 | M75 | M90 | M105 | M120 | M135 | M145 | M150 | M155 | M160 |

Table 4 below shows yet another example in which the one or more memories 824 of the analyte sensor 100 are capable of storing up to 10 autonomous sensor measurements, the four most-recent sensor measurements are stored at a 5-minute interval of time, and six down-sampled, less-recent measurements are stored at a 15-minute interval of time. However, in this example, memory addresses are not reserved for storing the most-recent sensor measurements at the 5-minute interval of time or the down-sampled, less-recent measurements at the 15-minute interval of time, and, instead of shifting stored sets of sensor measurements as new sets of sensor measurements come in, the sets of sensor measurements stay at one memory address until it is discarded.

TABLE 4

| | A9 | A8 | A7 | A6 | A5 | A4 | A3 | A2 | A1 | A0 |
|---|---|---|---|---|---|---|---|---|---|---|
| T0 | M0 | — | — | — | — | — | — | — | — | — |
| T5 | M0 | M5 | — | — | — | — | — | — | — | — |
| T10 | M0 | M5 | M10 | — | — | — | — | — | — | — |
| T15 | M0 | M5 | M10 | M15 | — | — | — | — | — | — |
| T20 | M0 | M5 | M10 | M15 | M20 | — | — | — | — | — |
| T25 | M0 | M5 | M10 | M15 | M20 | M25 | — | — | — | — |
| T30 | M0 | M5 | M10 | M15 | M20 | M25 | M30 | — | — | — |
| T35 | M0 | M5 | M10 | M15 | M20 | M25 | M30 | M35 | — | — |
| T40 | M0 | M5 | M10 | M15 | M20 | M25 | M30 | M35 | M40 | — |
| T45 | M0 | M5 | M10 | M15 | M20 | M25 | M30 | M35 | M40 | M45 |
| T50 | M0 | M50 | M10 | M15 | M20 | M25 | M30 | M35 | M40 | M45 |
| T55 | M0 | M50 | M55 | M15 | M20 | M25 | M30 | M35 | M40 | M45 |
| T60 | M0 | M50 | M55 | M15 | M60 | M25 | M30 | M35 | M40 | M45 |
| T65 | M0 | M50 | M55 | M15 | M60 | M65 | M30 | M35 | M40 | M45 |
| T70 | M0 | M50 | M55 | M15 | M60 | M65 | M30 | M70 | M40 | M45 |
| T75 | M0 | M50 | M55 | M15 | M60 | M65 | M30 | M70 | M75 | M45 |
| T80 | M0 | M80 | M55 | M15 | M60 | M65 | M30 | M70 | M75 | M45 |
| T85 | M0 | M80 | M85 | M15 | M60 | M65 | M30 | M70 | M75 | M45 |
| T90 | M0 | M80 | M85 | M15 | M60 | M90 | M30 | M70 | M75 | M45 |
| T95 | M0 | M80 | M85 | M15 | M60 | M90 | M30 | M95 | M75 | M45 |
| T100 | M0 | M100 | M85 | M15 | M60 | M90 | M30 | M95 | M75 | M45 |
| T105 | M0 | M100 | M105 | M15 | M60 | M90 | M30 | M95 | M75 | M45 |
| T110 | M110 | M100 | M105 | M15 | M60 | M90 | M30 | M95 | M75 | M45 |
| T115 | M110 | M100 | M105 | M15 | M60 | M90 | M30 | M115 | M75 | M45 |
| T120 | M110 | M120 | M105 | M15 | M60 | M90 | M30 | M115 | M75 | M45 |
| T125 | M110 | M120 | M105 | M125 | M60 | M90 | M30 | M115 | M75 | M45 |
| T130 | M130 | M120 | M105 | M125 | M60 | M90 | M30 | M115 | M75 | M45 |
| T135 | M130 | M120 | M105 | M125 | M60 | M90 | M30 | M135 | M75 | M45 |
| T140 | M130 | M120 | M105 | M125 | M60 | M90 | M140 | M135 | M75 | M45 |
| T145 | M130 | M120 | M105 | M145 | M60 | M90 | M140 | M135 | M75 | M45 |
| T150 | M150 | M120 | M105 | M145 | M60 | M90 | M140 | M135 | M75 | M45 |
| T155 | M150 | M120 | M105 | M145 | M60 | M90 | M140 | M135 | M75 | M155 |
| T160 | M150 | M120 | M105 | M145 | M60 | M90 | M160 | M135 | M75 | M155 |

Table 5 below shows the sets of measurements stored at the different intervals of time in the examples shown in Tables 3 and 4 above.

TABLE 5

| | Measurements Stored at 15 | Measurements Stored at 5 |
|---|---|---|
| | Minute Interval of Time | Minute Interval of Time |
| T0 | — | M 0 |
| T5 | — | M 0, M 5 |
| T10 | — | M 0, M 5, M 10 |
| T15 | — | M 0, M 5, M 10, M 15 |
| T20 | — | M 0, M 5, M 10, M 15, M 20 |
| T25 | — | M 0, M 5, M 10, M 15, M 20, |
| | | M 25 |
| T30 | — | M 0, M 5, M 10, M 15, M 20, |
| | | M 25, M 30 |
| T35 | — | M 0, M 5, M 10, M 15, M 20, |
| | | M 25, M 30, M 35 |
| T40 | — | M 0, M 5, M 10, M 15, M 20, |
| | | M 25, M 30, M 35, M 40 |
| T45 | — | M 0, M 5, M 10, M 15, M 20, |
| | | M 25, M 30, M 35, M 40, M 45 |
| T50 | M 0 | M 10, M 15, M 20, M 25, M 30, |
| | | M 35, M 40, M 45, M 50 |
| T55 | M 0 | M 15, M 20, M 25, M 30, M 35, |
| | | M 40, M 45, M 50, M 55 |
| T60 | M 0, M 15 | M 25, M 30, M 35, M 40, M 45, |
| | | M 50, M 55, M 60 |
| T65 | M 0, M 15 | M 30, M 35, M 40, M 45, M 50, |
| | | M 55, M 60, M 65 |
| T70 | M 0, M 15, M 30 | M 40, M 45, M 50, M 55, M 60, |
| | | M 65, M 70 |
| T75 | M 0, M 15, M 30 | M 45, M 50, M 55, M 60, M 65, |
| | | M 70, M 75 |
| T80 | M 0, M 15, M 30, M 45 | M 55, M 60, M 65, M 70, M 75, |
| | | M 80 |
| T85 | M 0, M 15, M 30, M 45 | M 60, M 65, M 70, M 75, M 80, |
| | | M 85 |
| T90 | M 0, M 15, M 30, M 45, M 60 | M 70, M 75, M 80, M 85, M 90 |
| T95 | M 0, M 15, M 30, M 45, M 60, | M 80, M 85, M 90, M 95 |
| | M 75 | |
| T100 | M 0, M 15, M 30, M 45, M 60, | M 85, M 90, M 95, M 100 |
| | M 75 | |
| T105 | M 0, M 15, M 30, M 45, M 60, | M 90, M 95, M 100, M 105 |
| | M 75 | |
| T110 | M 15, M 30, M 45, M 60, M 75, | M 95, M 100, M 105, M 110 |
| | M 90 | |
| T115 | M 15, M 30, M 45, M 60, M 75, | M 100, M 105, M 110, M 115 |
| | M 90 | |
| T120 | M 15, M 30, M 45, M 60, M 75, | M 105, M 110, M 115, M 120 |
| | M 90 | |
| T125 | M 30, M 45, M 60, M 75, M 90, | M 110, M 115, M 120, M 125 |
| | M 105 | |
| T130 | M 30, M 45, M 60, M 75, M 90, | M 115, M 120, M 125, M 130 |
| | M 105 | |
| T135 | M 30, M 45, M 60, M 75, M 90, | M 120, M 125, M 130, M 135 |
| | M 105 | |
| T140 | M 45, M 60, M 75, M 90, | M 125, M 130, M 135, M 140 |
| | M 105, M 120 | |
| T145 | M 45, M 60, M 75, M 90, | M 130, M 135, M 140, M 145 |
| | M 105, M 120 | |
| T150 | M 45, M 60, M 75, M 90, | M 135, M 140, M 145, M 150 |
| | M 105, M 120 | |
| T155 | M 60, M 75, M 90, M 105, | M 140, M 145, M 150, M 155 |
| | M 120, M 135 | |
| T160 | M 60, M 75, M 90, M 105, | M 145, M 150, M 155, M 160 |
| | M 120, M 135 | |

In some aspects in which the one or more sensing devices of the analyte sensor 100 take autonomous sensor measurements, when the transceiver 101 and/or the display device 105 receives sensor measurements from the analyte sensor 100, the transceiver 101 and/or the display device 105 may receive autonomous sensor measurements, which were stored in the one or more memories 824 of the analyte sensor 100. In some aspects, because the transceiver 101 and/or the display device 105 receives the autonomous sensor measurements from the sensing devices (e.g., sensing devices 100a and 100b) of the analyte sensor 100, the transceiver 101 and/or the display device 105 may receive the autonomous sensor measurements from the multiple sensing areas 2202 (e.g., sensing areas 2202a, 2202b, 2202c, and 2202d) of the analyte sensor 100. In some aspects, when calculating an analyte concentration (e.g., glucose concentration) for a given instance of time, the analyte monitoring system 50 may calculate individual analyte concentrations for each sensing area 2202 and a combined analyte concentration (e.g., based on a weighted average of the individual analyte concentrations). In some aspects, the analyte monitoring system 50 may use sensing area-specific health metrics that assess noise, foreign body response (FBR) degradation (e.g., as measured using interferent indicators 209), and/or stability of reference channels. In some aspects, the analyte monitoring system 50 may determine the quality of each of the sensing areas 2202a, 2202b, 2202c, and 2202d and selectively de-weighting underperforming areas (such as sensing area 2202d) when calculating the combined analyte concentration.

In some aspects, the transceiver 101 or the display device 105 of the analyte monitoring system 50 may start autonomous measurements by the analyte sensor 100 by conveying a start autonomous measurement command, which may be received by the analyte sensor 100 (e.g., by the sensing devices of the analyte sensor 100). In some aspects, upon receiving an autonomous measurement command, a measurement scheduler 328 of a sensing device of the analyte sensor 100 may start counting the cycles of the clock 830 and initiate measurement sequences at a first frequency (e.g., every time a number of cycles of the clock 830 that is approximately equal to a interval of time, such as, for example and without limitation, 3 minutes, 5 minutes, or 15 minutes). In some aspects, the first frequency at which the analyte sensor 100 takes autonomous measurements may be programmable (e.g., may be set by the transceiver 101 or the display device 105). In some aspects, to read data from the analyte sensor 100, the transceiver 101 or the display device 105 may (i) convey a command to stop autonomous measurements on each sensing device of that analyte sensor 100, (ii) read data stored in the memory 824 of each sensing device, and (iii) convey a command to restart autonomous measurements.

In some aspects, the frequency/cycles of the clock 830 of a sensing device of the analyte sensor 100 may be voltage and/or temperature dependent. In some aspects, the measurement scheduler 328 may count the temperature dependent cycles of the clock 830 to determine when to take autonomous measurements, and the frequency of the autonomous measurements taken by the analyte sensor 100 may change as the voltage supplied to the clock 830 changes and/or the temperature of the clock 830 changes. In some aspects, because the cycles of the clock 830 and, therefore, the timing of the autonomous measurements are voltage and/or temperature dependent, the transceiver 101 or the display device 105 may calculate time stamps for the autonomous measurements. In some aspects, the time stamps for autonomous measurements may be calculated based on (i) the frequency (e.g., programmed frequency) at which the analyte sensor 100 takes autonomous measurements, (ii) timing information for the autonomous measurements, (iii) the temperatures of the sensing device(s) of the analyte sensor 100 (e.g., throughout the history of measurements since the start of autonomous mode), (iv) a characterization of the temperature dependence of the cycles of the clock 830, (v) the voltages VBAT supplied by the charge storage device 202 to the clock(s) 830 of the sensing device(s) of the analyte sensor 100, and/or (vi) a characterization of the voltage dependence of the cycles of the clock 830. In some aspects, the timing information may include counts of cycles of the clock 830 at the times of the autonomous measurement were taken. In some aspects, the timing information may additionally or alternatively include a number n for the autonomous sensor measurement (e.g., indicating that the autonomous sensor measurement is the nth autonomous sensor measurement taken since autonomous measurements were started). In some aspects, the characterization of the temperature dependence of the cycles of the clock 830 may be measured/obtained during manufacturing of the sensing device of the analyte sensor 100. In some aspects, the characterization of the voltage dependence of the cycles of the clock 830 may be measured/obtained during manufacturing of the sensing device of the analyte sensor 100.

In some aspects, a communication sequence for reading data from the analyte sensor 100 may include the transceiver 101 or the display device 105 conveying (and the sensing devices of the analyte sensor 100 receiving) an inventory command to identify the sensing devices of the analyte sensor 100. In some aspects, in response to receiving an inventory command, each of the sensing devices (e.g., sensing device 100a and 100b) of the analyte sensor 100 may convey an identification of the sensing device, which may be received by the transceiver 101 or the display device 105. In some aspects, the communication sequence may include the transceiver 101 or the display device 105 conveying (and the sensing devices of the analyte sensor 100 receiving) one or more commands to stop autonomous measurement by the sensing devices. In some aspects, the transceiver 101 or the display device 105 may convey a single unaddressed stop measurement command to all the sensing devices of the analyte sensor 100. In some alternative aspects, the transceiver 101 or the display device 105 may convey, for each of the sensing devices of the analyte sensor 100, a stop measurement command addressed to the sensing device. In some aspects, in response to receiving a stop measurement command, a sensing device of the analyte sensor 100 may convey a count of the cycles of the clock 830 of the sensing device since the sensing device last took an autonomous measurement and the autonomous measurements stored in the memory 824 of the sensing device (e.g., the autonomous measurements stored in memory pages MEM2 to MEM21 of a memory 824 having the configuration shown in FIG. 21). In some aspects, the communication sequence may include the transceiver 101 or the display device 105 conveying (and the sensing devices of the analyte sensor 100 receiving) one or more commands to start autonomous measurement by the sensing devices. In some aspects, the transceiver 101 or the display device 105 may convey a single unaddressed start measurement command to all the sensing devices of the analyte sensor 100. In some alternative aspects, the transceiver 101 or the display device 105 may convey, for each of the sensing devices of the analyte sensor 100, a start measurement command addressed to the sensing device.

In some aspects, the transceiver 101, display device 105, or the DMS 121 may calculate analyte concentrations (e.g., glucose concentrations) using the received autonomous measurements. In some aspects, calculating analyte concentrations may include (i) calculating interstitial fluid analyte concentrations based on the autonomous sensor measurements, (ii) calculating interstitial fluid analyte concentration rates-of-change based on the interstitial analyte concentrations, and (iii) calculating blood analyte concentrations based on the calculated interstitial analyte concentrations and the calculated interstitial fluid analyte concentration rates-of-change. In some aspects, calculating the interstitial fluid analyte concentration rate-of-change for the most-recent interstitial fluid analyte concentration may be based on the most-recent interstitial fluid analyte concentration and one or more less-recent interstitial fluid analyte concentrations (e.g., using a causal method with “backward difference” derivative calculation). In some aspects, calculating the interstitial fluid analyte concentration rate-of-change for an interstitial fluid analyte concentration other than the most-recent interstitial fluid analyte concentration may be based on the historical interstitial fluid analyte concentration, one or more less recent interstitial fluid analyte concentrations, and one or more recent interstitial fluid analyte concentrations (e.g., using an acausal centered difference for derivative calculations).

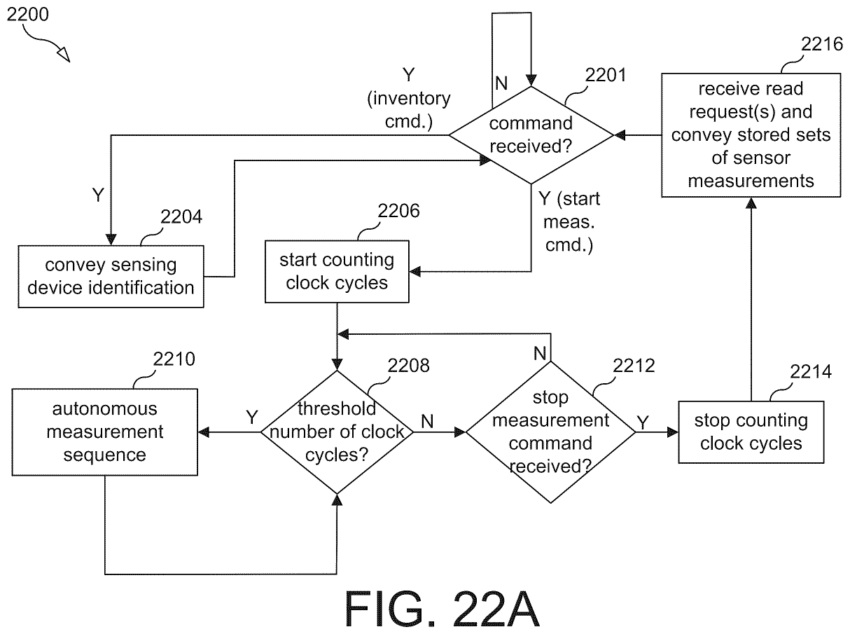

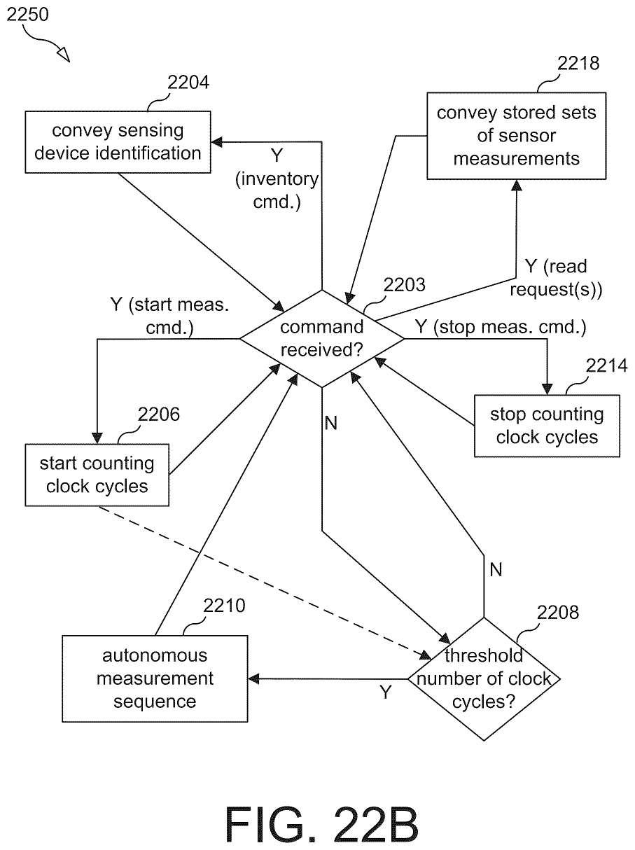

FIG. 22A is a flowchart illustrating a process 2200 that may be performed by the analyte monitoring system 50 according to some aspects. In some aspects, one or more steps of the process 2200 may be performed by an apparatus (e.g., the analyte sensor 100) of the analyte monitoring system 50. In some aspects, one or more steps of the process 2200 may be performed by a sensing device (e.g., sensing device 100A or 100B) of the analyte sensor 100. In some aspects in which the analyte sensor 100 includes two or more sensing devices, each of the sensing devices of the analyte sensor 100 may perform the process 2200. In some aspects, circuitry (e.g., mounted and/or fabricated on a substrate 112) of sensing device of the analyte sensor 100 may perform one or more steps of the process 2200.

In some aspects, as shown in FIG. 22A, the process 2200 may include a step 2201 in which the analyte sensor 100 (e.g., a sensing device of the analyte sensor 100) determines whether a command has been received. In some aspects, the command may be conveyed by an external device (e.g., the transceiver 101 or the display device 105). In some aspects, a sensing device of the analyte sensor 100 may receive the command via the antenna 114. In some aspects, a sensing device receiving a command may include a data extractor 444 extracting data from an alternating current produced by antenna 114, a decoder of the I/O digital circuitry 334 decoding extracted data, and/or a command decoder 322 decoding the command from the decoded data. In some aspects, if no command has been received in step 2201, the process 2200 may stay in step 2201 until a command is received.

In some aspects, if a command has been received in step 2201, and the command is an inventory command, the process 2200 may proceed from step 2201 to a step 2204. In some aspects, in step 2204, in response to receiving an inventory command, the sensing device of the analyte sensor 100 may convey an identification of the sensing device, which may be received by the external device. In some aspects, if a command has been received in step 2201, and the command is a start measurement command, the process 2200 may proceed from step 2201 to a step 2206.

In some aspects, as shown in FIG. 22A, the process 2200 may include the step 2206. In some aspects, in step 2206, if a sensing device of the analyte sensor 100 has received a start measurement command in step 2201, the sensing device (e.g., the measurement scheduler 328 of the sensing device) may start counting the cycles of the clock 830. In some aspects, the step 2206 may include the sensing device resetting the count of the cycles of the clock 830 (e.g., to zero) before starting to count the cycles of the clock 830. In some aspects, as shown in FIG. 22A, the process 2200 may include a step 2208 in which the sensing device (e.g., the measurement scheduler 328 of the sensing device) determines whether the sensing device (e.g., the measurement scheduler 328 of the sensing device) has counted of a threshold number of cycles of the clock 830. In some aspects, if the sensing device determines that the sensing device has not counted the threshold number of cycles of the clock 830, the process 2200 may proceed from step 2208 to a step 2212 in which the sensing device determines whether a stop autonomous measurement command has been received, and, if not, the process 2200 proceeds back to the step 2208. In some aspects, if the sensing device determines that the sensing device has counted the threshold number of cycles of the clock 830, the process 2200 may proceed from step 2208 to a step 2210.

In some aspects, as shown in FIG. 22A, the process 2200 may include the step 2210. In some aspects, in step 2210, the sensing device may perform a measurement sequence. In some aspects, the measurement sequence may be an autonomous measurement sequence because the sensing device determines on its own when to perform the autonomous measurement sequence. In some aspects, the measurement scheduler 328 of the sensing device may initiate (and the sensing device may perform) measurement sequences at a first frequency, which may be every time the measurement scheduler 328 counts the threshold number of cycles of the clock 830. In some aspects, the threshold number of cycles of the clock 830 may be approximately equal to an interval of time, such as, for example and without limitation, 3 minutes, 5 minutes, or 15 minutes.

In some aspects, the autonomous measurement sequence performed by the sensing device of the analyte sensor 100 may produce a set of sensor measurements including one or more analyte measurements (e.g., indicative of the amount of first emission light 331 emitted by the analyte indicator 207 and received by one or more signal photodetectors 224), one or more interferent measurements (e.g., indicative of the amount of second emission light 332 emitted by the interferent indicator 209 and received by the one or more interferent photodetectors 228), one or more first reference measurements (e.g., indicative of the level of first excitation light 329 reflected from the indicator element 106 and received by the one or more reference photodetectors 226), one or more second reference measurements (e.g., indicative of the level of second excitation light 330 reflected from the indicator element 106 and received by the one or more signal photodetectors 224 or the one or more second reference photodetectors 230), one or more temperature measurements (e.g., generated by a temperature transducer 464 or 492 of the sensor elements 832), one or more voltage measurements (e.g., one or more measurements of the voltage VBAT produced by the charge storage device 202, which may be generated by the CSD monitor 466 and digitized by the ADC 482), and/or timing information. In some aspects in which the sensing device includes one sensor area, the set of sensor measurements may include sensor measurements from one sensing area. In some alternative aspects in which the sensing device includes more than one sensing area, the set of sensor measurements may include sensor measurements from more than one sensing area (e.g., from sensing areas 2202a and 2202c of sensing device 100A or sensing areas 2202b and 2202d of sensing device 100B).

In some aspects, the step 2210 may include the sensing device storing the set of sensor measurements (e.g., in memory 824). In some aspects, in step 2210, if the memory 824 is full (e.g., if the pages of the memory 824 that store sets of sensor measurements are full), the sensing device of the analyte sensor 100 may store the just-produced set of sensor measurements in the memory 824 and discard the oldest set of sensor measurements from the memory 824 (e.g., in a first-in-first-out (FIFO) fashion). In some alternative aspects, in step 2210, if the memory 824 is full (e.g., if the pages of the memory 824 that store sets of sensor measurements are full), the sensing device of the analyte sensor 100 may store the just-produced set of sensor measurements in the memory 824 and discard a less-recent set of sensor measurements such that memory 824 stores the most-recent autonomous sensor measurements at a first frequency and stores less-recent autonomous sensor measurements at a second frequency. In some aspects, the first frequency at which the memory 824 stores the most-recent sets of sensor measurements may be the frequency at which the measurements are taken. In some aspects, the first frequency may be every time the measurement scheduler 328 counts the threshold number of cycles of the clock 830 (as identified in step 2208) and the sensing device performs a measurement sequence in step 2210. In some aspects, the first frequency may be greater than the second frequency. In this way, the sensing device may down sample the less-recent sets of sensor measurements. In some aspects, the sensing device may store the most-recent sets of sensor measurements in a FIFO fashion with sets of sensor measurements being added at the first frequency and may store the less-recent autonomous sensor measurements in a FIFO fashion with sets of sensor measurements being added at the second frequency. In some aspects, (i) the memory 824 acts as if it has a first FIFO for the most-recent sets of sensor measurements and a second FIFO for less-recent sets of sensor measurements, (ii) each time step 2210 is performed and the sensing device produces a set of sensor measurements, the sensing device adds the just-produced set of sensor measurements to the first FIFO and discards from the first FIFO the oldest set of sensor measurements that was in the first FIFO, and (iii) every Xth (e.g., every 2^{nd}, 3^{rd}, 4^{th}, 5^{th}, 6^{th}, 7^{th} 8^{th}, 9^{th}, or 10^{th}) time step 2210 is performed, the sensing device adds the set of sensor measurements discarded from the first FIFO to the second FIFO and discards from the second FIFO the oldest set of sensor measurements that was in the second FIFO.

In some aspects, the measurement controller 320 may cause the sensor elements 832 to perform the measurement sequence to generate the set of sensor measurements in step 2210. In some aspects, the measurement controller 320 may store the set of sensor measurements in the memory 824 (and may discard from the memory 824 a less-recent set of sensor measurements if the memory 824 is full).

In some aspects, as shown in FIG. 22A, the process 2200 may include the step 2212 in which the analyte sensor 100 (e.g., a sensing device of the analyte sensor 100) determines whether a stop measurement command has been received. In some aspects, the stop measurement command may be conveyed by an external device (e.g., the transceiver 101 or the display device 105). In some aspects, a sensing device of the analyte sensor 100 may receive the stop measurement command via the antenna 114. In some aspects, a sensing device receiving the stop measurement command in step 2212 may include a data extractor 444 extracting data from an alternating current produced by antenna 114, a decoder of the I/O digital circuitry 334 decoding the extracted data, and/or the command decoder 322 decoding the stop measurement command from the decoded data. In some aspects, if no stop measurement command is received in step 2212, the process 2200 may proceed from step 2212 back to the step 2208. In some aspects, if a stop measurement command is received in step 2212, the process 2200 may proceed from step 2212 to a step 2214.

In some aspects, as shown in FIG. 22A, the process 2200 may include the step 2214. In some aspects, in step 2214, if a sensing device of the analyte sensor 100 has received a stop measurement command in step 2212, the sensing device (e.g., the measurement scheduler 328 of the sensing device) may stop counting the cycles of the clock 830. In some aspects, the step 2214 may include the sensing device (e.g., the measurement scheduler 328 of the sensing device) storing the count of the cycles of the clock 830 at which counting was stopped.

In some aspects, as shown in FIG. 22A, the process 2200 may include a step 2216 in which the analyte sensor 100 (e.g., a sensing device of the analyte sensor 100) receives one or more read requests. In some aspects, one or more read requests may be conveyed by an external device (e.g., the transceiver 101 or the display device 105). In some aspects, a sensing device of the analyte sensor 100 may receive the one or more read requests via the antenna 114. In some aspects, a sensing device receiving each of the one or more read requests in step 2216 may include a data extractor 444 extracting data from an alternating current produced by antenna 114, a decoder of the I/O digital circuitry 334 decoding the extracted data, and/or the command decoder 322 decoding the read request from the decoded data.

In some aspects, the step 2216 may include the sensing device conveying one or more sets of sensor measurements. In some aspects, the one or more sets of sensor measurements conveyed in step 2216 may have been stored (e.g., in the memory 824) in one or more performances of the step 2210 that occurred since the last time a start measurement command was received in step 2201. In some aspects, depending on (i) how many instances of the step 2210 have been performed since the last time a start measurement command was received in step 2201 and (ii) how many sets of sensor measurements can be stored in the memory 824, the one or more sets of sensor measurements conveyed in step 2216 may also include one or more sets of sensor measurements that were stored in the memory 824 in one or more performances of the step 2210 that occurred before the last time a start measurement command was received in step 2201. In some alternative aspects, the one or more sets of sensor measurements conveyed in step 2216 may only include the one or more sets of sensor measurements stored in one or more performances of the step 2210 that occurred since the last time a start measurement command was received in step 2201.

In some aspects, conveying one or more sets of sensor measurements may include the command decoder 322 retrieving one or more sets of sensor measurements from the memory 824, an encoder of the I/O digital circuitry 336 encoding the one or more sets of sensor measurements, and the clamp/modulator 440 of the I/O analog circuitry 336 modulating the current flowing through the antenna 114 as a function of the encoded data. In this way, the one or more sets of sensor measurements may be conveyed wirelessly by the antenna 114 as a modulated electromagnetic wave.

In some aspects, the sensing device may receive one read request from the external device, and, in response, the sensing device may iteratively select pages of the memory 824 that store sets of sensor measurements (e.g., pages MEM2 to MEM21 of FIG. 21) and convey the contents of the memory page. For example, the sensing device (e.g., the command decoder 322 of the sensing device) may select a first page of the memory 824 (e.g., MEM2), convey the one or more (e.g., two) sets of sensor measurements stored in the selected first page, select a second page of the memory 824 (e.g., MEM3), convey the one or more sets of sensor measurements stored in the selected second page, and then continue selecting pages of the memory 824 and conveying the one or more sets of sensor measurements stored therein until all of the stored sets of sensor measurements have been conveyed. In some alternative aspects, the sensing device may iteratively receive read requests for individual pages of the memory 824 that store sets of sensor measurements and then convey the one or more (e.g., two) sets of sensor measurements stored in the individual page of the memory 824 identified by the read request. For example, the sensing device may receive and decode a first read request for a first page of the memory 824 (e.g., MEM2), select the first page of the memory 824, convey the one or more (e.g., two) sets of sensor measurements stored in the selected first page, receive and decode a second read request for a second page of the memory 824 (e.g., MEM2), select the second page of the memory 824, convey the one or more (e.g., two) sets of sensor measurements stored in the selected second page, and then continue receiving and decoding read requests and conveying the one or more sets of sensor measurements until all of the stored sets of sensor measurements have been conveyed.

In some aspects, the step 2216 may include the sensing device conveying (1) the count of the cycles of the clock 830 between the sensing device (e.g., the measurement scheduler 328 of the sensing device) starting counting of the cycles of the clock 830 in step 2206 following receipt of a start measurement command and stopping counting cycles of the clock 830 in step 2214 following receipt of a stop measurement command and/or (2) a count of the cycles of the clock 830 of the sensing device since the sensing device last performed an autonomous measurement sequence in step 2210.

In some aspects, the process 2200 may proceed from step 2216 back to step 2201.

FIG. 22B is a flowchart illustrating a process 2250 that may be performed by the analyte monitoring system 50 according to some aspects. In some aspects, one or more steps of the process 2250 may be performed by an apparatus (e.g., the analyte sensor 100) of the analyte monitoring system 50. In some aspects, one or more steps of the process 2250 may be performed by a sensing device (e.g., sensing device 100A or 100B) of the analyte sensor 100. In some aspects in which the analyte sensor 100 includes two or more sensing devices, each of the sensing devices of the analyte sensor 100 may perform the process 2250. In some aspects, circuitry (e.g., mounted and/or fabricated on a substrate 112) of sensing device of the analyte sensor 100 may perform one or more steps of the process 2250.

In some aspects, as shown in FIG. 22B, the process 2250 may include a step 2203 in which the analyte sensor 100 (e.g., a sensing device of the analyte sensor 100) determines whether a command has been received. In some aspects, the command may be conveyed by an external device (e.g., the transceiver 101 or the display device 105). In some aspects, a sensing device of the analyte sensor 100 may receive the command via the antenna 114. In some aspects, a sensing device receiving a command may include a data extractor 444 extracting data from an alternating current produced by antenna 114, a decoder of the I/O digital circuitry 334 decoding extracted data, and/or a command decoder 322 decoding the command from the decoded data. In some aspects, if no command has been received in step 2203, the process 2200 may proceed to a step 2208.

In some aspects, as shown in FIG. 22B, if a command has been received in step 2203, and the command is an inventory command, the process 2250 may proceed from step 2203 to a step 2204. In some aspects, in step 2204, in response to receiving an inventory command, the sensing device of the analyte sensor 100 may convey an identification of the sensing device, which may be received by the external device. In some aspects, as shown in FIG. 22B, the process 2250 may proceed from step 2204 back to step 2203 to determine whether another command has been received.

In some aspects, as shown in FIG. 22B, if a command has been received in step 2203, and the command is a start measurement command, the process 2250 may proceed from step 2203 to a step 2206. In some aspects, in step 2206, if a sensing device of the analyte sensor 100 has received a start measurement command in step 2203, the sensing device (e.g., the measurement scheduler 328 of the sensing device) may start counting the cycles of the clock 830. In some aspects, the step 2206 may include the sensing device resetting the count of the cycles of the clock 830 (e.g., to zero) before starting to count the cycles of the clock 830. In some aspects, as shown in FIG. 22B, the process 2250 may proceed from step 2206 back to step 2203 to determine whether another command has been received. In some alternative aspects, as shown by the dashed line in FIG. 22B, the process 2250 may proceed from step 2206 to a step 2208.

In some aspects, as shown in FIG. 22B, the process 2250 may proceed to the step 2208 from step 2203 if no commands are received (and, in some aspects, from step 2206 following the start of counting clock cycles). In some aspects, in step 2208, the sensing device (e.g., the measurement scheduler 328 of the sensing device) may determine whether the sensing device (e.g., the measurement scheduler 328 of the sensing device) has counted of a threshold number of cycles of the clock 830. In some aspects, if the sensing device determines that the sensing device has not counted the threshold number of cycles of the clock 830, the process 2250 may proceed from step 2208 back to step 2203 to determine whether a command has been received. In some aspects, if the sensing device determines that the sensing device has counted the threshold number of cycles of the clock 830, the process 2250 may proceed from step 2208 to a step 2210 in which the sensing device performs a measurement sequence (e.g., an autonomous measurement sequence). In some aspects, step 2210 of the process 2250 shown in FIG. 22B may be the same as step 2210 of the process 2200 shown in FIG. 22A, which is described above. In some aspects, the process 2250 may proceed from step 2210 back to step 2203 to determine whether a command has been received.

In some aspects, as shown in FIG. 22B, if a command has been received in step 2203, and the command is a stop measurement command, the process 2250 may proceed from step 2203 to a step 2214 in which the sensing device (e.g., the measurement scheduler 328 of the sensing device) stops counting the cycles of the clock 830. In some aspects, the step 2214 may include the sensing device (e.g., the measurement scheduler 328 of the sensing device) storing the count of the cycles of the clock 830 at which counting was stopped. In some aspects, as shown in FIG. 22B, the process 2250 may proceed from step 2214 back to step 2203 to determine whether another command has been received.

In some aspects, as shown in FIG. 22B, if a command has been received in step 2203, and the command is a read request, the process 2250 may proceed from step 2203 to a step 2218 in which the sensing device conveys one or more sets of sensor measurements. In some aspects, the one or more sets of sensor measurements conveyed in step 2218 may have been stored (e.g., in the memory 824) in one or more performances of the step 2210 that occurred since the last time a start measurement command was received in step 2203. In some aspects, depending on (i) how many instances of the step 2210 have been performed since the last time a start measurement command was received in step 2203 and (ii) how many sets of sensor measurements can be stored in the memory 824, the one or more sets of sensor measurements conveyed in step 2218 may also include one or more sets of sensor measurements that were stored in the memory 824 in one or more performances of the step 2210 that occurred before the last time a start measurement command was received in step 2203. In some alternative aspects, the one or more sets of sensor measurements conveyed in step 2218 may only include the one or more sets of sensor measurements stored in one or more performances of the step 2210 that occurred since the last time a start measurement command was received in step 2203.

In some aspects, conveying one or more sets of sensor measurements may include the command decoder 322 retrieving one or more sets of sensor measurements from the memory 824, an encoder of the I/O digital circuitry 336 encoding the one or more sets of sensor measurements, and the clamp/modulator 440 of the I/O analog circuitry 336 modulating the current flowing through the antenna 114 as a function of the encoded data. In this way, the one or more sets of sensor measurements may be conveyed wirelessly by the antenna 114 as a modulated electromagnetic wave. In some aspects, after conveying the one or more sets of sensor measurements, the process 2250 may proceed from step 2218 to step 2203.

In some aspects, the sensing device may receive one read request from the external device in step 2203, and, in response, the sensing device may iteratively select pages of the memory 824 that store sets of sensor measurements (e.g., pages MEM2 to MEM21 of FIG. 21) and convey the contents of the memory page in step 2218. For example, in step 2218, the sensing device (e.g., the command decoder 322 of the sensing device) may select a first page of the memory 824 (e.g., MEM2), convey the one or more (e.g., two) sets of sensor measurements stored in the selected first page, select a second page of the memory 824 (e.g., MEM3), convey the one or more sets of sensor measurements stored in the selected second page, and then continue selecting pages of the memory 824 and conveying the one or more sets of sensor measurements stored therein until all of the stored sets of sensor measurements have been conveyed. In some alternative aspects, the sensing device may iteratively receive read requests for individual pages of the memory 824 that store sets of sensor measurements in steps 2203 and, in response to receiving each read request, convey the one or more (e.g., two) sets of sensor measurements stored in the individual page of the memory 824 identified by the read request in step 2218. For example, the sensing device may (1) receive and decode a first read request for a first page of the memory 824 (e.g., MEM2) in step 2203, (2) proceed from step 2203 to step 2218, (3) select the first page of the memory 824 and convey the one or more (e.g., two) sets of sensor measurements stored in the selected first page in step 2218, (4) proceed from step 2218 to step 2203, (5) receive and decode a second read request for a second page of the memory 824 (e.g., MEM2) in step 2203, (6) proceed from step 2203 to step 2218, (7) select the second page of the memory 824 and convey the one or more (e.g., two) sets of sensor measurements stored in the selected second page, (7) proceed from step 2218 to step 2203, and then (8) continue receiving and decoding read requests in step 2203 and conveying the one or more sets of sensor measurements in step 2218 until all of the stored sets of sensor measurements have been conveyed.

In some aspects, the step 2218 may include the sensing device conveying (1) the count of the cycles of the clock 830 between the sensing device (e.g., the measurement scheduler 328 of the sensing device) starting counting of the cycles of the clock 830 in step 2206 following receipt of a start measurement command and stopping counting cycles of the clock 830 in step 2214 following receipt of a stop measurement command and/or (2) a count of the cycles of the clock 830 of the sensing device since the sensing device last performed an autonomous measurement sequence in step 2210.

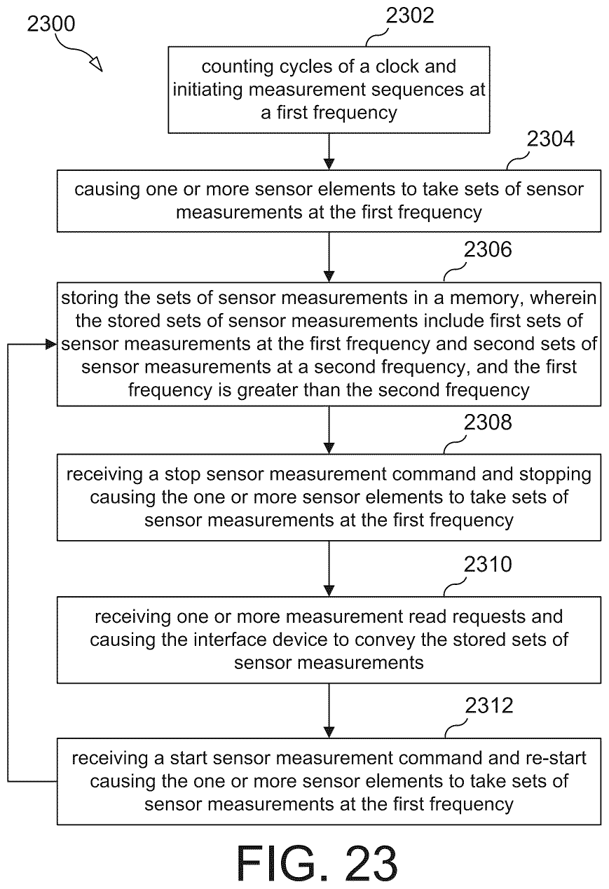

FIG. 23 is a flowchart illustrating a process 2300 that may be performed by the analyte monitoring system 50 according to some aspects. In some aspects, one or more steps of the process 2300 may be performed by an apparatus (e.g., the analyte sensor 100) of the analyte monitoring system 50. In some aspects, one or more steps of the process 2300 may be performed by a sensing device (e.g., sensing device 100A or 100B) of the analyte sensor 100. In some aspects in which the analyte sensor 100 includes two or more sensing devices, each of the sensing devices of the analyte sensor 100 may perform the process 2300. In some aspects, circuitry (e.g., mounted and/or fabricated on a substrate 112) of sensing device of the analyte sensor 100 may perform one or more steps of the process 2300.

In some aspects, as shown in FIG. 23, the process 2300 may include a step 2302 in which an apparatus (e.g. a sensing device of the analyte sensor 100) counts cycles of the clock 830 and initiates measurement sequences at a first frequency. In some aspects, the measurement scheduler 328 of the sensing device counts the cycles of the clock 830 and initiates measurement sequences at a first frequency. In some aspects, the first frequency may have a period equal to a threshold number of cycles of the clock 830. In some aspects, the step 2302 may correspond to the function performed in step 2206 and then multiple instances of step 2208 of the processes 2200 and 2250 illustrated in FIGS. 22A and 22B (e.g., following receipt of a start measurement command and before receipt of a stop measurement command).

In some aspects, as shown in FIG. 23, the process 2300 may include a step 2304 in which the apparatus (e.g, the sensing device of the analyte sensor 100) causes one or more sensor elements 832 (e.g., one or more light sources 108, 227, one or more photodetectors 224, 226, 228, 230, and/or one or more temperature transducers 464 and 492) to take sets of sensor measurements at the first frequency. In some aspects, the measurement controller 320 may be configured to, each time the measurement scheduler 328 initiates a measurement sequence, cause the one or more sensor elements 832 to take a set of sensor measurements and store the set of sensor measurements in the memory 824. In some aspects, step 2304 may correspond to the function of performed in multiple instances of step 2210 of the processes 2200 and 2250 illustrated in FIGS. 22A and 22B (e.g., following receipt of a start measurement command and before receipt of a stop measurement command).

In some aspects, the sets of sensor measurements may each include an analyte measurement based on the detectable property of the analyte indicator of the indicator element. In some aspects, the detectable property of the analyte indicator is a first detectable property, the first detectable property additionally varies in accordance with an effect on the analyte indicator, the indicator element further includes an interferent indicator having a second detectable property that varies in accordance with the effect on the analyte indicator, and the sets of sensor measurements each include an interferent measurement based on the second detectable property. In some aspects, the sets of sensor measurements may additionally or alternatively each include a temperature measurement.

In some aspects, as shown in FIG. 23, the process 2300 may include a step 2306 in which the apparatus (e.g, the sensing device of the analyte sensor 100) stores the sets of sensor measurements in the memory 824. In some aspects, the stored sets of sensor measurements may include first sets of sensor measurements at the first frequency and second sets of sensor measurements at a second frequency. In some aspects, the first frequency may be greater than the second frequency. In some aspects, the first sets of sensor measurements at the first frequency may be more recent sets of sensor measurements than the second sets of sensor measurements at the second frequency. In some aspects, the step 2306 may include the apparatus (e.g, the sensing device of the analyte sensor 100) storing in the memory 824, for each of the stored sets of sensor measurements, a count of the cycles of the clock 830 at the time the set of sensor measurements was taken. In some aspects, step 2306 may correspond to the function of performed in multiple instances of step 2210 of the process 2200 and 2250 illustrated in FIGS. 22A and 22B (e.g., following receipt of a start measurement command and before receipt of a stop measurement command).

In some aspects, storing the sets of sensor measurements in the memory 824 in step 2306 may include down-sampling previously-stored sets of sensor measurements. In some aspects, at some times, down-sampling previously-stored sets of sensor measurements may include discarding a previously-stored set of sensor measurements that is not an oldest set of sensor measurements. In some aspects, at other times, down-sampling previously-stored sets of sensor measurements may include discarding a previously-stored set of sensor measurements that is an oldest set of sensor measurements.

In some aspects, as shown in FIG. 23, the process 2300 may include a step 2308 in which the apparatus (e.g, the sensing device of the analyte sensor 100) receives a stop sensor measurement command using an interface device (e.g., antenna 114 of the analyte sensor 100). In some aspects, the step 2308 may include, if the stop sensor measurement command is received, stop causing the one or more sensor elements 832 to take sets of sensor measurements at the first frequency. In some aspects, step 2310 may correspond to steps 2212 and 2214 of the process 2200 illustrated in FIG. 22A or step 2214 of the process 2250 illustrated in FIG. 22B (e.g., following receipt of a stop measurement command).

In some aspects, as shown in FIG. 23, the process 2300 may include a step 2310 in which the apparatus (e.g, the sensing device of the analyte sensor 100) receives one or more measurement read requests using the interface device (e.g., antenna 114 of the analyte sensor 100). In some aspects, the step 2310 may include, if the one or more measurement read requests are received, causing the interface device to convey the stored sets of sensor measurements. In some aspects, the step 2310 may include, if the one or more measurement read requests are received, causing the interface device (e.g., antenna 114 of the analyte sensor 100) to convey with the stored sets of sensor measurements a count of the cycles of the clock 830. In some aspects, the apparatus may cause the interface device to convey the stored sets of sensor measurements in step 2310 while the apparatus (e.g., sensing device of the analyte sensor 100) is stopped from causing the one or more sensor elements 832 to take sets of sensor measurements at the first frequency. In some aspects, step 2310 may correspond to step 2216 of the process 2200 illustrated in FIG. 22A or steps 2203 and 2218 of the process 2250 illustrated in FIG. 22B (e.g., in which one or more read requests are received in step 2203).

In some aspects, as shown in FIG. 23, the process 2300 may include a step 2312 in which the apparatus (e.g, the sensing device of the analyte sensor 100) receives a start sensor measurement command using the interface device (e.g., antenna 114 of the analyte sensor 100). In some aspects, the step 2310 may include, if a start sensor measurement command is received, re-start causing the one or more sensor elements 832 to take sets of sensor measurements at the first frequency. In some aspects, step 2312 may correspond to step 2206 and multiple instances of steps 2208 and 2210 of the processes 2200 and 2250 illustrated in FIGS. 22A and 22B (e.g., following receipt of the start measurement command and before receipt of a stop measurement command).

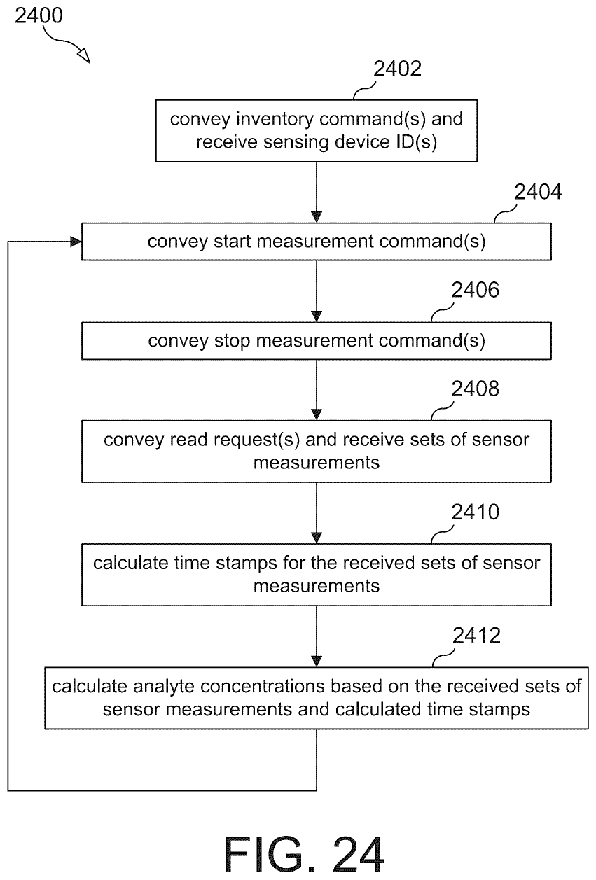

FIG. 24 is a flowchart illustrating a process 2400 that may be performed by the analyte monitoring system 50 according to some aspects. In some aspects, one or more steps of the process 2400 may be performed by an apparatus (e.g., the transceiver 101, the display device 105, and/or DMS 121) of the analyte monitoring system 50. In some aspects, one or more steps of the process 2400 may be performed by a computer of an apparatus (e.g., the PIC controller 920 of the transceiver 101, the computer 310 of the display device 105, and/or a computer of the DMS 121) of the analyte monitoring system 50.

In some aspects, as shown in FIG. 24, the process 2400 may include a step 2402 in which the apparatus (e.g., the transceiver 101, the display device 105, and/or DMS 121) conveys one or more inventory commands. In some aspects, a computer (e.g., the PIC controller 920 of the transceiver 101, the computer 310 of the display device 105, or a computer of the DMS 121) of the apparatus may use an interface device (e.g., the third wireless communication IC 317 and/or antenna) of the display device 105 to convey the one or more inventory commands. In some aspects, an inventory command conveyed by the apparatus in step 2402 may be received by the sensing device(s) of the analyte sensor 100 (e.g., in step 2201 of the process 2200 shown in FIG. 22A or step 2203 of the process 2250 shown in FIG. 22B).

In some aspects, the step 2402 may include the apparatus (e.g., the transceiver 101, the display device 105, and/or DMS 121) receiving an identification of the sensing device(s) of the analyte sensor 100, which may be conveyed by the sensing device(s) of the analyte sensor 100 (e.g., in step 2204 of the processes 2200 and 2250 shown in FIGS. 22A and 22B). In some aspects in which the analyte sensor 100 includes first and second sensing devices 100A and 100B, the step 2402 may include the apparatus receiving an identification of the first sensing device 100A, which was conveyed by the first sensing device 100A, and an identification of the second sensing device 100B, which was conveyed by the second sensing device 100B.

In some aspects, in step 2402, the apparatus (e.g., the transceiver 101, the display device 105, and/or the DMS 121) may convey a single unaddressed inventory command to all of the sensing devices of the analyte sensor 100, and each of the sensing devices (e.g., first and second sensing devices 100A and 100B) of the analyte sensor 100 may convey identifications in response to the single unaddressed inventory command. In some alternative aspects, in step 2402, the apparatus may convey, for each of the sensing devices of the analyte sensor 100, an inventory command addressed to the sensing device. For example, in some alternative aspects in which the analyte sensor 100 includes first and second sensing devices 100A and 100B, the apparatus may convey an inventory command addressed to the first sensing device 100A and an inventory command addressed to the second sensing device 100B. In this example, the first sensing device 100A may convey identification information in response to the inventory command addressed to the first sensing device 100A, and the second sensing device 100B may convey identification information in response to the inventory command addressed to the second sensing device 100B.

In some aspects, as shown in FIG. 24, the process 2400 may include a step 2404 in which the apparatus (e.g., the transceiver 101, the display device 105, and/or the DMS 121) conveys one or more start measurement commands. In some aspects, a computer (e.g., the PIC controller 920 of the transceiver 101, the computer 310 of the display device 105, or a computer of the DMS 121) of the apparatus may use an interface device (e.g., the third wireless communication IC 317 and/or antenna) of the display device 105 to convey the one or more start measurement commands. In some aspects, the one or more start measurement commands conveyed by the apparatus may be received by the sensing device(s) of the analyte sensor 100 (e.g., in step 2201 of the process 2200 shown in FIG. 22A, step 2203 of the process 2250 shown in FIG. 22B, and/or step 2312 of the process 2300 shown in FIG. 23). In some aspects, the one or more start measurement commands conveyed by the apparatus may cause each of the one or more sensing devices (e.g., each of the first and second sensing devices 100A and 100B) of the analyte sensor 100 to take and store sets of sensor measurements at a first frequency that has a period equal to a threshold number of cycles of the clock 830 of the sensing device of the analyte sensor 100 (e.g., in step 2210 of the processes 2200 and 2250 shown in FIGS. 22A and 22B and/or steps 2304 and 2306 of the process 2300 shown in FIG. 23).

In some aspects, in step 2404, the apparatus (e.g., the transceiver 101, the display device 105, and/or the DMS 121) may convey a single unaddressed start measurement command to all the sensing devices of the analyte sensor 100, and each of the sensing devices of the analyte sensor may act in response to the unaddressed start measurement command. In some alternative aspects, in step 2404, the apparatus may convey, for each of the sensing devices of the analyte sensor 100, a start measurement command addressed to the sensing device. For example, in some alternative aspects in which the analyte sensor 100 includes first and second sensing devices 100A and 100B, the apparatus may convey a start measurement command addressed to the first sensing device 100A and a start measurement command addressed to the second sensing device 100B.

In some aspects, as shown in FIG. 24, the process 2400 may include a step 2406 in which the apparatus (e.g., the transceiver 101, the display device 105, and/or DMS 121) conveys a stop measurement command. In some aspects, a computer (e.g., the PIC controller 920 of the transceiver 101, the computer 310 of the display device 105, or a computer of the DMS 121) of the apparatus may use an interface device (e.g., the third wireless communication IC 317 and/or antenna) of the display device 105 to convey the stop measurement command. In some aspects, the stop measurement command conveyed by the apparatus may be received by the sensing device(s) of the analyte sensor 100 (e.g., in step 2212 of the process 2200 shown in FIG. 22A, step 2203 of the process 2250 shown in FIG. 22B, and/or step 2308 of the process 2300 shown in FIG. 23).

In some aspects, in step 2406, the apparatus (e.g., the transceiver 101, the display device 105, and/or the DMS 121) may convey a single unaddressed stop measurement command to all the sensing devices of the analyte sensor 100, and each of the sensing devices of the analyte sensor may act in response to the unaddressed stop measurement command. In some alternative aspects, in step 2406, the apparatus may convey, for each of the sensing devices of the analyte sensor 100, a stop measurement command addressed to the sensing device. For example, in some alternative aspects in which the analyte sensor 100 includes first and second sensing devices 100A and 100B, the apparatus may convey a stop measurement command addressed to the first sensing device 100A and a stop measurement command addressed to the second sensing device 100B.

In some aspects, as shown in FIG. 24, the process 2400 may include a step 2408 in which the apparatus (e.g., the transceiver 101, the display device 105, and/or DMS 121) conveys one or more read requests. In some aspects, a computer (e.g., the PIC controller 920 of the transceiver 101, the computer 310 of the display device 105, or a computer of the DMS 121) of the apparatus may use an interface device (e.g., the third wireless communication IC 317 and/or antenna) of the display device 105 to convey the one or more read requests. In some aspects, the one or more read requests conveyed by the apparatus may be received by the sensing device(s) of the analyte sensor 100 (e.g., in step 2216 of the process 2200 shown in FIG. 22A, step 2203 of the process 2250 shown in FIG. 22B, and/or step 2310 of the process 2300 shown in FIG. 23). In some aspects, the step 2408 may include the apparatus receiving one or more sets of sensor measurements conveyed by the sensing device(s) of the analyte sensor 100.

In some aspects, in step 2408, the apparatus may convey one addressed read request to each sensing device of the analyte sensor 100, and, in response, the sensing device to which the read request is addressed may iteratively select pages of the memory 824 that store sets of sensor measurements (e.g., pages MEM2 to MEM21 of FIG. 21) and convey the contents of the memory page, which may be received by the apparatus. In some alternative aspects, in step 2408, for each sensing device, the apparatus may iteratively convey read requests for individual pages of the memory 824 of the sensing device that store sets of sensor measurements, and, in response, the sensing device to which the read request is addressed may convey the one or more (e.g., two) sets of sensor measurements stored in the individual page of the memory 824 of the sensing device identified by the read request, which may be received by the apparatus. In some further alternative aspects, in step 2408, the apparatus may convey one or more unaddressed read requests, and, in response, each of the sensing devices may convey sets of sensor measurements, which may be received by the apparatus.

In some aspects, the step 2408 may include the apparatus receiving, from one or more of the sensing devices of the analyte sensor 100, (1) a count of the cycles of the clock 830 of the sensing device between the sensing device (e.g., the measurement scheduler 328 of the sensing device) starting counting of the cycles of the clock 830 (e.g., in step 2206 of the processes 2200 and 2250 shown in FIGS. 22A and 22B) following receipt of a start measurement command and stopping counting cycles of the clock 830 (e.g., in step 2214 of the processes 2200 and 2250 shown in FIGS. 22A and 22B) following receipt of a stop measurement command and/or (2) a count of the cycles of the clock 830 of the sensing device since the sensing device last performed an autonomous measurement sequence (e.g., in step 2210 of the processes 2200 and 2250 shown in FIGS. 22A and 22B).

In some aspects, as shown in FIG. 24, the process 2400 may include a step 2410 in which the apparatus (e.g., the transceiver 101, the display device 105, and/or DMS 121) calculates time stamps for the received sets of sensor measurements. In some aspects, a computer (e.g., the PIC controller 920 of the transceiver 101, the computer 310 of the display device 105, or a computer of the DMS 121) of the apparatus may calculate time stamps for the received sets of sensor measurements.

In some aspects, each of the sets of sensor measurements may include timing information, and the apparatus (e.g., a computer of the apparatus) may be configured to use at least the timing information of the sets of sensor measurements to calculate the time stamps for the sets of sensor measurements. In some aspects, the timing information of a set of sensor measurements may include a count of the cycles of the clock 830 (e.g., as counted by a measurement scheduler 328 of a sensing device of the analyte sensor 100 starting in step 2206 of the processes 2200 and 2250 shown in FIGS. 22A and 22B) at the time the set of sensor measurements was taken (e.g., by the sensing device of the analyte sensor 100). In some aspects, the count of the cycles of the clock 830 at the time the set of sensor measurements was taken may be the count of the cycles of the clock 830 when the sensing device (e.g., the measurement scheduler 328 of the sensing device) determines that the sensing device has counted of a threshold number of cycles of the clock 830 in step 2208 of the processes 2200 and 2250 shown in FIGS. 22A and 22B. In some aspects, the timing information may additionally or alternatively include a number n for the autonomous sensor measurement (e.g., indicating that the autonomous sensor measurement is the nth autonomous sensor measurement that the sensing device of the analyte sensor 100 took since autonomous measurements were started). In some aspects, the number n may be indicative of the number of times the sensing device (e.g., the measurement scheduler 328 of the sensing device) determines that the sensing device has counted of a threshold number of cycles of the clock 830 in step 2208 of the processes 2200 and 2250 shown in FIGS. 22A and 22B.

In some aspects, each of the sets of sensor measurements may include a temperature measurement (e.g., a measurement by a temperature transducer 464 or 492 of a temperature of a sensing device of the analyte sensor 100), and the apparatus (e.g., a computer of the apparatus) may be configured to use at least the timing information and one or more of the temperature measurements of the sets of sensor measurements to calculate the time stamps for the sets of sensor measurements in step 2410. In some aspects, the apparatus (e.g., a computer of the apparatus) may be configured to use at least the timing information and the one or more of the temperature measurements of the sets of sensor measurements and a characterization of a temperature dependence of the cycles of the clock 830 of a sensing device of the analyte sensor 100 to calculate the time stamps for the sets of sensor measurements in step 2410. In some aspects, the apparatus (e.g., a computer of the apparatus) may be configured to use at least the timing information and the one or more of the temperature measurements of the sets of sensor measurements, the characterization of the temperature dependence of the cycles of the clock 830 of the analyte sensor 100, and one or both of a time at which the apparatus conveyed one or more start sensor measurement commands in step 2404 and a time at which the apparatus conveyed one or more stop measurement commands to calculate the time stamps for the sets of sensor measurements in step 2410. In some aspects, the apparatus (e.g., a computer of the apparatus) may be configured to use at least the timing information and the one or more of the temperature measurements of the sets of sensor measurements, the characterization of the temperature dependence of the cycles of the clock 830 of the analyte sensor 100, and the first frequency to calculate the time stamps for the sets of sensor measurements.

In some aspects, each of the sets of sensor measurements may include a voltage measurement (e.g., a measurement of the voltage VBAT produced by the CSD 202), and the apparatus (e.g., a computer of the apparatus) may be configured to use at least the timing information and one or more of the voltage measurements of the sets of sensor measurements to calculate the time stamps for the sets of sensor measurements in step 2410. In some aspects, the apparatus (e.g., a computer of the apparatus) may be configured to use at least the timing information and the one or more of the voltage measurements of the sets of sensor measurements and a characterization of a voltage dependence of the cycles of the clock 830 of a sensing device of the analyte sensor 100 to calculate the time stamps for the sets of sensor measurements in step 2410. In some aspects, the apparatus (e.g., a computer of the apparatus) may be configured to use at least the timing information and the one or more of the voltage measurements of the sets of sensor measurements, the characterization of the voltage dependence of the cycles of the clock 830 of the analyte sensor 100, and one or both of a time at which the apparatus conveyed one or more start sensor measurement commands in step 2404 and a time at which the apparatus conveyed one or more stop measurement commands to calculate the time stamps for the sets of sensor measurements in step 2410. In some aspects, the apparatus (e.g., a computer of the apparatus) may be configured to use at least the timing information and the one or more of the voltage measurements of the sets of sensor measurements, the characterization of the voltage dependence of the cycles of the clock 830 of the analyte sensor 100, and the first frequency to calculate the time stamps for the sets of sensor measurements.

In some aspects, each of the sets of sensor measurements may include a temperature measurement and a voltage measurement, and the apparatus (e.g., a computer of the apparatus) may be configured to use at least the timing information, one or more of the temperature measurements, and one or more of the voltage measurements of the sets of sensor measurements to calculate the time stamps for the sets of sensor measurements in step 2410. In some aspects, the apparatus (e.g., a computer of the apparatus) may be configured to use at least the timing information, the one or more of the temperature measurements, and the one or more of the voltage measurements of the sets of sensor measurements, and characterizations of a temperature dependence and a voltage dependence of the cycles of the clock 830 of a sensing device of the analyte sensor 100 to calculate the time stamps for the sets of sensor measurements in step 2410. In some aspects, the apparatus (e.g., a computer of the apparatus) may be configured to use at least the timing information, the one or more of the temperature measurements, and the one or more of the voltage measurements of the sets of sensor measurements, the characterizations of the temperature dependence and the voltage dependence of the cycles of the clock 830 of the analyte sensor 100, and one or both of a time at which the apparatus conveyed one or more start sensor measurement commands in step 2404 and a time at which the apparatus conveyed one or more stop measurement commands to calculate the time stamps for the sets of sensor measurements in step 2410. In some aspects, the apparatus (e.g., a computer of the apparatus) may be configured to use at least the timing information, the one or more of the temperature measurements, and the one or more of the voltage measurements of the sets of sensor measurements, the characterization of the temperature dependence and the voltage dependence of the cycles of the clock 830 of the analyte sensor 100, and the first frequency to calculate the time stamps for the sets of sensor measurements.

In some aspects, the sets of sensor measurements received in step 2408 may include first sets of sensor measurements, which were stored by the analyte sensor 100 (e.g., by a sensing device of the analyte sensor 100) at the first frequency, and second sets of sensor measurements, which were stored by the analyte sensor 100 at a second frequency that is less than the first frequency. In some aspects, the apparatus (e.g., a computer of the apparatus) may be configured to, in calculating the time stamps for the sets of sensor measurements in step 2410, calculate time stamps for the first sets of sensor measurements and calculate time stamps for the second sets of sensor measurements. In some aspects, the first sets of sensor measurements may be more recent sets of sensor measurements than the second sets of sensor measurements.

In some aspects, the frequency and period of the clock 830 of the analyte sensor 100 may be stable (e.g., not affected by temperature and voltage changes), or the apparatus (e.g., a computer of the apparatus) may treat the frequency and period of the clock 830 of the analyte sensor 100 as stable by not correcting for changes to the frequency and period of the clock 830 of the analyte sensor 100 due to changes in temperature and/or voltage. In some aspects in which the apparatus (e.g., a computer of the apparatus) does not correct for changes to the frequency and period of the clock 830 of the analyte sensor 100 due to changes in temperature and/or voltage when calculating time stamps for the sets of sensor measurements in step 2410, the apparatus (e.g., a computer of the apparatus) may calculate a time stamp for the nth set of sensor measurements using the following equation:

$$Automeas\_time ( n ) = time\_start + time\_elapsed ( n ) \qquad ( 1 )$$

where time_start is the time at which the apparatus (e.g., the transceiver 101 or the display device 105) conveyed the one or more start sensor measurement commands in step 2404, and the time_elapsed (n) is a calculated amount of time that has elapsed between the time at which the one or more start sensor measurement commands were conveyed in step 2404 (i.e., time_start) and the time at which the analyte sensor 100 performed a measurement sequence to take the nth set of sensor measurements (e.g., in step 2210 of FIG. 22A or 22B or step 2304 or 2312 of FIG. 23) following the conveyance of the one or more start sensor measurement commands. In some aspects, the apparatus (e.g., a computer of the apparatus) may calculate time_elapsed (n) using the following equation:

$$time\_elapsed ( n ) = automeas\_time ( n ) \times rtc\_ref / RTC\_freq \qquad ( 2 )$$

where automeas_number (n) is the nth measurement of the set of sensor measurements, rtc_ref is the number of pulses or cycles of the clock 830 of the analyte sensor 100 that the sensing device of the analyte sensor 100 is programmed to allow to occur between successive measurement sequences, and RTC_freq is the frequency of the clock 830 of the analyte sensor 100.

In some aspects in which the apparatus (e.g., a computer of the apparatus) corrects for changes to the frequency and period of the clock 830 of the analyte sensor 100 due to at least changes in temperature when calculating time stamps for the sets of sensor measurements in step 2410, a calibration may be performed (e.g., during the manufacturing process) to measure the frequency and/or period of the clock 830 of the analyte sensor 100 at different temperatures. For example, in some aspects, the frequency of the clock 830 may be measured at 37° C., 15° C., and 50° C. as RTC_freq_37C, RTC_freq_15C, and RTC_freq_50C, respectively. In some aspects, the measurements of the frequency and/or period of the clock 830 of the analyte sensor 100 at different temperatures (e.g., RTC_freq_37C, RTC_freq_15C, and RTC_freq_50C) may be used to determine one or more coefficients of temperature dependence (e.g., cfreqT, cfreqT1 and cfreqT2, cperiodT, or cperiodT1 and cperiodT2). In some aspects, in step 2410, the apparatus (e.g., a computer of the apparatus) may use the one or more coefficients of temperature dependence to calculate a temperature dependent frequency of the clock 830 (RTC_freq (T)). In some aspects, the apparatus (e.g., a computer of the apparatus) may calculate the temperature dependent frequency of the clock 830 using one of the following equations:

$$RTC\_freq ( T ) = RTCfreq\_ 37 \times ( 1 + cfreqT * ( T - 37 ) ) \qquad ( 3 )$$

$$RTC\_freq ( T ) = RTCfreq\_ 37 \times ( 1 + {cfreqT}_{1} * ( T - 37 ) + {cfreqT}_{2} * {( T - 37 )}^{2} ) \qquad ( 4 )$$

$$RTC\_freq ( T ) = RTCfreq\_ 37 / ( 1 + cperiodT * ( T - 37 ) ) \qquad ( 5 )$$

$$RTC\_freq ( T ) = RTCfreq\_ 37 / ( 1 + {cperiodT}_{1} + ( T - 37 ) + {cperiodT}_{2} * {( T - 37 )}^{2} ) \qquad ( 6 )$$

where T is the temperature of the sensing device of the analyte sensor 100 (e.g., as measured by the temperature transducer 464 and/or 492). In some aspects in which the apparatus (e.g., a computer of the apparatus) corrects for changes to the frequency and period of the clock 830 of the analyte sensor 100 due to at least changes in temperature when calculating time stamps for the sets of sensor measurements in step 2410, the apparatus (e.g., a computer of the apparatus) may calculate a time stamp for the nth set of sensor measurements using the following equation:

$$Automeas\_time ( n , T ) = time\_start + time\_elapsed ( n , T ) \qquad ( 7 )$$

where time_elapsed (n,T) is calculated using the following equation:

$$time\_elapsed ( n , T ) = automeas\_number ( n ) \times rtc\_ref / RTC\_freq ( T ) \qquad ( 8 )$$

In some aspects, temperature correction for changes to the frequency of the clock 830 due to changes in temperature in this manner when calculating time stamps may work particularly well when the temperature T is stable.

In some alternative aspects, the temperature correction for changes to the frequency of the clock 830 due to changes in temperature may account for temperature and frequency changes at each set of sensor measurements. In some aspects, in step 2410, the apparatus (e.g., a computer of the apparatus) may calculate a time stamp for the nth set of sensor measurements that accounts for different frequencies due to different temperatures at each set of sensor measurements using the following equation:

$$Automeas\_time [ n ] = time\_start + {\sum }_{m = 1}^{n} rtc\_ref / RTC\_freq [ T [ m ] ] \qquad ( 9 )$$

In some aspects, in step 2410, for the 2^{nd} and greater sets of sensor measurements (e.g., n>1), the apparatus (e.g., a computer of the apparatus) may calculate a time stamp for the nth set of sensor measurements that considers the average temperature during the time between the nth and n-1^{th} sets of sensor measurements using the following equation:

$$Automeas\_time [ n ] = timestart + rtc\_ref / RTC\_freq [ T [ m ] ] + {\sum }_{m = 2}^{n} rtc\_ref / RTC\_freq [ \frac{T [ m ] + T [ m - 1 ]}{2} ] \qquad ( 10 )$$

In some alternative aspects, in step 2410, for the 2^{nd} and greater sets of sensor measurements (e.g., n>1), the apparatus (e.g., a computer of the apparatus) may calculate a time stamp for the nth set of sensor measurements that considers the average frequency during the time between the nth and n-1^{th} sets of sensor measurements using the following equation:

$$Automeasurementtime [ n ] = timestart + rtc\_ref / RTC\_freq [ T [ n ] ] + {\sum }_{m = 2}^{n} rtc\_ref / [ \frac{RTC\_freq [ T [ m ] ] + RTC\_freq [ T [ m - 1 ] ]}{2} ] \qquad ( 11 )$$

In some alternative aspects, in step 2410, for the 2^{nd} and greater sets of sensor measurements (e.g., n>1), the apparatus (e.g., a computer of the apparatus) may calculate a time stamp for the nth set of sensor measurements that considers the average period during the time between the nth and n-1^{th} sets of sensor measurements using the following equation:

$$Automeasurement\_time [ n ] = time\_start + {\sum }_{m = 1}^{n} ( rtc\_ref / RTC\_freq [ T [ m ] ] + rtc\_ref / RTC\_freq [ T [ m - 1 ] ] ) / 2 \qquad ( 12 )$$

In some aspects in which the apparatus (e.g., a computer of the apparatus) corrects for changes to the frequency and period of the clock 830 of the analyte sensor 100 due to at least changes in temperature and voltage when calculating time stamps for the sets of sensor measurements in step 2410, a calibration may be performed (e.g., during the manufacturing process) to measure the frequency and/or period of the clock 830 of the analyte sensor 100 at different voltages produced by the charge storage device 202 of the analyte sensor 100. For example, in some aspects, the frequency of the clock 830 may be measured at voltages produced by the charge storage device 202 of 2.6V, 2.8V, 3.0V, and 3.2V (and with the sensing device of the analyte sensor 100 at a constant temperature such as, for example, 37° C.) as RTC_freq_2p6V, RTC_freq_2p8V, RTC_freq_3p0V, and RTC_freq_3p2V, respectively. In some aspects, the measurements of the frequency and/or period of the clock 830 of the analyte sensor 100 at different voltages (e.g., RTC_freq_2p6V, RTC_freq_2p8V, RTC_freq_3p0V, and RTC_freq_3p2V) may be used to determine one or more coefficients of voltage dependence (e.g., cfreqV, cfreqV1 and cfreqV2, cperiodV, or cperiodV1 and cperiodV2). In some aspects, in step 2410, the apparatus (e.g., a computer of the apparatus) may use the one or more coefficients of voltage dependence to calculate a temperature and voltage dependent frequency of the clock 830 (RTC_freq (T,V)). In some aspects, the apparatus (e.g., a computer of the apparatus) may calculate the temperature and voltage dependent frequency of the clock 830 using one of the following equations:

$$RTC\_freq ( T , V ) = RTCfreq ( T ) \times ( 1 + cfreqV \times ( 2.8 - V ) ) \qquad ( 13 )$$

$$RTC\_freq ( T , V ) = RTCfreq ( T ) \times ( 1 + {cfreqV}_{1} \times ( 2.7 - V ) + {cfreqV}_{2} \times {( 2.7 - V )}^{2} ) \qquad ( 14 )$$

$$RTC\_freq ( T , V ) = RTCfreq ( T ) / ( 1 + cfreqV \times ( 2.8 - V ) ) \qquad ( 15 )$$

$$RTC\_freq ( T , V ) = RTCfreq ( T ) / ( 1 + {cperiodV}_{1} \times ( 2.7 - V ) + {cperiodV}_{2} \times {( 2.7 - V )}^{2} ) \qquad ( 16 )$$

$$RTC\_freq ( T , V ) = RTCfreq ( T ) + piecewise linear voltage dependence \qquad ( 17 )$$

where V a measurement of the voltage VBAT produced by the charge storage device 202, which may be generated by the CSD monitor 466 and digitized by the ADC 482. In some aspects in which the apparatus (e.g., a computer of the apparatus) corrects for changes to the frequency and period of the clock 830 of the analyte sensor 100 due to at least changes in temperature and voltage when calculating time stamps for the sets of sensor measurements in step 2410, the apparatus (e.g., a computer of the apparatus) may calculate a time stamp for the nth set of sensor measurements using the following equation:

$$Automeas\_time ( n , T , V ) = time\_start + time\_elapsed ( n , T , V ) \qquad ( 18 )$$

where time_elapsed (n,T.V) is calculated using the following equation:

$$time\_elapsed ( n , T , V ) = automeas\_number ( n ) \times rtc\_ref / RTC\_freq ( T , V ) \qquad ( 19 )$$

In some aspects, temperature and voltage correction for changes to the frequency of the clock 830 due to changes in temperature and voltage in this manner when calculating time stamps may work particularly well when the temperature T and voltage V are stable.

In some alternative aspects, the voltage correction for changes to the frequency of the clock 830 due to changes in voltage may account for voltage (and therefore frequency) changes at each set of sensor measurements. In some aspects, in step 2410, the apparatus (e.g., a computer of the apparatus) may calculate a time stamp for the nth set of sensor measurements that accounts for different frequencies due to different voltages at each set of sensor measurements using the following equation:

$$Automeas\_time [ n ] = time\_start + {\sum }_{m = 1}^{n} rtc\_ref / RTC\_freq [ V [ m ] ] \qquad ( 20 )$$

In some aspects, in step 2410, for the 2^{nd} and greater sets of sensor measurements (e.g., n>1), the apparatus (e.g., a computer of the apparatus) may calculate a time stamp for the nth set of sensor measurements that considers the average voltage during the time between the nth and n-1^{th} sets of sensor measurements using the following equation:

$$Automeas\_time [ n ] = timestart + rtc\_ref / RTC\_freq [ V [ n ] ] + {\sum }_{m = 2}^{n} rtc\_ref / RTC\_freq [ \frac{V [ m ] + V [ m - 1 ]}{2} ] \qquad ( 21 )$$

In some alternative aspects, in step 2410, for the 2^{nd} and greater sets of sensor measurements (e.g., n>1), the apparatus (e.g., a computer of the apparatus) may calculate a time stamp for the nth set of sensor measurements that considers the average frequency during the time between the nth and n-1^{th} sets of sensor measurements using the following equation:

$$Automeas\_time [ n ] = timestart + rtc\_ref / RTC\_freq [ V [ n ] ] + {\sum }_{m = 2}^{n} rtc\_ref / ( \frac{RTC\_freq [ V [ m ] ] + RTC\_freq [ V [ m - 1 ] ]}{2} ) \qquad ( 22 )$$

In some alternative aspects, in step 2410, for the 2^{nd} and greater sets of sensor measurements (e.g., n>1), the apparatus (e.g., a computer of the apparatus) may calculate a time stamp for the nth set of sensor measurements that considers the average period during the time between the nth and n-1^{th} sets of sensor measurements using the following equation:

$$Automeas\_time [ n ] = time\_start + {\sum }_{m = 1}^{n} ( rtc\_ref / RTC\_freq [ V [ n ] ] + rtc\_ ref / RTC\_freq [ V [ m - 1 ] ] ) / 2 \qquad ( 23 )$$

In some alternative aspects, the voltage correction for changes to the frequency of the clock 830 due to changes in voltage may account for temperature and voltage (and therefore frequency) changes at each set of sensor measurements. In some aspects, in step 2410, the apparatus (e.g., a computer of the apparatus) may calculate a time stamp for the nth set of sensor measurements that accounts for different frequencies due to different frequencies and different voltages at each set of sensor measurements using the following equation:

$$Automeas\_time [ n ] = time\_start + {\sum }_{m = 1}^{n} rtc\_ref / RTC\_freq [ V [ m ] , T [ m ] ] \qquad ( 24 )$$

In some aspects, in step 2410, for the 2^{nd} and greater sets of sensor measurements (e.g., n>1), the apparatus (e.g., a computer of the apparatus) may calculate a time stamp for the nth set of sensor measurements that considers the average frequency during the time between the nth and n-1^{th} sets of sensor measurements using the following equation:

$$Automeas\_time [ n ] = timestart + rtc\_ref / RTC\_freq [ V [ n ] ] + {\sum }_{m = 2}^{n} rtc\_ref / ( \frac{RTC\_freq [ V [ m ] , T [ m ] ] + RTC\_freq [ V [ m - 1 ] , T [ m - 1 ] ]}{2} ) \qquad ( 25 )$$

In some alternative aspects, in step 2410, for the 2^{nd} and greater sets of sensor measurements (e.g., n>1), the apparatus (e.g., a computer of the apparatus) may calculate a time stamp for the nth set of sensor measurements that considers the average period during the time between the nth and n-1^{th} sets of sensor measurements using the following equation:

$$Automeasurement\_time [ n ] = time\_start + {\sum }_{m = 1}^{n} ( rtc\_ref / RTC\_freq [ V [ m ] , T [ m ] ] + rtc\_ref / RTC\_freq [ V [ m - 1 ] , T [ m - 1 ] ] ) / 2 \qquad ( 26 )$$

In some aspects, as described above, the apparatus may calculate the time stamps in step 2410 using a forward calculation that adds the time elapsed (n) to the time_start. However, this is not required. In some alternative aspects, the apparatus (e.g., a computer of the apparatus) may use a backward calculation that calculates a time stamp for the nth set of sensor measurements using the following equation:

$$Automeas\_time ( n ) = time\_last ( T , V ) - time\_since ( n , T , V ) \qquad ( 27 )$$

where time_last (T,V) is a calculated time at which the analyte sensor 100 took the last set of sensor measurements before the one or more stop measurement commands were convted in step 2406. In some aspects, time_last (T,V) may be calculated using the equation below:

$$time\_last ( T , V ) = time\_stopped - time\_since \_last ( T , V ) \qquad ( 28 )$$

where time_stopped is the time at which the apparatus (e.g., the transceiver 101 or the display device 105) conveyed the one or more stop sensor measurement commands in step 2406, and time_since_last (T,V) is a calculated amount of time that elapsed between the time at which the last set of sensor measurements was taken and the time at which the apparatus conveyed the one or more stop sensor measurement commands in step 2406. In some aspects, time_since_last (T,V) may be calculated using the following equation:

$$time\_since \_last ( T , V ) = last\_rtc \_value / RTC\_freq ( T , V ) \qquad ( 29 )$$

where last_rtc_value is a count of the cycles of the clock 830 of the sensing device of the analyte sensor 100 since the sensing device performed the autonomous measurement sequence to take last set of sensor measurements (e.g., in step 2210 of the processes 2200 and 2250 shown in FIGS. 22A and 22B), which the apparatus may receive from the analyte sensor 100. In some aspects, the time_since (n,T,V) may be a calculated amount of time that has elapsed between the time at which the analyte sensor 100 performed a measurement sequence to take the nth set of sensor measurements (e.g., in step 2210 of FIG. 22A or 22B or step 2304 or 2312 of FIG. 23) and the time at which the analyte sensor 100 performed a measurement sequence to take the last set of sensor measurements. In some aspects, time_since (n,T,V) may be calculated using the following equation:

$$time\_since ( n , T , V ) = ( last\_automeas \_number - automeas\_number ( n ) ) / RTC\_freq ( T , V ) \qquad ( 30 )$$

where automeas_number (n) egual is the nth measurement, and last_automeas_number is the number of the last set of sensor measurements that occurred before the autonomous measurements were stopped by the one or more stop sensor measurement commands. For instance, if a sensing device of the analyte sensor 100 performed 31 measurement sequences to take 31 sets of sensor measurements between receiving a start measurement command and then receiving a stop measurement command, last_automeas_number would be equal to 31. Although the aspects using backwards calculation are described above as performing temperature and voltage correction, this is not required, and some alternative aspects using backwards calculation may (a) correct for changes to the frequency of the clock 830 due only to one of temperature and voltage changes or (b) perform neither temperature nor voltage correction (e.g., by assuming a stable clock frequency).

In some further alternative aspects, the apparatus may calculate the time stamps in step 2410 using a combination of forward and backward calculation. In some aspects, the apparatus (e.g., a computer of the apparatus) may use a combination of forward and backward calculation that calculates a time stamp for the nth set of sensor measurements using the following equation that averages the forward and backward calculated time stamps:

$$automeas\_time ( n ) = ( {automeas\_time}_{forward} ( n ) + {automeas\_time}_{backward} ( n ) ) / 2 \qquad ( 31 )$$

In some alternative aspects combining forward and backward calculation, the apparatus (e.g., a computer of the apparatus) may weight forward and backward calculation by recency (e.g., older measurement use more forward calculation, and recent measurements use more backward calculation). For example, the apparatus may calculate a time stamp for the nth set of sensor measurements using the following equations:

$$automeas\_time ( n ) = ( w 1 * {automeas\_time}_{forward} ( n ) + w 2 * {automeas\_time}_{backward} ( n ) ) \qquad ( 32 )$$

$$w 2 = {automeas\_time}_{backward} \text{} ( n ) / ( time\_stopped - time\_since \_last - time\_start ) \qquad ( 33 )$$

$$w 2 = 1 - w 1 \qquad ( 34 )$$

Although the aspects combining forward and backward calculation are shown above as not performing temperature and voltage correction (e.g., by assuming a stable clock frequency), this is not required, and some alternative aspects using combining forward and backward calculation may correct for changes to the frequency of the clock 830 due to (a) only one of temperature and voltage changes or (b) both of temperature and voltage changes. In some aspects combining forward and backward calculation, one or more coefficients may be calibrated so that forward and backward calculation align (e.g., via optimization through, for example and without limitation, least squares).

In some aspects, as shown in FIG. 24, the process 2400 may include a step 2412 in which the apparatus (e.g., the transceiver 101, the display device 105, and/or DMS 121) calculates analyte concentrations based on the received sets of sensor measurements and the calculated time stamps. In some aspects, a computer (e.g., the PIC controller 920 of the transceiver 101, the computer 310 of the display device 105, or a computer of the DMS 121) of the apparatus may calculate analyte concentrations based on the sets of sensor measurements received in step 2408 and the time stamps calculated in step 2410.

In some aspects, the sets of sensor measurements received in step 2408 may include measurements from a first sensing area and measurements from a second sensing area (e.g., one or more of the sets of sensor measurements may include measurements from the sensing areas 2202a and 2202c of the first sensing device 100A of the analyte sensor 100, and/or one or more other sets of sensor measurements may include measurements from the sensing areas 2202b and 2202d of the second sensing device 100B). In some aspects, the apparatus (e.g., a computer of the apparatus) may be configured to, in calculating the analyte concentrations based on the sets of sensor measurements and the calculated time stamps in step 2412, calculate individual analyte concentrations for the first sensing area, calculate individual analyte concentrations for the second sensing area, and calculate combined analyte concentrations based on at least the individual analyte concentrations for the first and second sensing areas.

In some aspects (e.g., some aspects in which the analyte senor 100 includes only one sensing device), the sets of sensor measurements received in step 2408 may include only sets of sensor measurements conveyed by one sensing device of the analyte sensor 100. In some aspects in which the analyte sensor 100 includes multiple sensing devices, the sets of sensor measurements received in step 2408 may include sets of sensor measurements conveyed by multiple sensing devices. For example, in some aspects in which the analyte sensor 100 includes first and second sensing devices 100A and 100B, the sets of sensor measurements received in step 2408 may include sets of sensor measurements conveyed by the first sensing device 100A of the analyte sensor 100 and sets of sensor measurements conveyed by the second sensing device 100B of the analyte sensor 100. In some aspects, the apparatus (e.g., a computer of the apparatus) may be configured to calculate the analyte concentrations in step 2412 based on the sets of sensor measurements conveyed by the first sensing device 100A, the sets of sensor measurements conveyed by the second sensing device 100B, and the time stamps calculated in step 2410.

In some aspects in which the apparatus receives sets of sensor measurements from the first and second sensing devices 100A and 100B, the sets of sensor measurements conveyed by the first sensing device 100A and received by the apparatus in step 2408 may include measurements from a first sensing area 2202a of the first sensing device 100A and measurements from a second sensing area 2202c of the first sensing device 100A. In some aspects, the sets of sensor measurements conveyed by the second sensing device 100B and received by the apparatus in step 2408 may include measurements from a first sensing area 2202b of the second sensing device 100B and measurements from a second sensing area 2202d of the second sensing device 100B. In some aspects, the apparatus (e.g., a computer of the apparatus) may be configured to, in calculating the analyte concentrations based on the sets of sensor measurements and the calculated time stamps in step 2412: (1) calculate individual analyte concentrations for the first sensing area 2202a of the first sensing device 100A; (2) calculate individual analyte concentrations for the second sensing area 2202c of the first sensing device 100A; (3) calculate individual analyte concentrations for the first sensing area 2202b of the second sensing device 100B; (4) calculate individual analyte concentrations for the second sensing area 2202d of the second sensing device 100B; and (5) calculate combined analyte concentrations based on the individual analyte concentrations for the first and second sensing areas 2202a and 2202c of the first sensing device 100A and the individual analyte concentrations for the first and second sensing areas 2202b and 2202d of the second sensing device 100B. In some aspects, a combined analyte concentration may calculated based on a weighted average of the individual analyte concentrations for the sensing areas 2202a-2202d. In some aspects, the apparatus (e.g., a computer of the apparatus) may use sensing area-specific health metrics that assess noise, foreign body response (FBR) degradation (e.g., as measured using interferent indicators 209), and/or stability of reference channels. In some aspects, the apparatus (e.g., a computer of the apparatus) may determine the quality of each of the sensing areas 2202a, 2202b, 2202c, and 2202d and selectively de-weight underperforming areas (e.g., sensing area 2202d) when calculating the combined analyte concentration.

In some aspects, calculating each of the analyte concentrations in step 2412 may include (i) calculating interstitial fluid analyte concentrations based on the sets of sensor measurements received in step 2408, (ii) calculating interstitial fluid analyte concentration rates-of-change based on the interstitial analyte concentrations and the time stamps calculated in step 2410, and (iii) calculating blood analyte concentrations based on the calculated interstitial analyte concentrations and the calculated interstitial fluid analyte concentration rates-of-change. In some aspects, calculating the interstitial fluid analyte concentration rate-of-change for the most-recent interstitial fluid analyte concentration may be based on the most-recent interstitial fluid analyte concentration and one or more less-recent interstitial fluid analyte concentrations (e.g., using a causal method with “backward difference” derivative calculation). In some aspects, calculating the interstitial fluid analyte concentration rate-of-change for an interstitial fluid analyte concentration other than the most-recent interstitial fluid analyte concentration may be based on the historical interstitial fluid analyte concentration, one or more less recent interstitial fluid analyte concentrations, and one or more recent interstitial fluid analyte concentrations (e.g., using an acausal centered difference for derivative calculations).

Aspects of the present invention have been fully described above with reference to the drawing figures. Although the invention has been described based upon these preferred aspects, it would be apparent to those of skill in the art that certain modifications, variations, and alternative constructions could be made to the described aspects within the spirit and scope of the invention. For example, although the aspects of the invention in which the analyte indicator 207 and interferent indicator 209 are distributed throughout the same indicator element 106, this is not required. In some alternative aspects, the sensing devices of the analyte sensor 100 may include a first indicator element that includes the analyte indicator 207 and a second indicator element that includes the interferent indicator 209. In these alternative aspects, the analyte indicator 207 and the interferent indicator 209 may be spatially separated from one another.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel. For example, although the step 2404 of conveying one or more start measurement commands is shown in FIG. 24 as being performed after steps 2410 and 2412, this is not required, and, in some alternative aspects, step 2404 may be performed before or in parallel with step 2410 and/or step 2412.

What is claimed is:

1. An apparatus comprising:
an interface device;
wherein the apparatus is configured to:
use the interface device to receive sets of sensor measurements conveyed by an analyte sensor, wherein the analyte sensor takes sets of sensor measurements at a first frequency that has a period equal to a threshold number of cycles of a clock of the analyte sensor;
calculate time stamps for the sets of sensor measurements; and
calculate analyte concentrations based on the sets of sensor measurements and the calculated time stamps.

2. The apparatus of claim 1, wherein each of the sets of sensor measurements includes timing information, and the apparatus is configured to use at least the timing information of the sets of sensor measurements to calculate the time stamps for the sets of sensor measurements.

3. The apparatus of claim 2, wherein the timing information of a set of sensor measurements includes a count of the cycles of the clock at the time the set of sensor measurements was taken.

4. The apparatus of claim 2, wherein the timing information of a set of sensor measurements includes a number n for the set of sensor measurements.

5. The apparatus of 2, wherein each of the sets of sensor measurements comprises a temperature measurement, and the apparatus is configured to use at least the timing information and one or more of the temperature measurements of the sets of sensor measurements to calculate the time stamps for the sets of sensor measurements.

6. The apparatus of claim 5, wherein the apparatus is configured to use at least the timing information and the one or more of the temperature measurements of the sets of sensor measurements and a characterization of a temperature dependence of the cycles of the clock of the analyte sensor to calculate the time stamps for the sets of sensor measurements.

7. The apparatus of claim 6, wherein the apparatus is configured to use at least the timing information and the one or more of the temperature measurements of the sets of sensor measurements, the characterization of the temperature dependence of the cycles of the clock of the analyte sensor, and one or both of a time at which the apparatus conveyed a start sensor measurement command to the analyte sensor and a time at which the apparatus conveyed a stop measurement command to the analyte sensor to calculate the time stamps for the sets of sensor measurements.

8. The apparatus of claim 6, wherein the apparatus is configured to use at least the timing information and the one or more of the temperature measurements of the sets of sensor measurements, the characterization of the temperature dependence of the cycles of the clock of the analyte sensor, and the first frequency to calculate the time stamps for the sets of sensor measurements.

9. The apparatus of 2, wherein each of the sets of sensor measurements comprises a voltage measurement, the voltage measurement is a measurement of a voltage produced by a charge storage device of the analyte sensor, and the apparatus is configured to use at least the timing information and one or more of the voltage measurements of the sets of sensor measurements to calculate the time stamps for the sets of sensor measurements.

10. The apparatus of claim 9, wherein the apparatus is configured to use at least the timing information and the one or more of the voltage measurements of the sets of sensor measurements and a characterization of a voltage dependence of the cycles of the clock of the analyte sensor to calculate the time stamps for the sets of sensor measurements.

11. The apparatus of claim 10, wherein the apparatus is configured to use at least the timing information and the one or more of the voltage measurements of the sets of sensor measurements, the characterization of the voltage dependence of the cycles of the clock of the analyte sensor, and one or both of a time at which the apparatus conveyed a start sensor measurement command to the analyte sensor and a time at which the apparatus conveyed a stop measurement command to the analyte sensor to calculate the time stamps for the sets of sensor measurements.

12. The apparatus of claim 10, wherein the apparatus is configured to use at least the timing information and the one or more of the voltage measurements of the sets of sensor measurements, the characterization of the voltage dependence of the cycles of the clock of the analyte sensor, and the first frequency to calculate the time stamps for the sets of sensor measurements.

13. The apparatus of claim 1, wherein:
the sets of sensor measurements include first sets of sensor measurements, which were stored by the analyte sensor at the first frequency, and second sets of sensor measurements, which were stored by the analyte sensor at a second frequency that is less than the first frequency; and
the apparatus is configured to, in calculating the time stamps for the sets of sensors measurements, calculate time stamps for the first sets of sensor measurements and calculate time stamps for the second sets of sensor measurements.

14. The apparatus of claim 13, wherein the first sets of sensor measurements are more recent sets of sensor measurements than the second sets of sensor measurements.

15. The apparatus of claim 1, wherein the apparatus is further configured to use the interface device to convey one or more measurement read requests, and the sets of sensor measurements are received in response to the one or more measurement read requests.

16. The apparatus of claim 15, wherein the apparatus is further configured to:
use the interface device to convey one or more stop sensor measurement commands before conveying the one or more measurement read requests; and
use the interface device to convey one or more start sensor measurement commands after conveying the one or more measurement read requests and receiving the sets of sensor measurements.

17. The apparatus of claim 1, wherein:
the sets of sensor measurements include measurements from a first sensing area and measurements from a second sensing area, and
the apparatus is configured to, in calculating the analyte concentrations based on the sets of sensor measurements and the calculated time stamps, calculate individual analyte concentrations for the first sensing area, calculate individual analyte concentrations for the second sensing area, and calculate combined analyte concentrations based on at least the individual analyte concentrations for the first and second sensing areas.

18. The apparatus of claim 1, wherein:
the sets of sensor measurements include sets of sensor measurements conveyed by a first sensing device of the analyte sensor and sets of sensor measurements conveyed by a second sensing device of the analyte sensor; and
the apparatus is configured to calculate the analyte concentrations based on the sets of sensor measurements conveyed by the first sensing device of the analyte sensor, the sets of sensor measurements conveyed by the second sensing device of the analyte sensor, and the calculated time stamps.

19. The apparatus of claim 18, wherein:
the sets of sensor measurements conveyed by the first sensing device include measurements from a first sensing area of the first sensing device and measurements from a second sensing area of the first sensing device;
the sets of sensor measurements conveyed by the second sensing device include measurements from a first sensing area of the second sensing device and measurements from a second sensing area of the second sensing device; and
the apparatus is configured to, in calculating the analyte concentrations:
calculate individual analyte concentrations for the first sensing area of the first sensing device;
calculate individual analyte concentrations for the second sensing area of the first sensing device;
calculate individual analyte concentrations for the first sensing area of the second sensing device;
calculate individual analyte concentrations for the second sensing area of the second sensing device; and
calculate combined analyte concentrations based on at least the individual analyte concentrations for the first and second sensing areas of the first sensing device and the individual analyte concentrations for the first and second sensing areas of the second sensing device.

20. The apparatus of claim 1, further comprising a computer including a non-transitory memory and processing circuitry.

21. A system comprising:
the apparatus of claim 1; and
the analyte sensor.

22. A method comprising:
using an interface device of an apparatus to receive sets of sensor measurements conveyed by an analyte sensor, wherein the analyte sensor takes sets of sensor measurements at a first frequency that has a period equal to a threshold number of cycles of a clock of the analyte sensor;
using the apparatus to calculate time stamps for the sets of sensor measurements; and
using the apparatus to calculate analyte concentrations based on the sets of sensor measurements and the calculated time stamps.
